(19) United States
(12) Patent Application Publication
(10) Pub. No.: US 2007/0020638 A1
(43) Pub. Date: Jan. 25, 2007

(54) Variants of the ADAM 12 gene

(76) Inventors: Mike Owen, Cardiff (GB); Julie Williams, Cardiff (GB); Michael O'Donovan, Cardiff (GB); Denise H. Harold, Cardiff (GB); Simon Lovestone, London (GB)

Correspondence Address: KING & SCHICKLI, PLLC, 247 NORTH BROADWAY, LEXINGTON, KY, 40507, US

(21) Appl. No.: 11/083,416

(22) Filed: Mar. 18, 2005

(30) Foreign Application Priority Data
Mar. 19, 2004 (GB) GB 0406180.0
May 14, 2004 (GB) GB 0410761.1

(51) Int. Cl. C12Q 1/68

(52) U.S. Cl. 435/6

(57) ABSTRACT

The invention relates to variants of the ADAM12 gene and the role these variants have to play in the diagnosis and treatment of a number of conditions including cancer, disorders of myogenesis, adipogenesis, cardiac hypertrophy and Alzheimer's disease, and in particular, Late Onset Alzheimer's Disease.

The invention relates to variants of the ADAM 12 gene and their corresponding proteins. Further, the invention relates to the use of information concerning these variants in the diagnosis of diseases associated therewith, particularly, but not exclusively, Alzheimer's disease (AD). Additionally, the invention relates to research tools for identifying therapies to treat the aforementioned diseases, which rely on a knowledge of the aforementioned variants, and therapies derived therefrom or relating thereto. The invention further includes means for detecting or assessing any of the aforementioned variants and executing any of the aforementioned diagnoses or therapies.

The ADAM (A Disintegrin And Metalloprotease) family of proteins are highly conserved, multi-functional, type I transmembrane and secreted proteins that typically contain a N-terminal secretion signal, and pro-, metalloprotease, disintegrin-like, cysteine-rich, epidermal growth factor like, transmembrane and cytoplasmic domains. ADAM proteins are converted from a latent proform into an active enzyme, prior to secretion, as a result of prodomain cleavage in the trans-Golgi apparatus of the cell. At least six members of the ADAM family of proteins have been shown to have proteolytic activity including ADAM 12. ADAM17 releases soluble tumour necrosis factor alpha from its membrane precursor, ADAM10 cleaves Delta, a ligand of the Notch receptor, and ADAM19 cleaves membrane-anchored neuregulin. ADAM9, ADAM10 and ADAM17 also participate in the alpha-secretase cleavage of amyloid precursor protein.

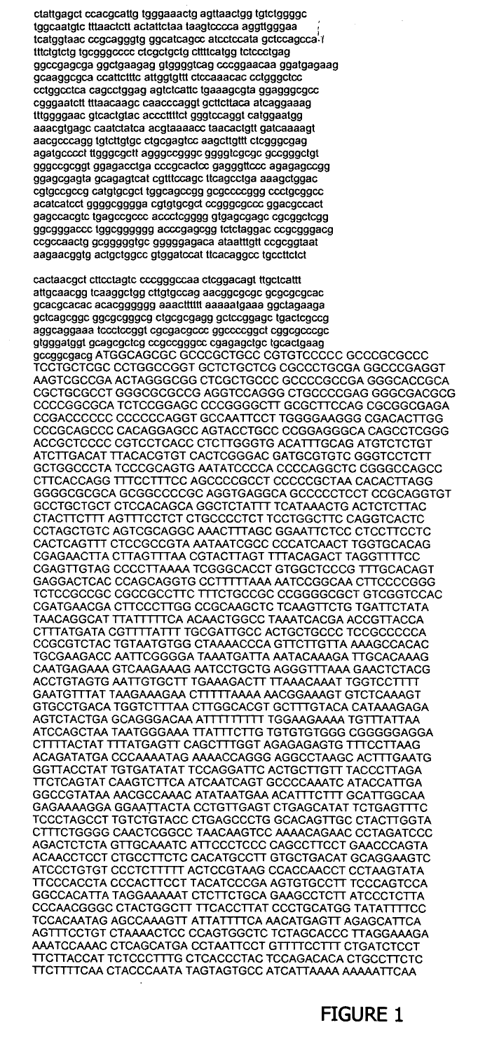

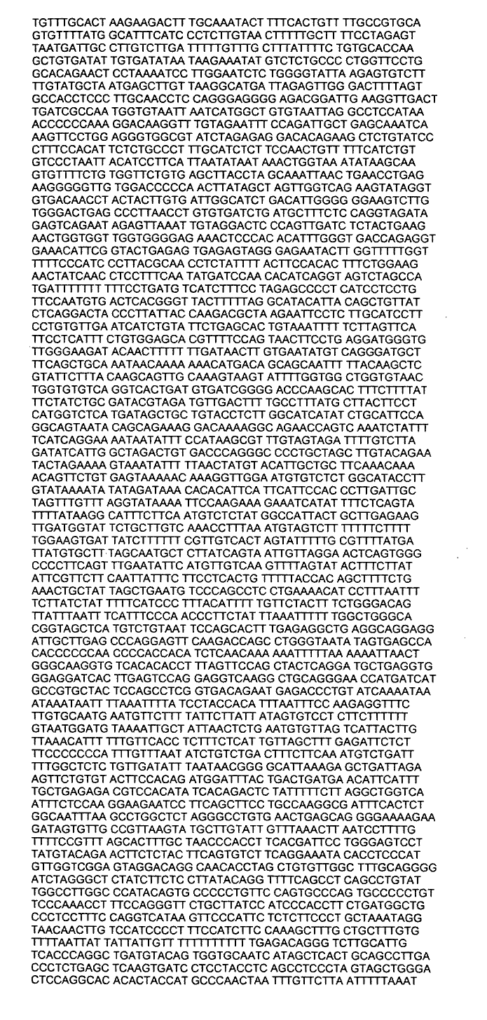

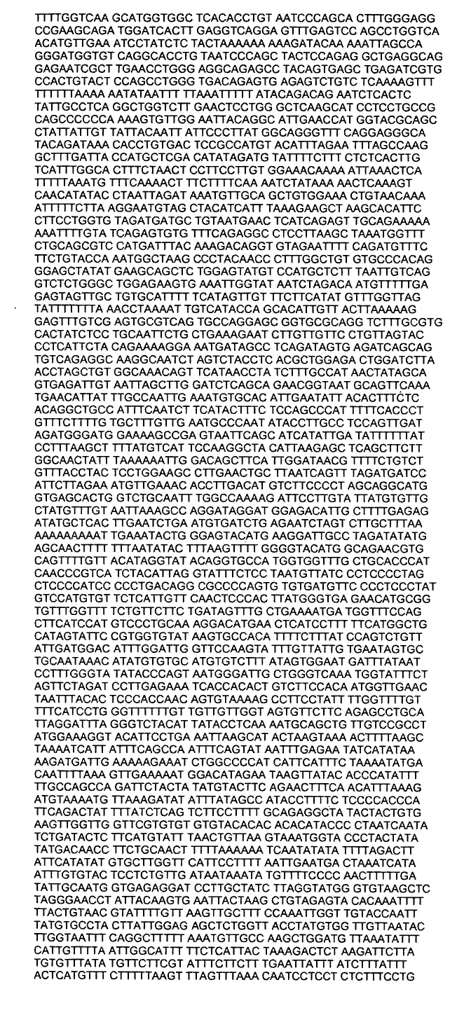

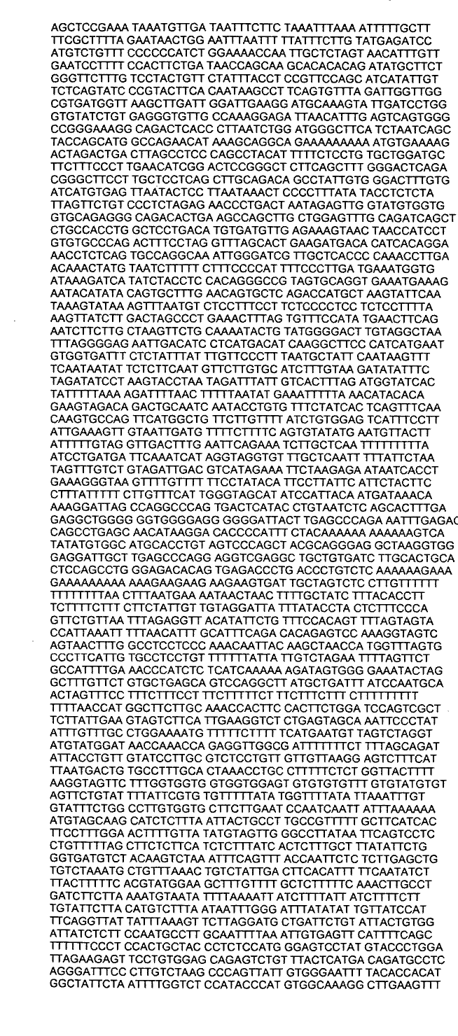

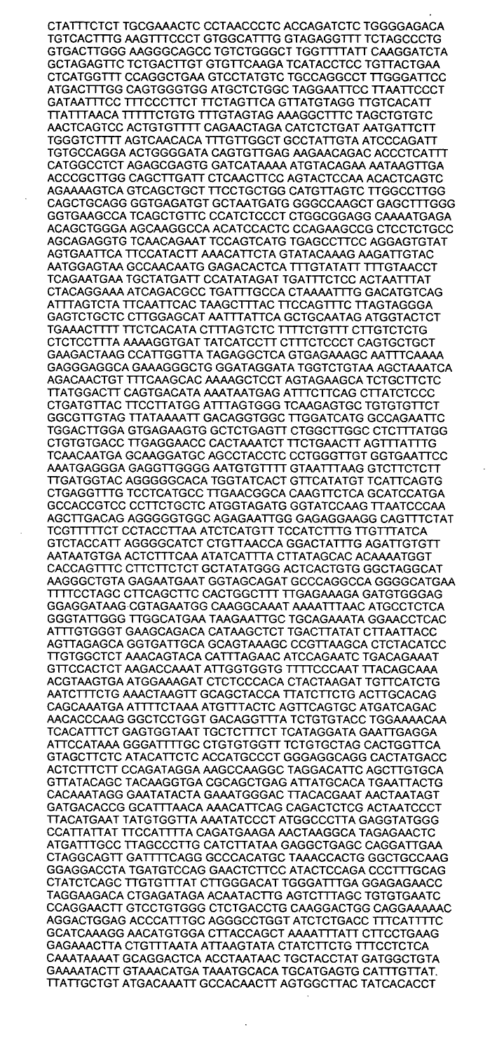

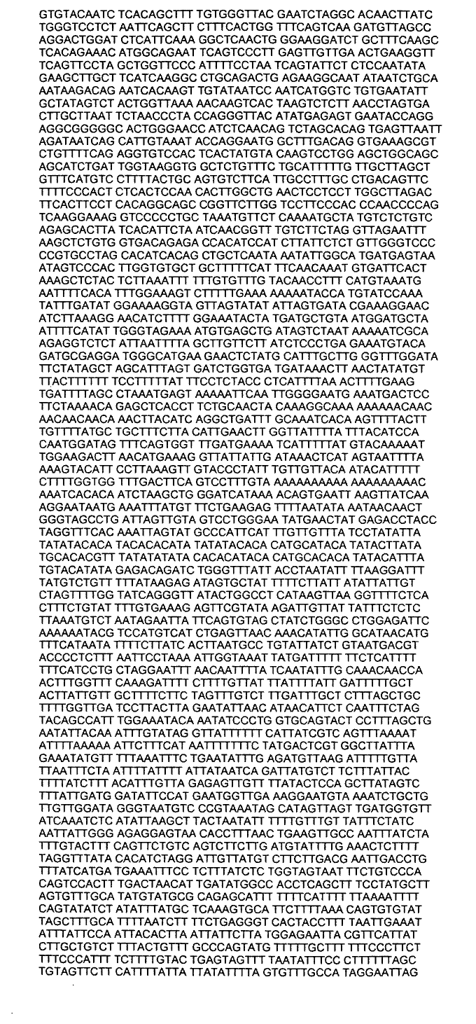

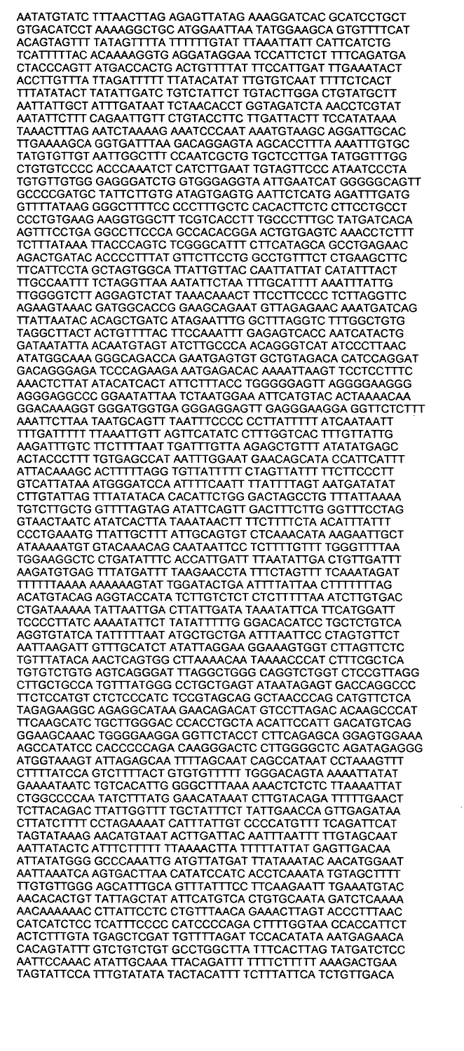

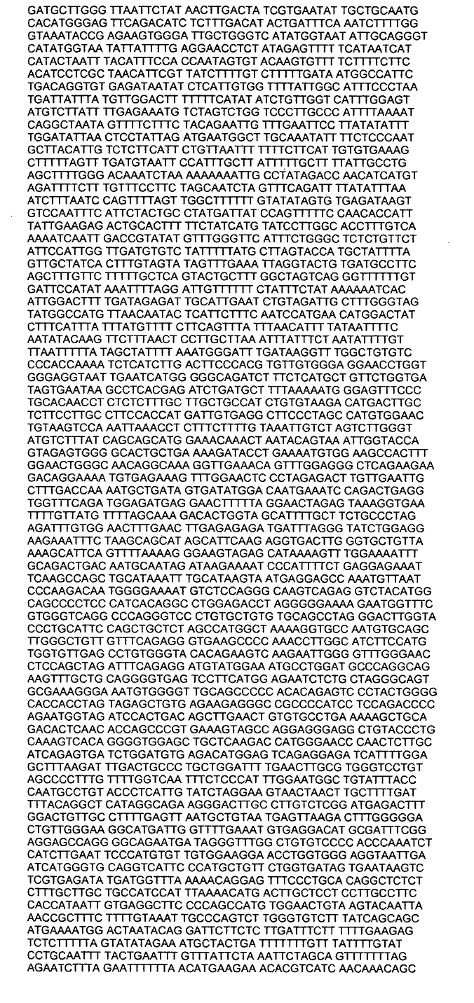

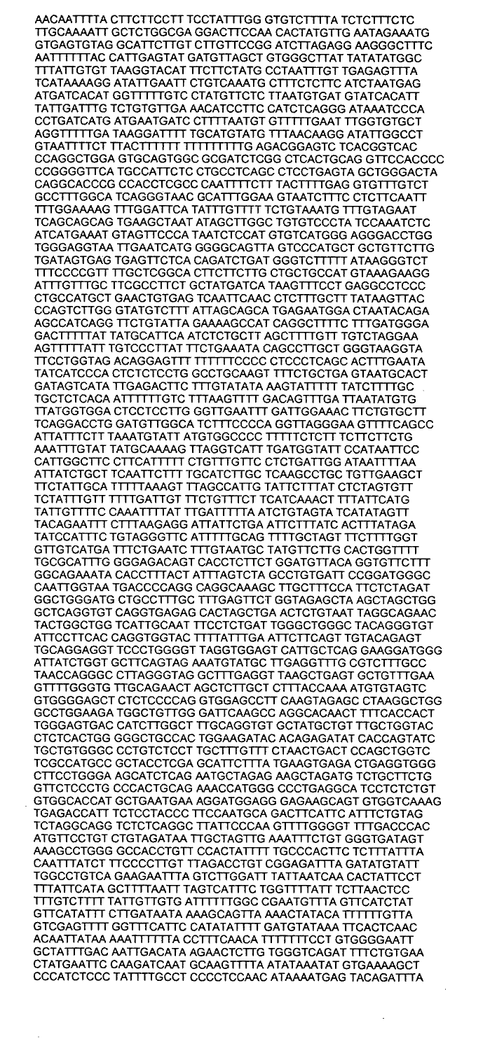

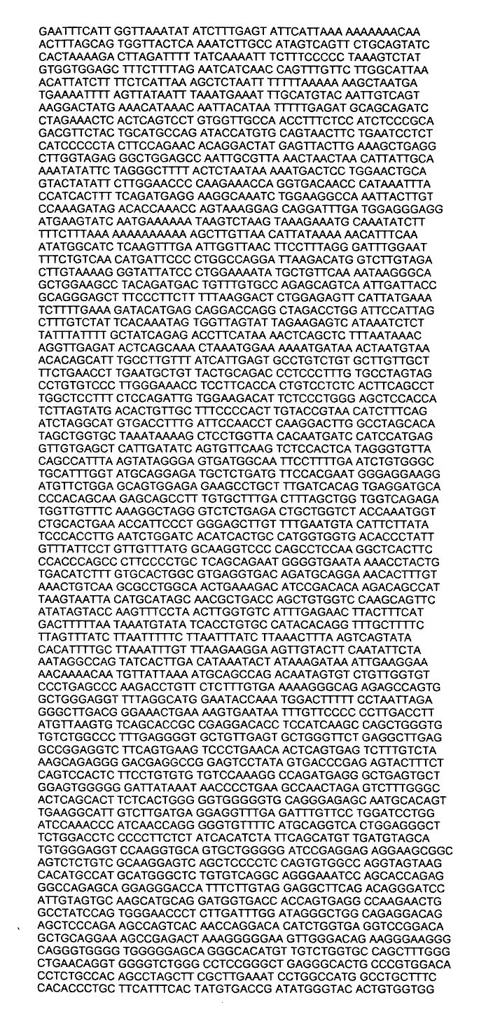

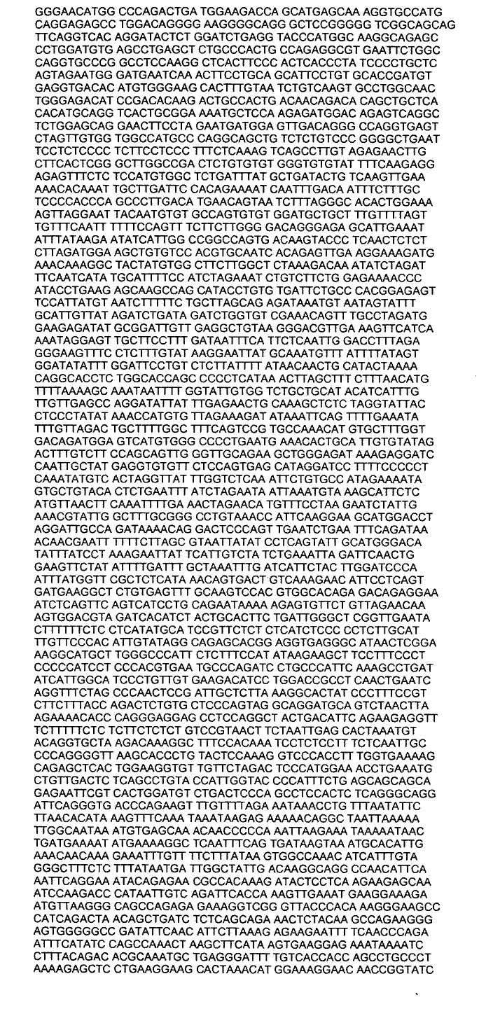

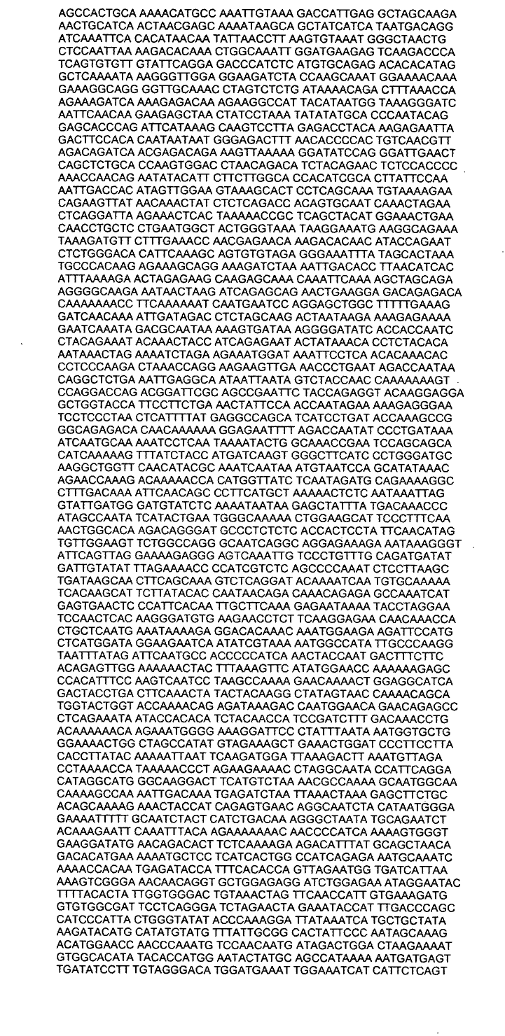

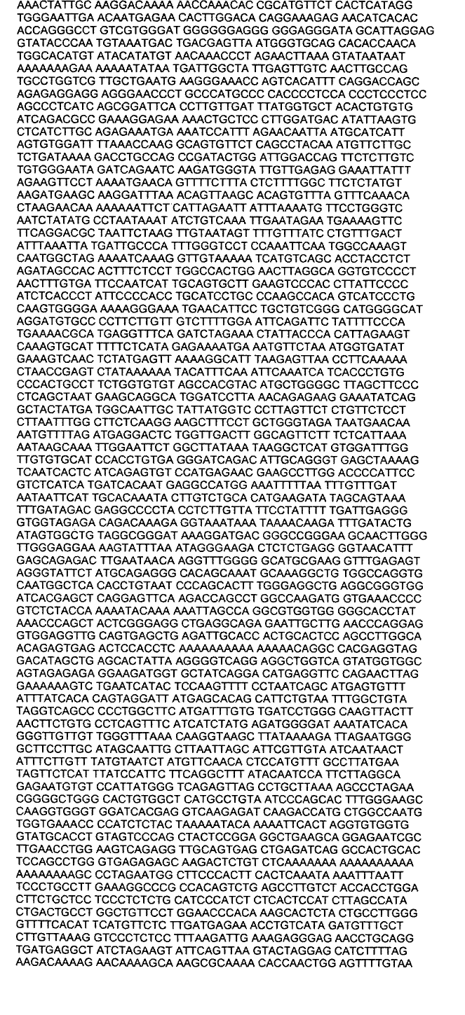

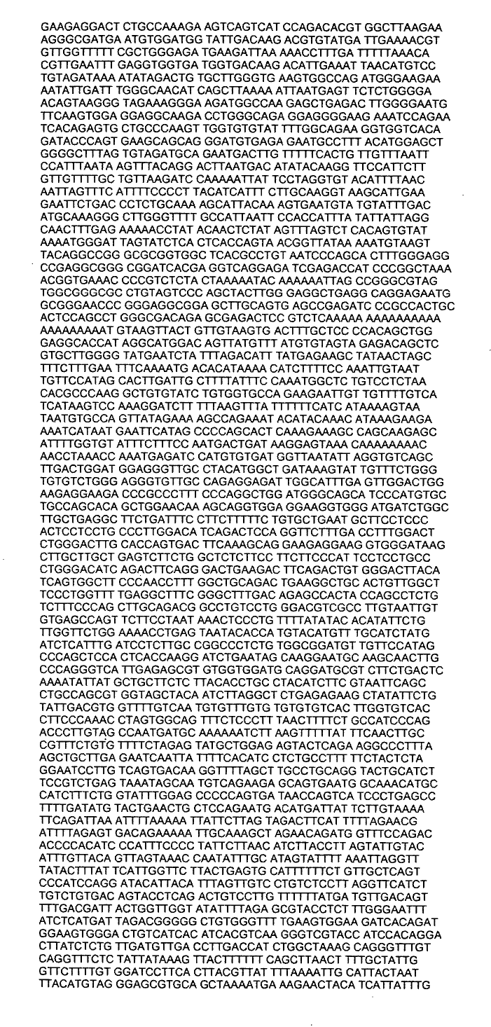

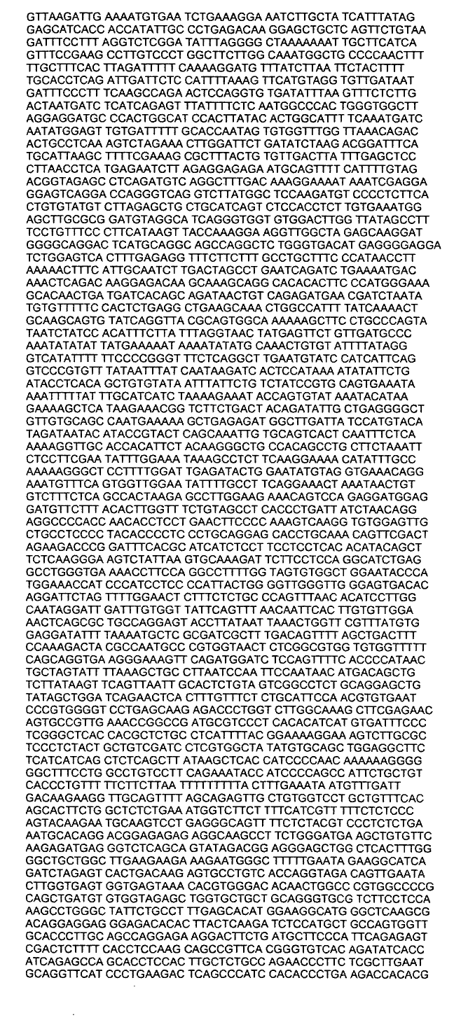

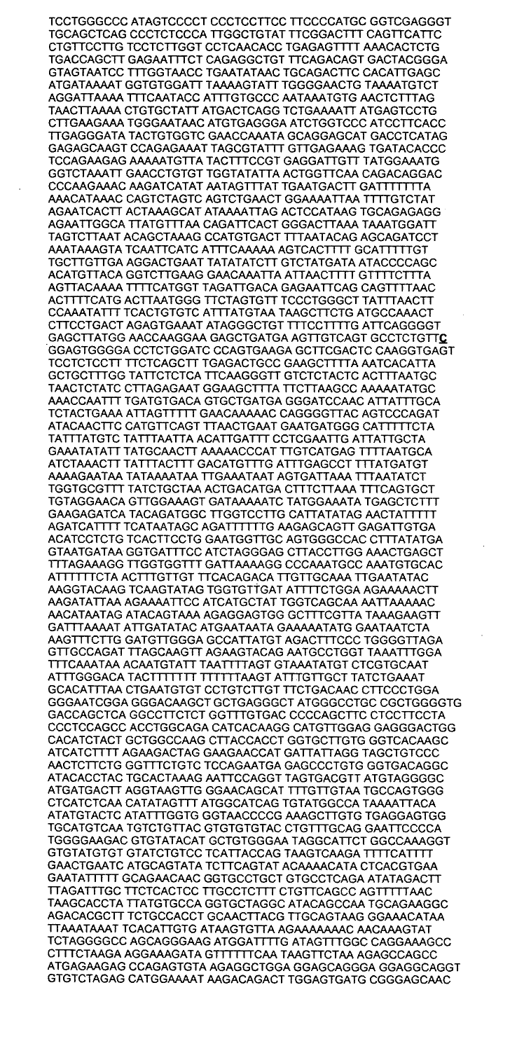

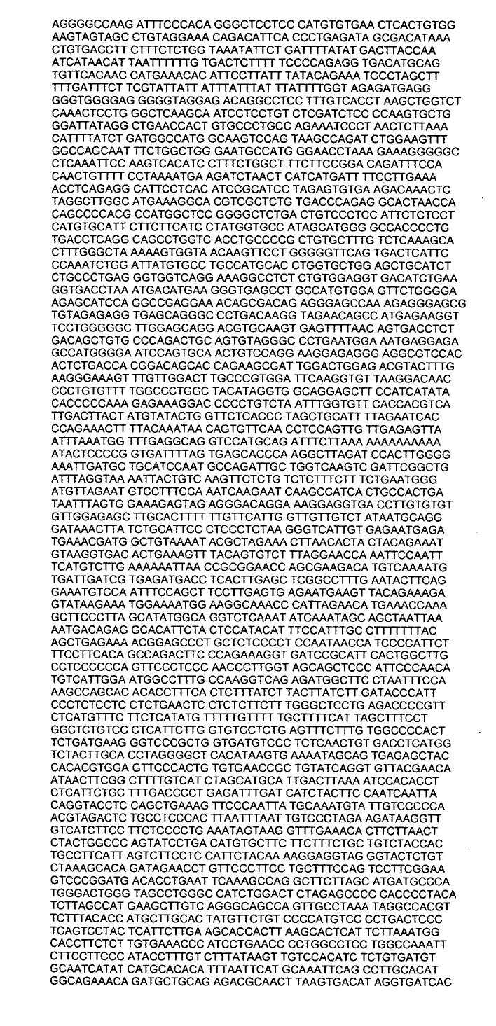

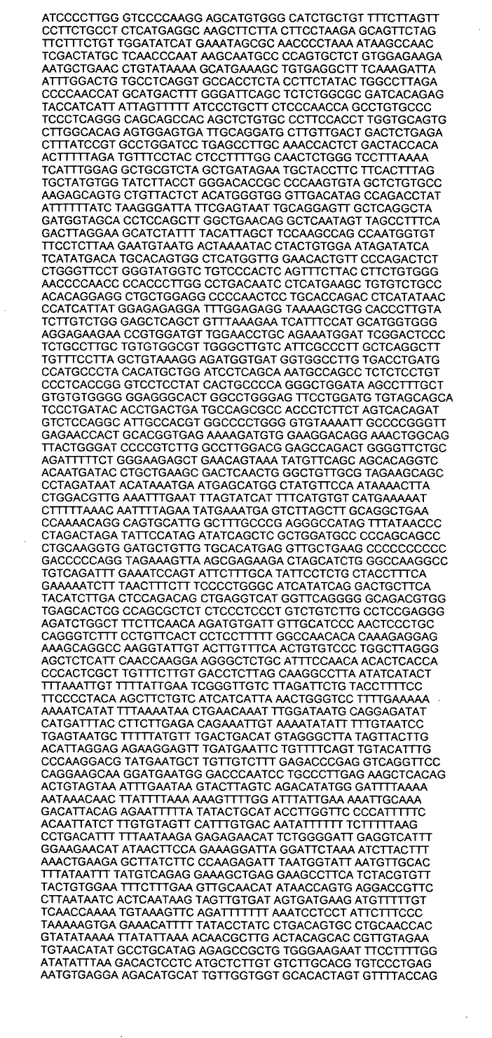

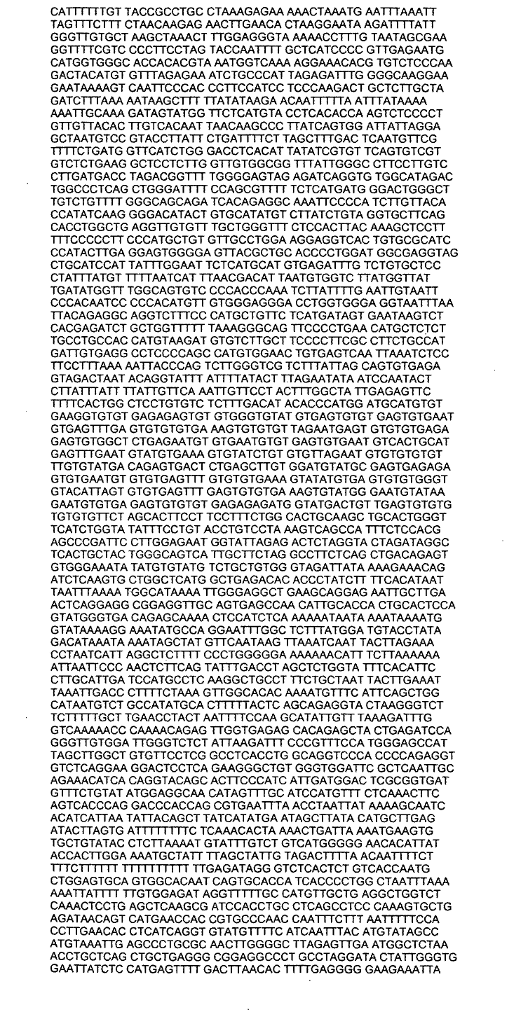

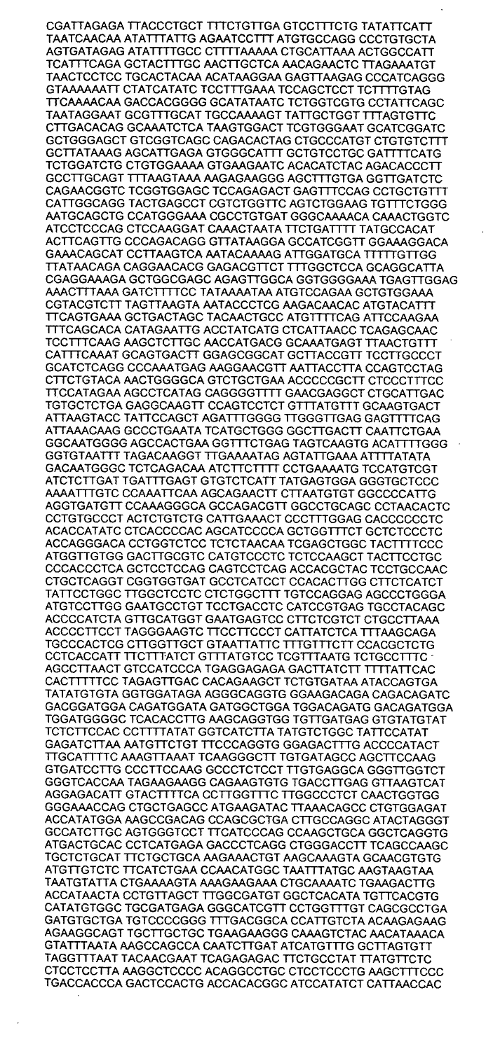

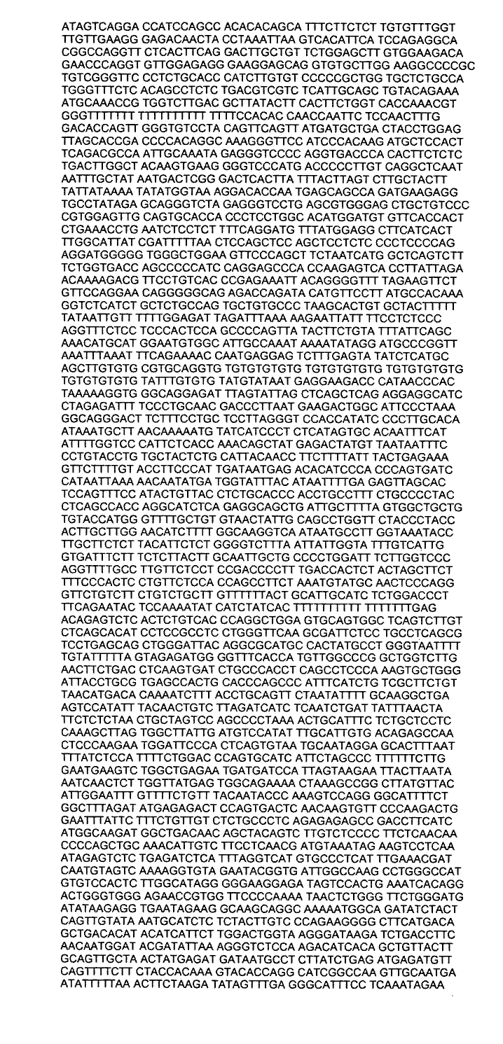

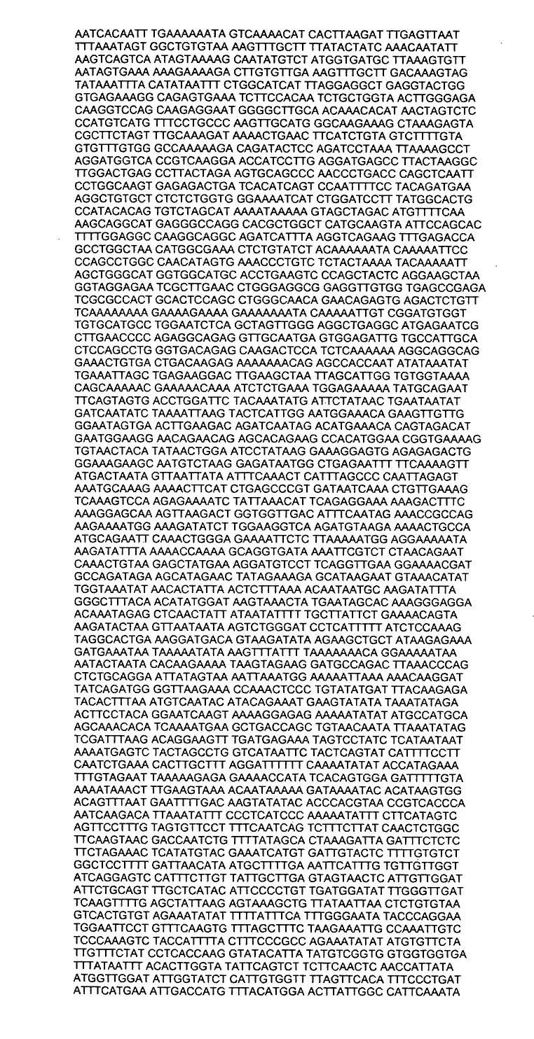

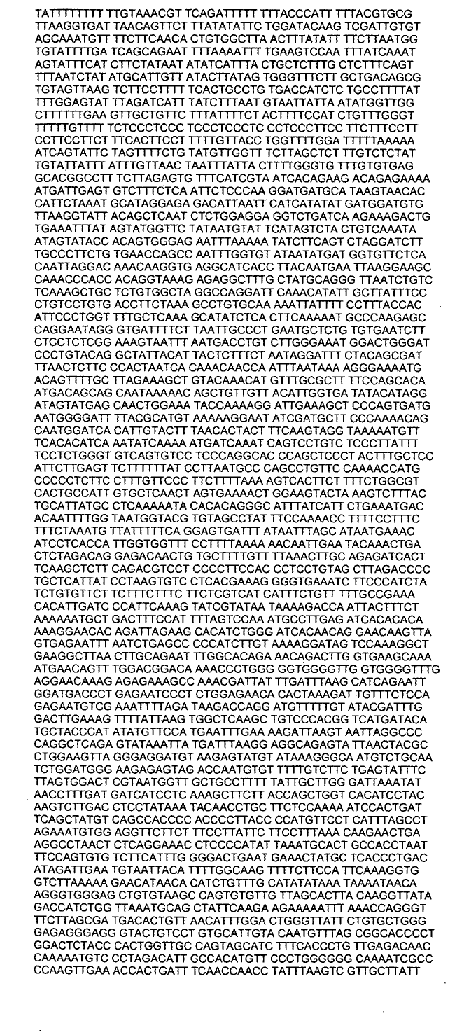

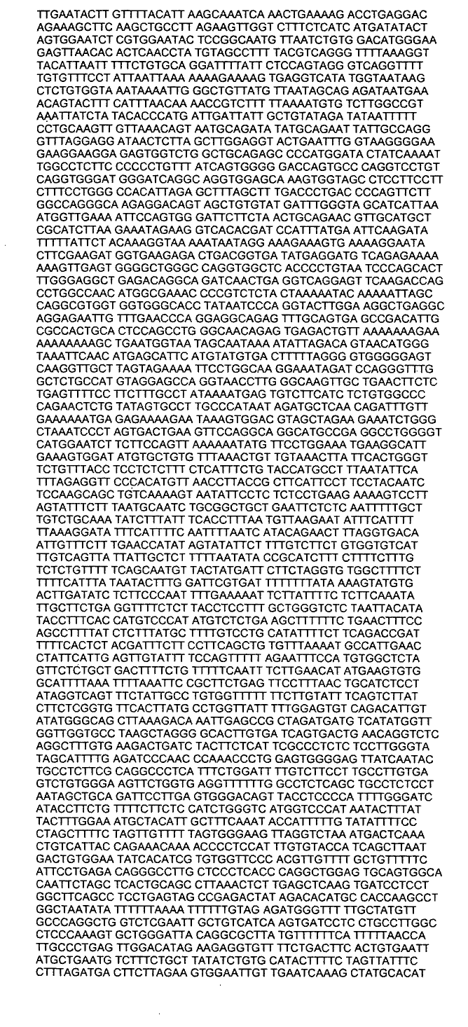

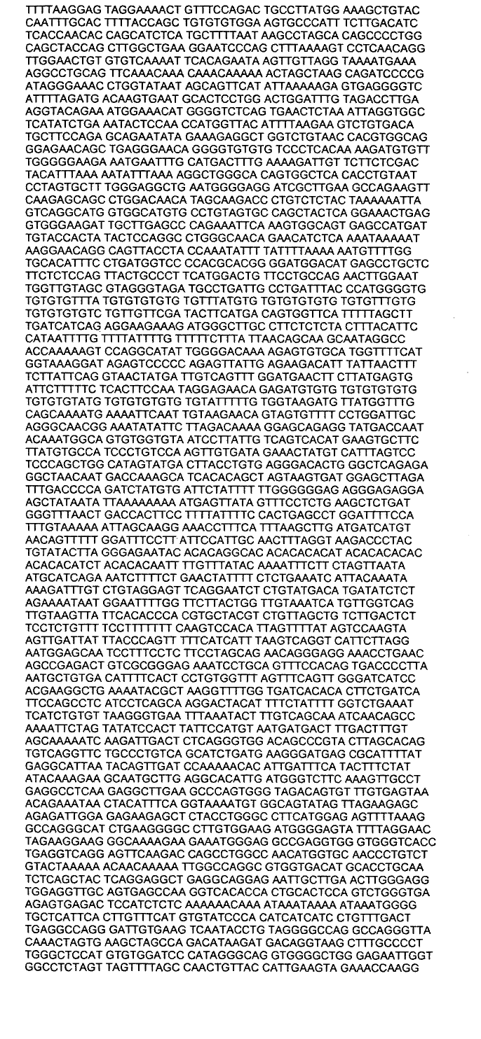

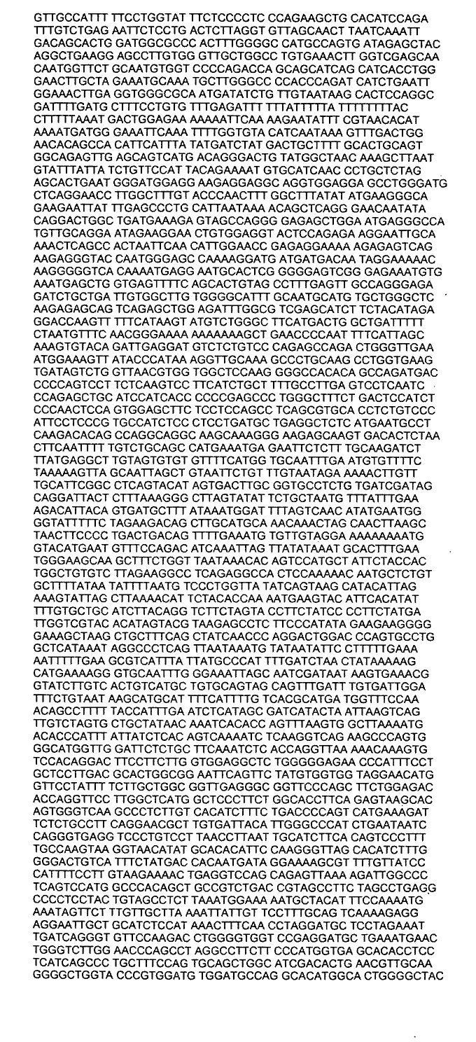

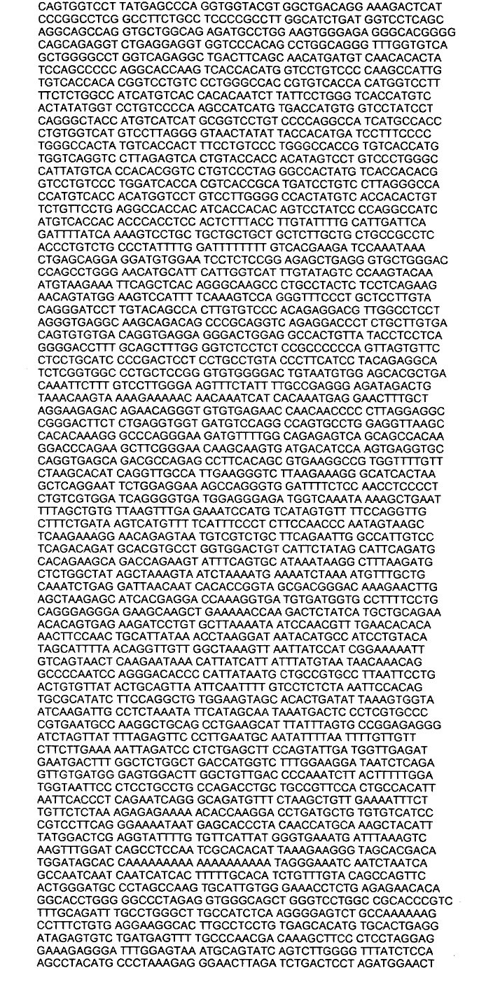

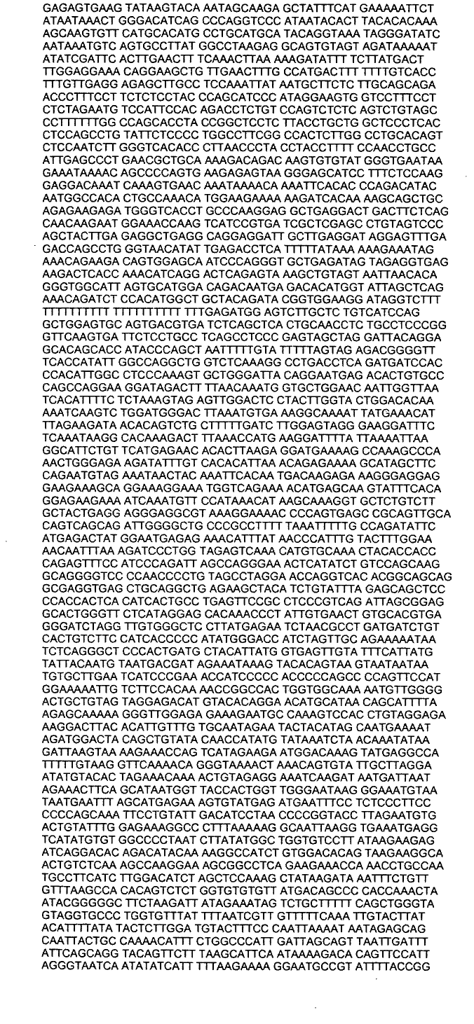

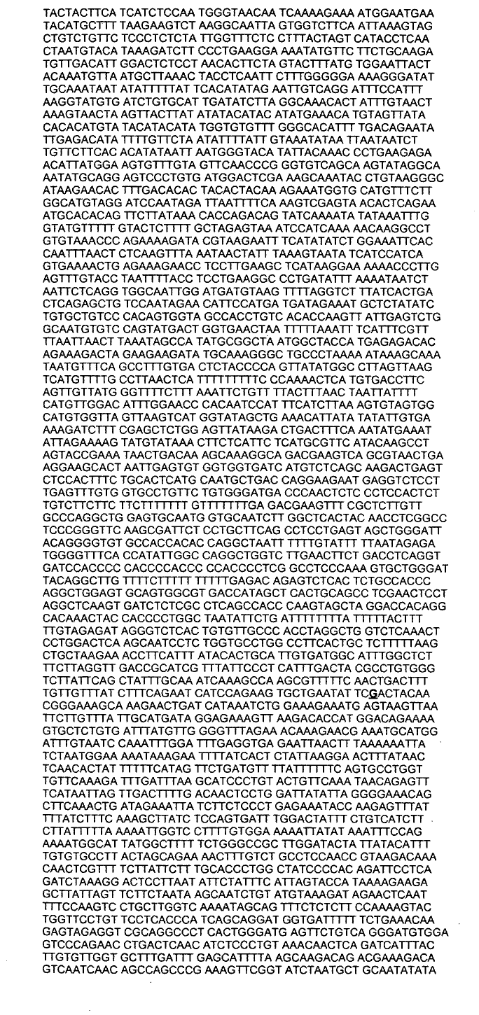

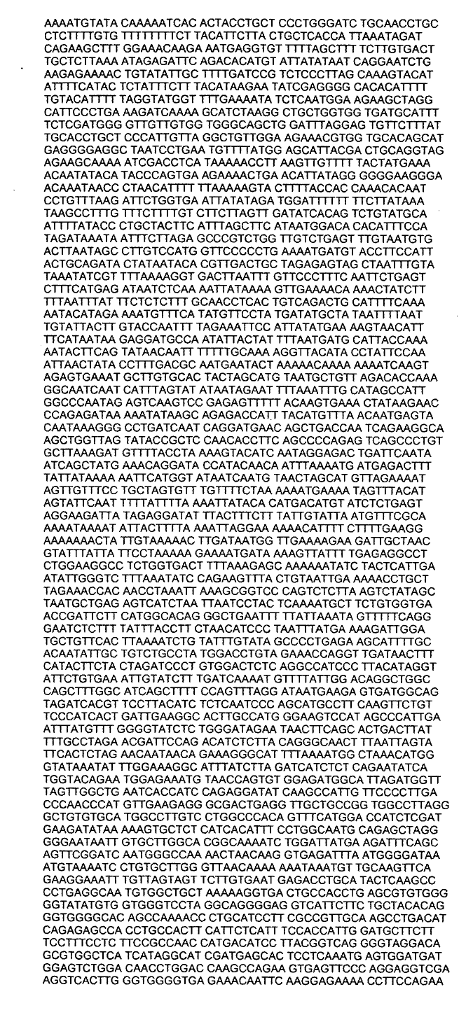

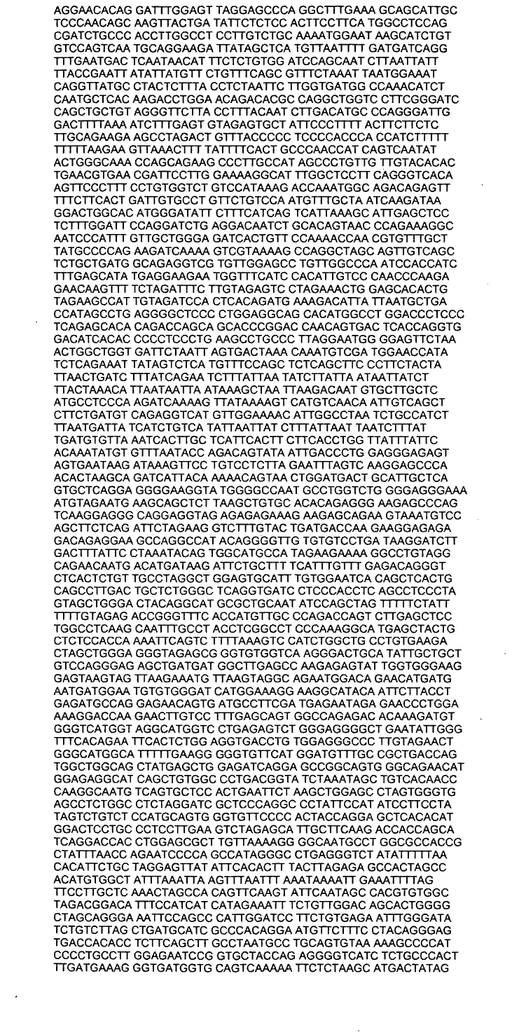

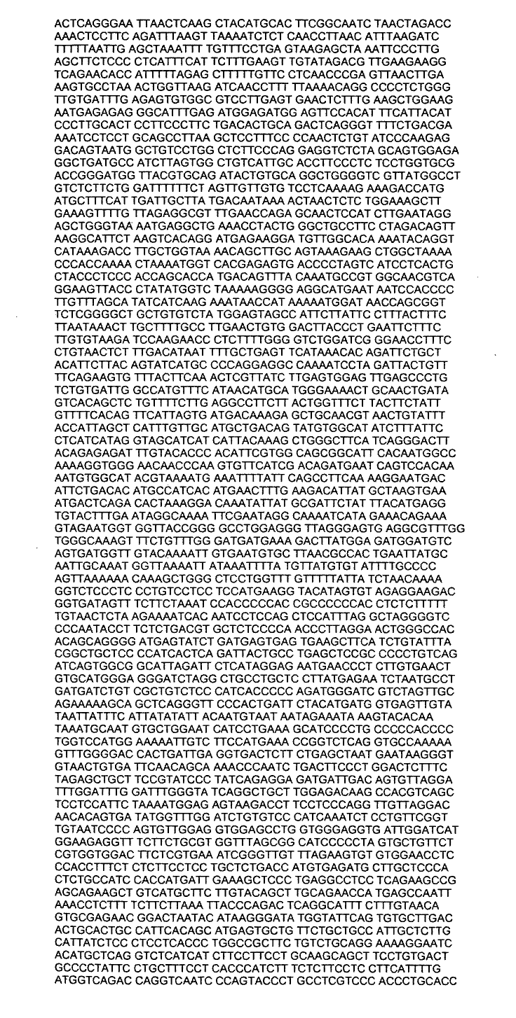

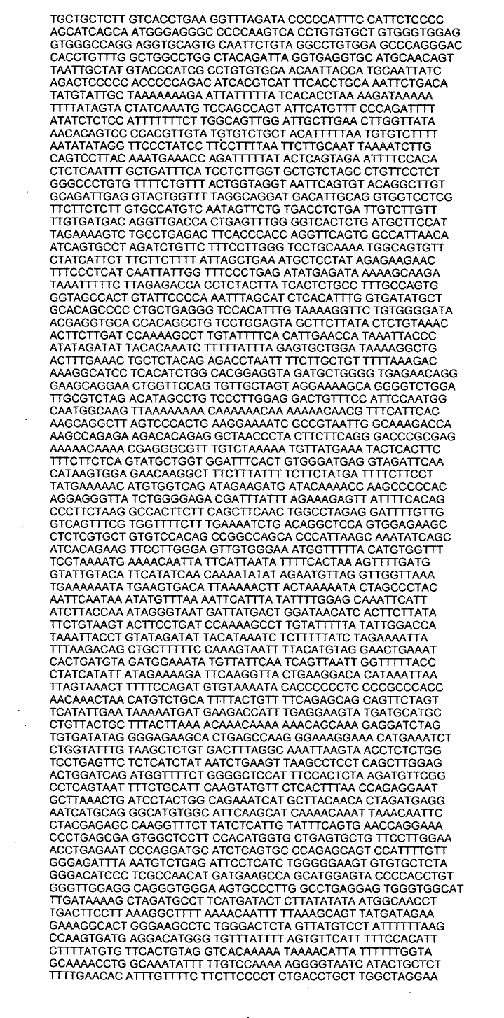

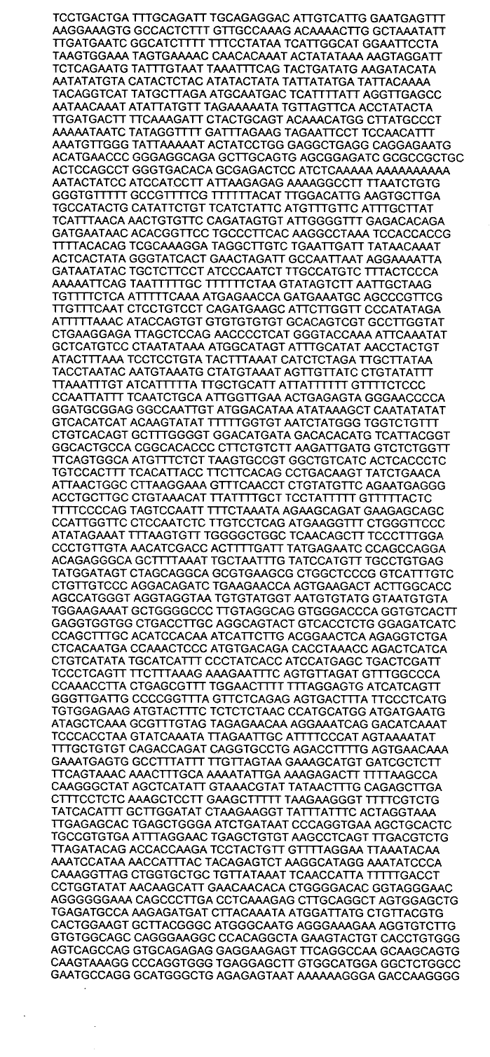

The DNA sequence structure of ADAM 12 is shown in FIG. 1.

ADAM 12 is known to cleave insulin-like growth factor binding proteins IGFBP-3 and IGFBP-5 as well as the heparin-binding epidermal growth factor (HB-EGF). Recently, it has been demonstrated that inhibitors of the ADAM12 processing of HB-EGF attenuate cardiac hypertrophy (Asakura et al., Nat Med. 2002 January; 8(1):35-40). The ADAM 12 gene encodes two known splice variants: a membrane-bound form designated ADAM12-L, and a shorter form designated ADAM12-S, which is secreted as a soluble protein. ADAM12 has been implicated in the differentiation of mesenchymal cells such as skeletal myoblasts and osteoblasts (Gilpin B J et al., J Biol Chem 1998 January 2;273(1):157-66; Inoue et al., J Biol Chem 1998 273:4180-4187). ADAM12 expression is strongly upregulated in human carcinomas, suggesting that ADAM 12 plays a pivotal role in cell-cell, and cell-matrix interactions (Iba K et al., Am J Pathol 1999 154:1489-1501). In addition, ADAM12 is abundantly expressed in human placenta and is present in the maternal circulation (Zengdun S et al., J Biol Chem 2000 24:18574-18580). Phenotypic analysis of mice deficient for ADAM12 (created by gene-targeting), suggest that the gene may be implicated in regulating adipogenesis and myogenesis through a linked developmental pathway (Kurisaki et al., Mol Cell Biol 2003 23:55-61).

The human version of ADAM 12 shares high homology with its mouse orthologue (81% amino acid identity) and with other members of the ADAM family (example 45% amino acid identity with ADAM 9). Similar to other ADAM proteases, ADAM 12 is synthesised as a latent zinc-dependent metalloprotease. This latency mechanism is achieved by means of a ‘cysteine switch’, whereby an unpaired cysteine residue in the prodomain directly coordinates the zinc ion at the catalytic site. Activation is initiated in the trans-Golgi network (TGN), when a furin-type endopeptidase cleaves the prodomain from the protease. The ADAM 12 prodomain is required for the export of the protein from the endoplasmic reticulum to the Golgi apparatus, and may be required for the correct folding of newly synthesised ADAM 12 into an active protease.

Numerous genetic linkage and association studies strongly support the existence of an Alzheimer's disease gene locus on chromosome 10. The ADAM12 gene maps to chromosome 10q and is therefore a positional candidate gene for Alzheimer's disease.

However, given the functional studies described above that identify a role for ADAM12 in cardiac hypertrophy, the differentiation of mesenchymal cells, its upregulation in human carcinomas, its abundant expression in human placenta and its involvement in adipogenesis and myogenesis there appears to be conflicting information in the public domain concerning the involvement that the ADAM12 gene may have in Alzheimer's Disease.

Despite this fact, we have focused on the role of ADAM 12 in Alzheimer's Disease, a progressive neurodegenerative disorder which accounts for more than half of all cases of dementia among people over 65 years of age.

Molecular genetic analyses have led to the discovery of three genes involved in early onset autosomal dominant AD: APP on chromosome 21, PSEN1 on chromosome 14 and PSEN2 on chromosome 1. The majority of AD cases, however, have an age at onset over 65 years and exhibit no clear, mendelian pattern of inheritance. The only widely accepted genetic risk factor for late onset AD (LOAD) is the ε4 allele of the apolipoprotein E (APOE) gene on chromosome 19. However, variation of the ApoE locus accounts for less than half the genetic variation in susceptibility to AD, and at least four other genes are thought to underlie the remaining risk.

As well as the intracellular formation of neurofibrillary tangles, AD is characterised by the extracellular deposition of β-amyloid (Aβ) in senile plaques. Aβ is cleaved sequentially by β- and then γ-secretase. However, the formation of Aβ is a relatively rare occurrence in normal brain; more typically APP is cleaved first by α-secretase rather than β-secretase, thus precluding Aβ production. Recently, it has been shown that α-secretase activity is decreased in the temporal cortex of Alzheimer's patients compared with controls. Given the proteolytic activity of at least six members of the ADAM family of proteins, it may be that members of this family have a role to play in AD. However, which, if any, given that none have been shown, as yet, to have α-secretase activity is unknown. Our own studies have focused on ADAM 12. We have examined this gene to identify any DNA sequence variants which may be genetically associated with Alzheimer's disease. As a result of our investigations we have been able to identify a number of variants (Single Nucleotide Polymorphisms) SNPs of the ADAM 12 gene in individuals suffering from Alzheimer's disease.

Close localisation of disease causing genes may be accomplished by the detection of associations between particular alleles and the disease phenotype. Over short segments of DNA, distinctive alleles of the individual polymorphisms will show non-random association with alleles of neighbouring polymorphisms. This phenomenon, known as “linkage disequilibrium” typically occurs over 50-500 Kilobases (Kb) of DNA and associations between polymorphism and disease are in general unlikely to extend beyond 500 Kb. Linkage disequilibrium may be detected by the study of individuals and by the study of families. Disease causing alleles will be in linkage disequilibrium with non-functional polymorphisms from the same chromosomal segment. It is therefore possible to detect allelic association with disease from particular chromosomal segments, without identifying the exact polymorphism and gene underlying the disease state.

The detection of allelic association may therefore give information as to disease susceptibility in a particular individual. Furthermore, allelic association is indicative of a disease-causing gene being present within a limited distance of DNA in either direction from the allele. Identification of the disease causing gene will allow the identification of individuals at risk of ADAM12-mediated disease, and in particular, LOAD, with the potential for prevention or attenuation of disease. Knowledge of the gene and its activity will enable predictions to be made regarding the type of disease and the clinical course of disease or the response to particular treatments. This diagnostic information will be of use to the health care, pharmaceutical and insurance industries.

### SUMMARY OF THE INVENTION

Accordingly, the present invention concerns an isolated nucleic acid molecule that encodes the ADAM 12 protein, or a functional part thereof, and which further comprises any one or more of the following variants:
1. g57690 C>G
2. g109183 G>A
3. g232794 A>G
4. g232959 C>T
5. g259753 A>G
6. g271466 G>C
7. g287117 T>C
8. g289573 A>G
9. g289746 C>T
10. g309989 G>A
11. g312567 C>T
12. g313352 G>A
13. g313746 delT
14. g316406 A>G
15. g317381 C>T
16. g323237 C>T
17. g323327 G>A
18. g323354 A>G
19. g323362 G>A
20. g323441 C>T
21. g338785 C>T
22. g341999 T>C
23. g345179 C>T
24. g345180 A>G
25. g345518 T>G
26. g345987 G>A
27. g346355 C>T
28. g346571 T>A
29. g366613 A>G
30. g371311 C>T
31. g371348 G>A
32. g371435 G>C

In a preferred embodiment of the invention the variants are markers or indicators of Alzheimer's Disease and, in particular, Late Onset Alzheimer's Disease.

According to a further aspect, the present invention concerns an isolated nucleic acid molecule that encodes the ADAM12 protein, or a functional part thereof, and which further comprises any one or more of the following Alzheimer's Disease associated variants:
5. g259753 A>G
6. g271466 G>C
7. g287117 T>C
8. g289573 A>G
10. g309989 G>A
11. g312567 C>T
12. g313352 G>A
13. g313746 delt
14. g316406 A>G
15. g317381 C>T
16. g323237 C>T
20. g323441 C>T
21. g338785 C>T
22. g341999 T>C
25. g345518 T>G
29. g366613 A>G
30. g371311 C>T
31. g371348 G>A
32. g371435 G>C

In a preferred embodiment of the invention the said variants further comprise:
1. g57690 C>G
2. g109183 G>A
3. g232794 A>G
4. g232959 C>T
9. g289746 C>T
17. g323327 G>A
18. g323354 A>G
19. g323362 G>A
23. g345179 C>T
24. g345180 A>G
26. g345987 G>A
27. g346355 C>T
28. g346571 T>A

More preferably, the invention concerns an isolated nucleic acid molecule including any one or more of the aforementioned variants which is in linkage disequilibrium with a further polymorphism. More preferably still, said further polymorphism is one or more of the polymorphisms shown in Table 2A. Accordingly, the invention further comprises an isolated nucleic acid molecule which comprises any of the LD variants shown in Table 2A.

In a further preferred embodiment of the invention said isolated nucleic acid molecule comprises a plurality of any of the aforementioned variants.

According to a further aspect of the invention there is provided a polypeptide or protein encoded by any one of the aforementioned isolated nucleic acid molecules. In the instance where the polypeptide or protein is encoded by SNPs 1 (g57690) or 2 (g109183), the protein comprises the following, respective, sequence changes 1 (R48G) or 2 (R71Q).

According to a yet further aspect of the invention there is provided a method for diagnosing the existence of, or susceptibility to, ADAM12-mediated disease including, but not limited to, cancers, disorders of myogenesis, adipogenesis, cardiac hypertrophy and Alzheimer's Disease and in particular Late Onset Alzheimer's Disease (LOAD) comprising:
a) obtaining a sample of nucleic acid encoding the protein ADAM 12, or a sample of said protein, from an individual to be tested;
b) examining said nucleic acid, or said protein, for any one or more of the variants described herein, or a corresponding variant in said protein, or a polymorphism in linkage disequilibrium therewith as described herein; and
c) where one or more of said variants and/or polymorphisms exists determining that the individual may be suffering from, or susceptible to, an ADAM12-mediated disease.

In a preferred embodiment of the invention said method is undertaken to determine the existence of, or susceptibility to, Alzheimer's Disease and in particular Late Onset Alzheimer's Disease.

In a preferred diagnostic method of the invention said sample is examined for more than one of said variants and in particular a plurality of the following variants represented by SNPs 5-8,10-16,20-22,25,29-32.

More preferably still at least one selected Haplotype of said isolated nucleic acid sample is/are examined to determine if a plurality of the variants described herein are present. The selected Haplotype(s) for performing this method of diagnosis is/are listed in Table 2B and comprises Haplotype(s) characterised by the following SNPs: 1_{—}2_{—}8, 1_{—}2_{—}16, 1_{—}2_{—}22, 1_{—}2_{—}31, 1_{—}8_{—}16, 1_{—}8_{—}22, 1_{—}8_{—}31, 1_{—}16_{—}22, 1_{—}16_{—}31, 1_{—}22_{—}31, 2_{—}8_{—}16, 2_{—}8_{—}22, 2_{—}8_{—}31, 2_{—}16_{—}22, 2_{—}16_{—}31, 2_{—}22_{—}31, 8_{—}16_{—}22, 8_{—}16_{—}31, 8_{—}22_{—}31, 16_{—}22_{—}31

In a preferred embodiment of the invention said isolated nucleic acid molecule is gDNA.

In an alternative embodiment of the invention said isolated nucleic acid molecule is cDNA and the diagnostic method, ideally, involves identifying one or more of the variants represented by SNPs 1,2,16,17, 21 and 26.

In yet an alternative or additional method of the invention the sample is a polypeptide or protein sample and the method involves identifying the following polypeptide or protein sequence changes R48G or R71Q.

It will be apparent that conventional means can be used for performing the diagnostic method of the invention. Typically, the variants will be identified using primers and genotyping technology.

DNA Amplification

For amplification purposes, pairs of primers are provided. These include a 5′primer, which hybridises to the 5′end of the nucleic acid sequence to be amplified, and a 3′primer, which hybridises to the complementary strand of the 3′end of the nucleic acid to be amplified. Preferred primers are those listed in

Table 1C.

Probes and primers may be labelled, for example to enable their detection. Suitable labels include for example, a radiolabel, enzyme label, fluoro-label, and biotin-avidin label for subsequent visualisation in, for example, a southern blot procedure. A labelled probe or primer may be reacted with a sample DNA or RNA, and the areas of the DNA or RNA which carry complementary sequences will hybridise to the probe, and become labelled themselves. The labelled areas may be visualised, for example by autoradiography.

Preferably, the probes and/or primers hybridise under “stringent conditions”, which refers to the washing conditions used in a hybridisation protocol. The hybridisation conditions for probes are preferably sufficiently stringent to allow distinction between different alleles of a polymorphism upon binding of the probes. In general, the washing conditions should be combination of temperature and salt concentration so that the denaturation temperature is approximately 5 to 20° C. below the calculated Tm of the nucleic acid under study. The Tm of a nucleic acid probe of 20 bases or less is calculated under standard conditions (1M NaCl) as [4 C×(G+C)+2 C×(A+T)], according to Wallace rules for short oligonucleotides. For longer DNA fragments, the nearest neighbour method, which combines solid thermodynamics and experimental data may be used, according to the principles set out in Breslauer et al., PNAS 83: 3746-3750 (1986). The optimum salt and temperature conditions for hybridisation may be readily determined in preliminary experiments in which DNA samples immobilised on filters are hybridised to the probe of interest and then washed under conditions of different stringencies. While the conditions for PCR may differ from the standard conditions, the Tm may be used as a guide for the expected relative stability of the primers. For short primers of approximately 14 nucleotides, low annealing temperatures of around 44° C. to 50° C. are used. The temperature may be higher depending upon the base composition of the primer sequence used. Typically, the salt concentration is no more than 1M, and the temperature is at least 25° C. Suitable conditions are 5×SSPE (750 mM NaCl, 50 mM NaPhosphate, 5 mM EDTA pH 7.4) and a temperature of 25-30° C.

Genotyping

Any technique, including those known to persons skilled in the art, may be used in the above method. These may include the use of probes or primers as described. Preferably, the method comprises first removing a sample from a subject. More preferably, the method comprises isolating from a sample a nucleic acid.

In particular, methods for use in this aspect include those known to persons skilled in the art for identifying differences between nucleic acid sequences, for example direct probing, allele specific hybridisation, PCR methodology including Pyrosequencing (Ahmadian A, Gharizadeh B, Gustafsson AC, Sterky F, Nyren P, Uhlen M, Lundeberg J. Single-nucleotide polymorphism analysis by pyrosequencing, Anal Biochem. 2000 Apr. 10; 280(1)103-10; Nordstrom T, Ronaghi M, Forsberg L, de Faire U, Morgenstern R, Nyren P. Direct analysis of single-nucleotide polymorphism on double-stranded DNA by pyrosequencing. Biotechnol Appl Biochem. 2000 Apr.; 31 (Pt 2): 107-12) Allele Specific Amplification (ASA) (W093/22456), Allele Specific Hybridisation, Single Base Extension (U.S. Pat. No. 4,656,127), ARMS-PCR, Taqman™ (U.S. Pat. Nos. 4683202; 4683195; and 4965188), Oligo-ligation assays, Single-strand Conformational analysis ((SSCP) Orita et al PNAS 86 2766-2770 (1989)), Genetic Bit Analysis (WO 92/15712), RFLP analysis, direct DNA sequencing, mass spectrometry (MALDI-TOF) and DNA arrays. The appropriate restriction enzyme, will, of course, be dependent upon the polymorphism and restriction site, and will include those known to persons skilled in the art. Analysis of the digested fragments may be performed using any method in the art, for example gel analysis, or Southern blots.

According to a further aspect of the invention there are provided variants of ADAM12 gene or protein useful for the diagnosis of, or susceptibility to, ADAM12-mediated diseases including, but not limited to, cancers, disorders of myogenesis, adipogenesis, cardiac hypertrophy and more preferably Alzheimer's Disease and in particular Late Onset Alzheimer's Disease.

According to a yet further aspect of the invention there is provided a method of diagnosis of, or susceptibility to, ADAM12-mediated diseases which comprises determining the level of ADAM12, or the activity thereof, in a sample.

Table 1A shows the nucleic acid sequence 50 bp 5′ and 3′ to the variants we have identified. This information is used to design the primers for performing the diagnostic method of the invention.

Accordingly, there is provided an oligonucleotide for identifying a variant in the ADAM 12 gene, which oligonucleotide is complementary to a sequence of nucleotides shown in Table 1A which is either 5′ or 3′ to said variant.

Additionally, or alternatively, there is provided an oligonucleotide for identifying a variant in the ADAM12 gene comprising an oligonucleotide listed in Table 1C.

Most ideally, said oligonucleotide is between 10-20 nucleotides in length.

More preferably still a pair of oligonucleotides are provided to identify one of the variants shown in Table 1A, one of which is complementary to a sequence of nucleic acids 5′ of the variant of interest and the other is complementary to a sequence of nucleic acids 3′ of said variant.

In yet a further aspect of the invention there is provided an associated Haplotype, as shown in Table 2B, or a combination thereof, of an individual suffering from, or susceptible to, Alzheimer's disease. Further there is provided use of this Haplotype, or a combination thereof, for diagnosing the existence of, or susceptibility to, Alzheimer's disease.

Reference herein to Haplotype includes reference to the set of alleles or variants of the ADAM 12 gene that associate with Alzheimer's disease and in particular LOAD.

According to a further aspect of the invention there is provided a kit suitable for carrying out the aforementioned diagnostic methods of the invention which kit comprises:
a) at least one oligonucleotide that is complementary to a region of ADAM 12 gene that comprises, or is adjacent to, any one or more of the following variants:
1. g57690 C>G
2. g109183 G>A
3. g232794 A>G
4. g232959 C>T
5. g259753 A>G
6. g271466 G>C
7. g287117 T>C
8. g289573 A>G
9. g289746 C>T
10. g309989 G>A
11. g312567 C>T
12. g313352 G>A
13. g313746 delT
14. g316406 A>G
15. g317381 C>T
16. g323237 C>T
17. g323327 G>A
18. g323354 A>G
19. g323362 G>A
20. g323441 C>T
21. g338785 C>T
22. g341999 T>C
23. g345179 C>T
24. g345180 A>G
25. g345518 T>G
26. g345987 G>A
27. g346355 C>T
28. g346571 T>A
29. g366613 A>G
30. g371311 C>T
31. g371348 G>A
32. g371435 G>C
   and, optionally,
b) one or more reagents suitable for carrying out PCR for amplifying desired regions of a patient's DNA.

Advantageously, the kit comprises oligonucleotides complementary to a region comprising, or adjacent, any one or more of the variants represented by SNPs 5-8, 10-16, 20-22, 25, or 29-32 and this kit is particularly useful for diagnosing Alzheimer's Disease.

In a preferred method of the invention said at least one oligonucleotide is complementary to a sequence of nucleic acids adjacent a polymorphism that is in linkage disequilibrium with one or more of the aforementioned variants and, ideally a polymorphism shown in Table 2A.

In a preferred kit of the invention there is provided a pair of oligonucleotides for identifying a variant or polymorphism of interest one of which is complementary to a sequence 5′ to said variant, or said polymorphism, and the other is complementary to a sequence 3′ to said variant, or said polymorphism. More preferably said kit comprises a plurality of oligonucleotides or oligonucleotide pairs for identifying a plurality of said variants or said polymorphisms. Useful oligonucleotides are shown in Table 1C.

Most preferably still, the kit comprises oligonucleotides suitable for amplifying regions containing, or adjacent, variants represented by SNPs 5-8, 10-16, 20-22, 25, or 29-32. Ideally, oligonucleotides are provided for amplifying a plurality of said aforementioned variants and/or the Haplotypes shown in Table 2B.

The nucleic acid molecules and polypeptides or proteins of the invention have utility in the identification of therapies for the treatment of Alzheimer's disease. It therefore follows that the insertion of one or more of the afore isolated nucleic acid molecules and/or their corresponding polypeptides or proteins into suitable cells, or cell lines, for subsequent investigation, represent useful working tools for identifying AD therapies.

Therefore according to a further aspect of the invention there is provided a vector comprising an isolated nucleic molecule encoding the ADAM 12 gene which includes any one or more of the aforementioned variants or polymorphisms.

More preferably still said vector is suitable for transforming or transfecting a prokaryotic or eukaryotic cell. Most ideally, said vector is provided with means for ensuring expression of said nucleic acid molecule in said selected cell.

The isolated nucleic acid molecules of the invention may be provided in the form of a vector to enable the in vitro or in vivo expression of the isolated nucleic acid molecules of any of the variants described. Vectors include plasmids, chromosomes, artificial chromosomes and viruses and may be expression vectors, which are capable of expressing nucleic acid sequences in vitro or in vivo, or transformation vectors which are capable of transferring the nucleic acid sequence from one environment to another.

The nucleic acid molecules of the invention may be operably linked to one or more regulatory elements including a promoter.The term regulatory elements includes response elements, consensus sites, methylation sites, locus control regions, post-transcriptional modifications, splice variants, homeoboxes, inducible factors, DNA binding domains, enhancer sequences, initiation codons, secretion signals and, polyA sequences. Regions upstream or downstream of a promoter such as enhancers, which regulate the activity of the promoter, are also regulatory elements.

The vector may also comprise an origin of replication; appropriate restriction sites to enable cloning of inserts adjacent to the polynucleotide molecule; markers, for example antibiotic resistance genes; ribosome binding sites: RNA splice sites and transcription termination regions; polymerisation sites; or any other element, such a secretion signals, which may facilitate the cloning and/or expression of the polynucleotide molecule.

Within a vector the gene may be expressed upstream or downstream of an expressed protein tag such as a histidine tag, V5 epitope tag, green fluorescent protein tag, MHC tag or other such tag known to those skilled in the art. Use of such a tag allows easy localisation, affinity purification and detection of the fusion protein with an antibody to the tag moiety.

Where two or more nucleic acid molecules of the invention are introduced into the same vector, each may be controlled by its own regulatory sequences, or all molecules may be controlled by the same regulatory sequence. In the same manner, each molecule may comprise a 3′polyadenylation site. Examples of suitable vectors will be known to persons skilled in the art and include pBluescript II, lambdaZap, and pCMV-Script (Stratagene Cloning Systems, La Jolla, USA).

Appropriate regulatory elements, in particular promoters, will usually depend upon the host cell into which the expression vector is to be inserted. Where microbial host cells are used, promoters such as lactose promoter system, tryptophan (Trp) promoter system, beta-lactamase promoter system or phage lambda promoter systems are suitable. Where yeast cells are used, preferred promoters include alcohol dehydrogenase I or glycolytic promoters. In mammalian host cells, preferred promoters are those derived from immunoglobulin genes, SV40, Adenovirus, Bovine Papilloma virus. Suitable promoters for use in various host cells would be readily apparent to a person skilled in the art (See, for example, Current Protocols in Molecular Biology Edited by Ausubel et al, published by Wiley). In addition, the regulatory elements may be modified, for example by the addition of further regulatory elements, to achieve a desired expression pattern. These vectors may be used to transform host cells, for example, prokaryotic or eukaryotic cells. These cells may be used in the production of recombinant gene products produced from the isolated nucleic acid molecules of the invention, or in the regulation or analysis of the nucleic acid molecules of the invention. The transformed or transfected host cells form part of the invention. Preferred cells include E. coli, yeast, filamentous fungi, insect cells, mammalian cells, preferably immortalised, such as mouse, human and monkey cell lines and derivatives thereof.

Accordingly, there is provided a host cell transformed or transfected with the aforementioned vector.

Advantageously in cell lines, the ADAM12 polypeptides or proteins may be operably linked to a secretion signal, to assist their secretion from the Golgi apparatus to another part of the cell. Suitable secretion signals can be provided by recombinant vectors such as pSecTag2 (Invitrogen Corporation, Carlsbad, Calif.). Polypeptides or proteins expressed from such vectors are fused at the N-terminus to the murine Ig kappa chain leader sequence. The secretion signal may be linked to the soluble ADAM12 polypeptide sequences using techniques available in the art, including recombinant DNA technology. The polypeptides may be linked to a tag such as a histidine tag, V5 epitope tag, green fluorescent protein tag, MHC tag or other tag known to those skilled in the art or to a carrier molecule known to a person skilled in the art.

According to a yet further aspect of the invention there is provided a recombinant cell line that is engineered to express one or more of the polypeptide or protein products encoded by one or more variants of the invention.

Preferably this cell line is of bacterial, yeast, insect, or mammalian origin.

According to a yet further aspect of the invention there is provided a transgenic, non-human animal which over expresses any one or more the aforementioned variants of the invention.

According to a yet further aspect of the invention there is provided a transgenic non-human animal that is deficient for any one or more of the aforementioned variants of the invention.

Advantageously, there is also provided the use of a protein encoded by any one or more of the aforementioned isolated nucleic acid molecules for the production of an antibody.

The invention in another aspect also therefore includes antibodies specific for any one or more proteins of the invention said antibodies may be polyclonal or monoclonal and are produced using conventional techniques. These antibodies may be useful in immuno-analysis; typically for tagging or marking any of the aforementioned proteins. Additionally, the aforementioned antibodies may also be useful in therapies in order to affect the activity of the corresponding proteins. As previously mentioned, the ADAM12 gene encodes two splice variants: a membrane-bound form designated ADAM12-L and a shorter form designated ADAM12-S which is secreted as a soluble protein. Diagnosis of ADAM12-mediated diseases may involve measuring the ratio of membrane-bound to secreted isoforms of ADAM12 protein. This is, advantageously, undertaken using antibodies that bind the different isoforms of ADAM12, using an ELISA or immunolocalisation technique.

Moreover, additionally, the aforementioned nucleic acids and proteins of the invention may be useful in screening for therapeutically active drugs which can be used to treat Alzheimer's disease or any other disease that is linked to ADAM 12. It therefore follows that a method of the invention comprises exposing any one or more of the aforementioned nucleic acids, or proteins, or fragments thereof containing at least one variant of interest to a candidate drug and then determining if any binding or interaction has taken place wherein binding or interaction is indicative of therapeutic activity. More preferably still, the screening method of the invention involves the use of the cell lines of the invention and so the cell lines constructed using ADAM12 variants described herein.

Accordingly, the invention also concerns the identification of agents that bind or interact with any one or more of the nucleic acid molecules or proteins according to the invention.

Preferably, said agents may act to alter the processing of ADAM12, or the levels of ADAM12 in a sample, or the enzyme activity of ADAM12, or the processing and stability of ADAM12 mRNA.

According to a yet further aspect of the invention there is provided the use of an agent that affects the processing, level or activity of ADAM12 or its mRNA in the manufacture of a medicament for the treatment of ADAM 12-mediated diseases.

More preferably still, said diseases include cancer, disorders of myogenesis, adipogenesis, cardiac hypertrophy and Alzheimer's Disease and in particular Late Onset Alzheimer's Disease.

The present invention will now be illustrated with reference to the following wherein:

FIG. 1 shows the DNA sequence structure of the ADAM12 gene.

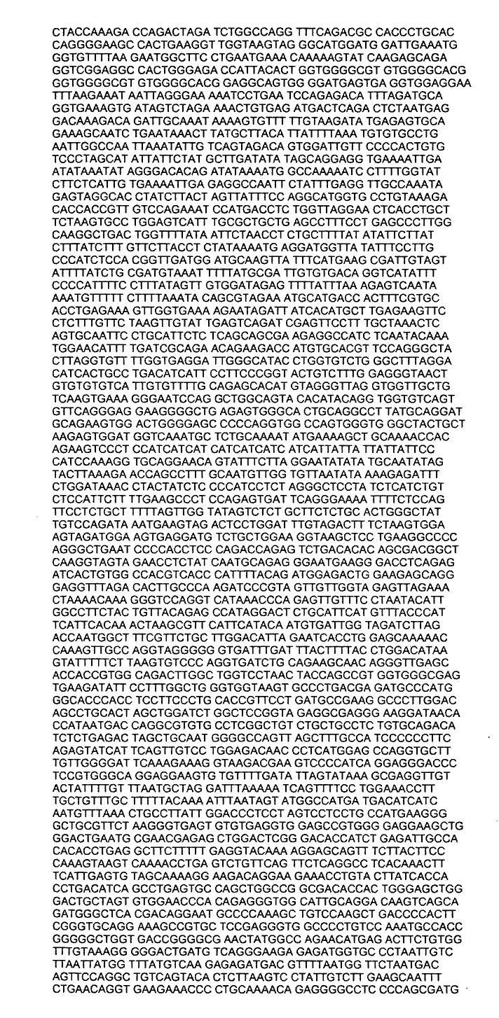

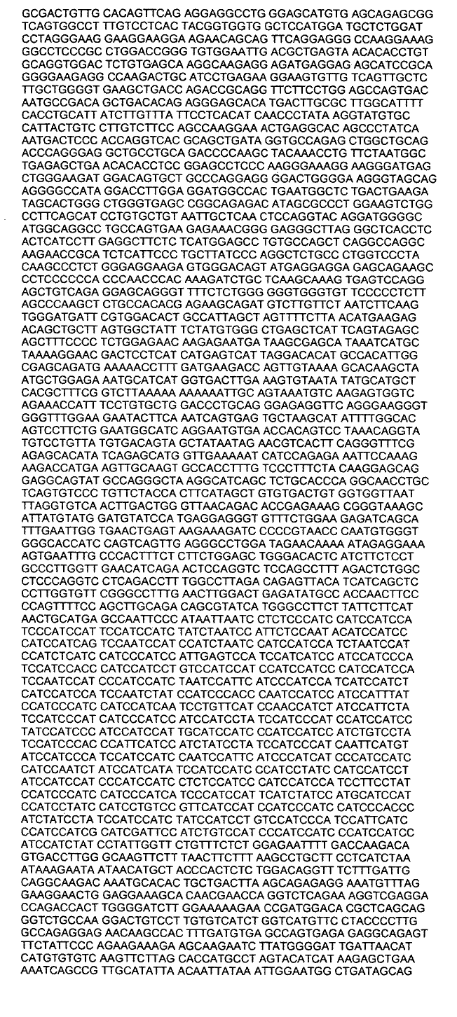

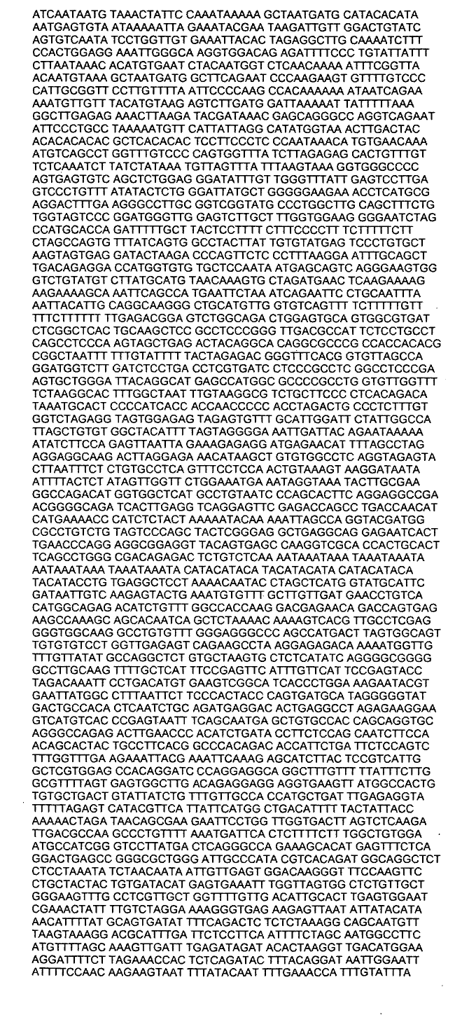

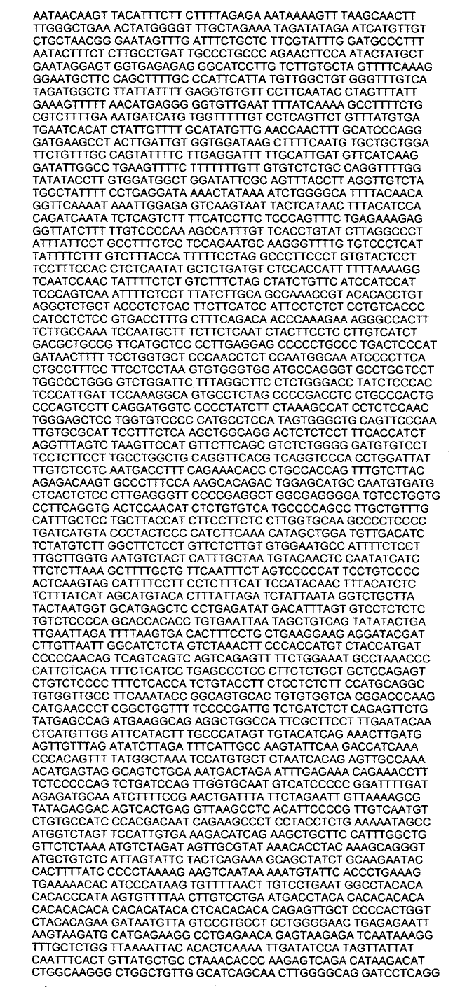

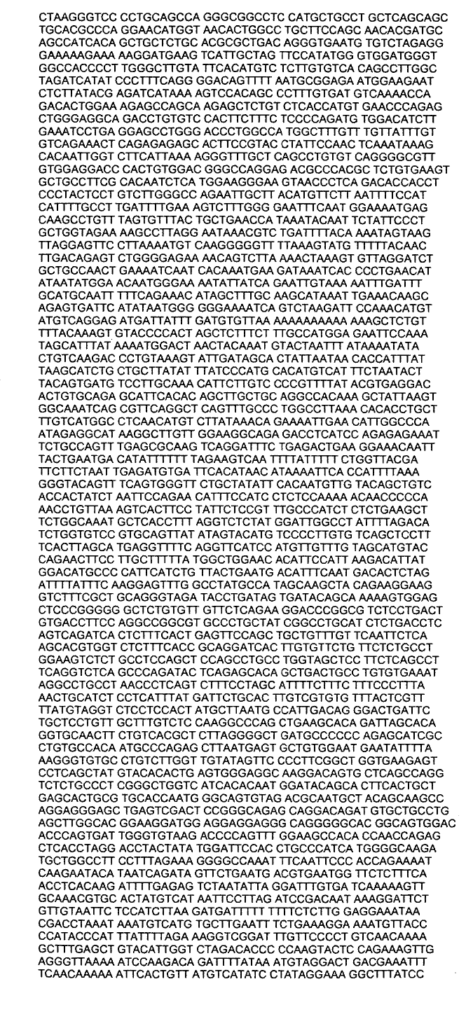

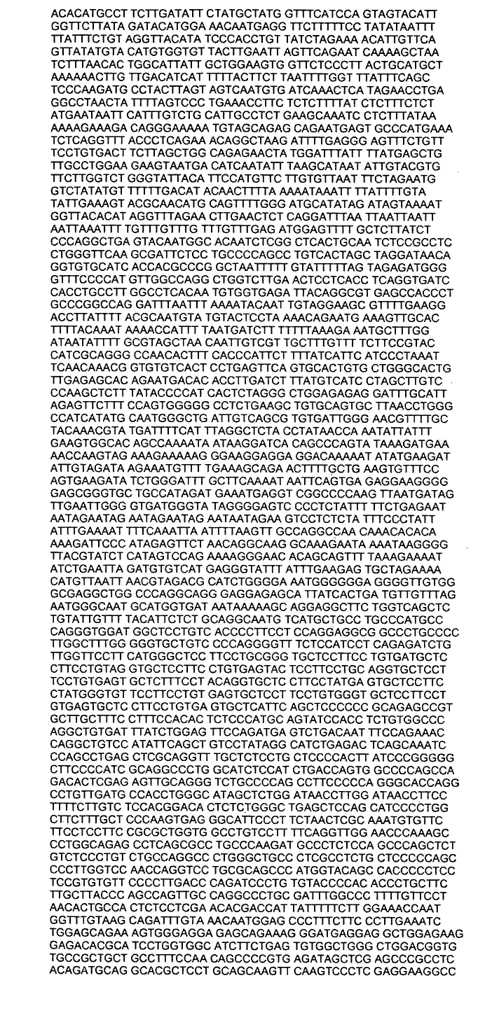

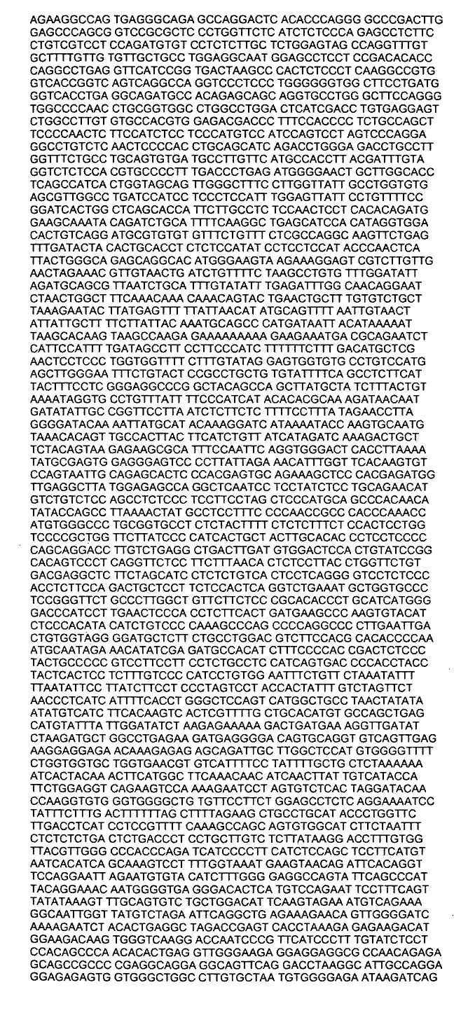

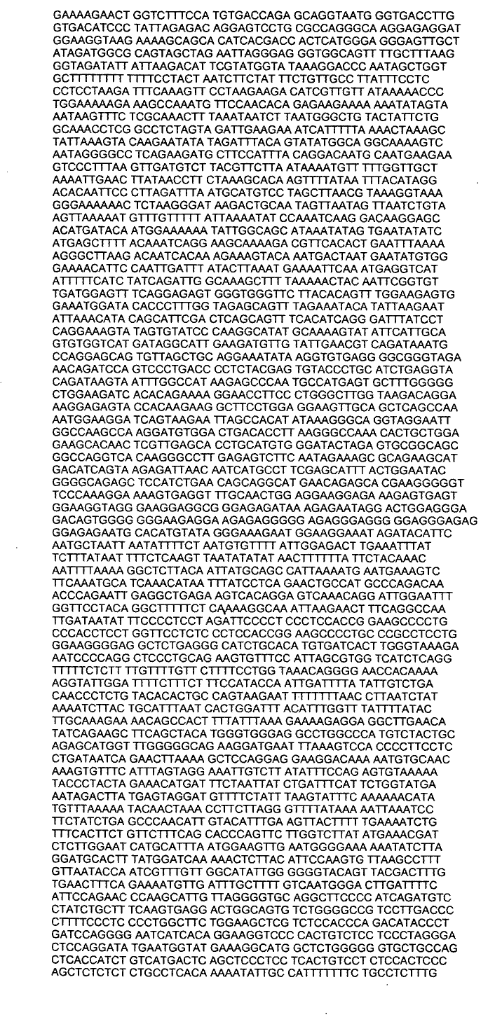

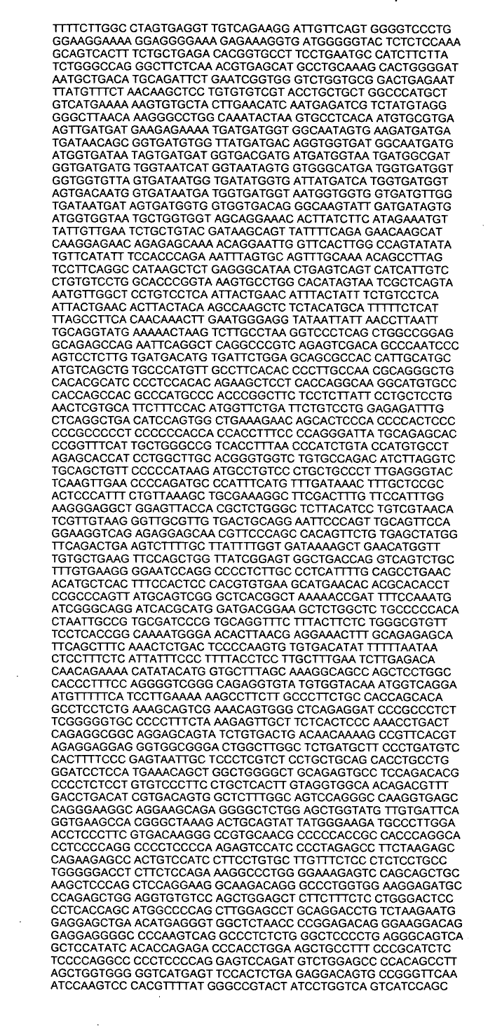

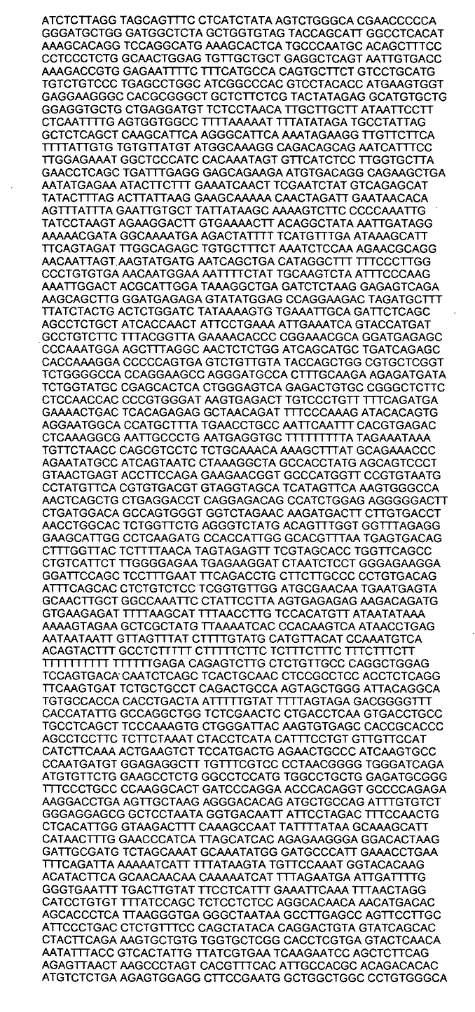

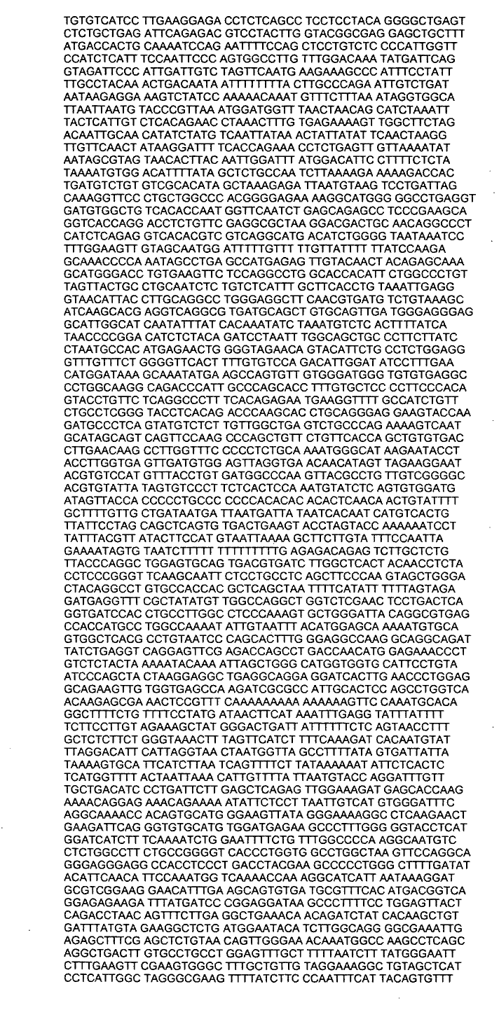

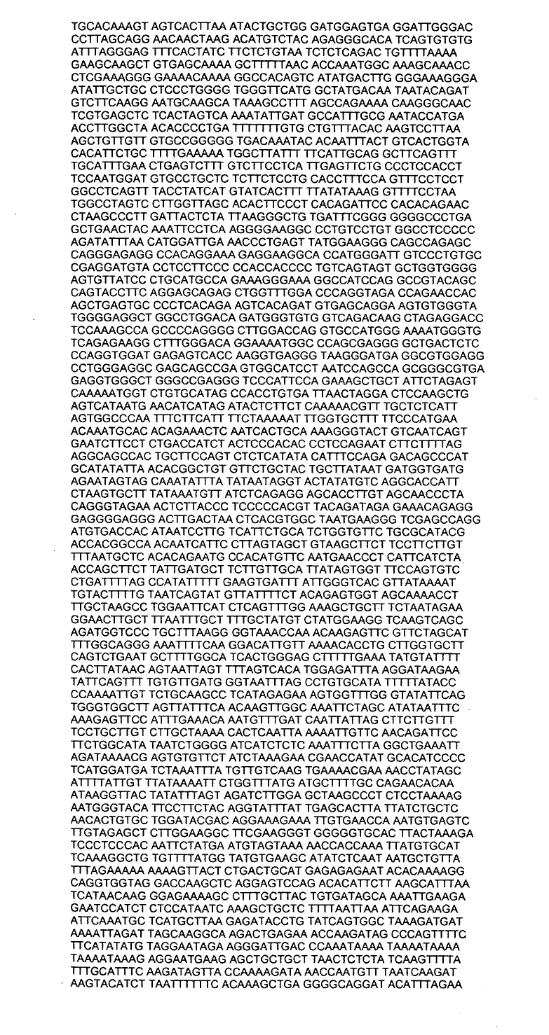

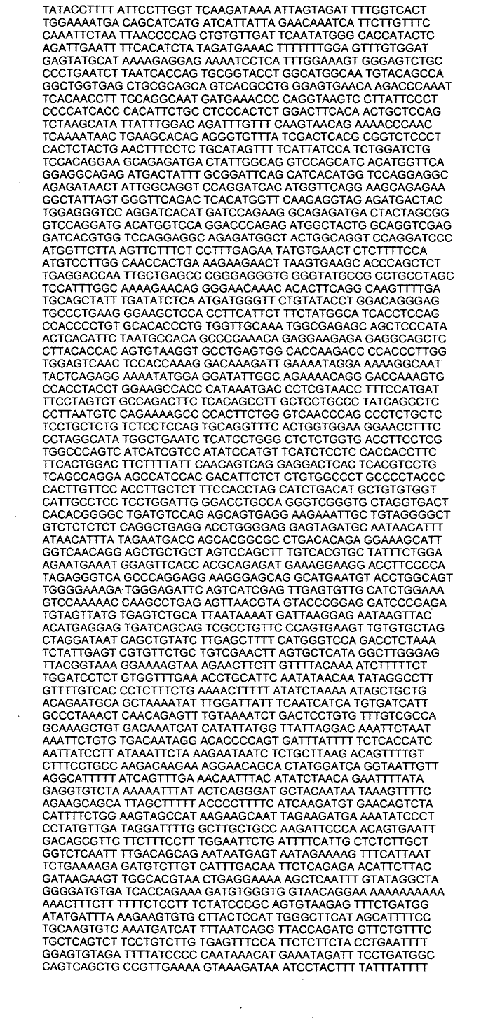

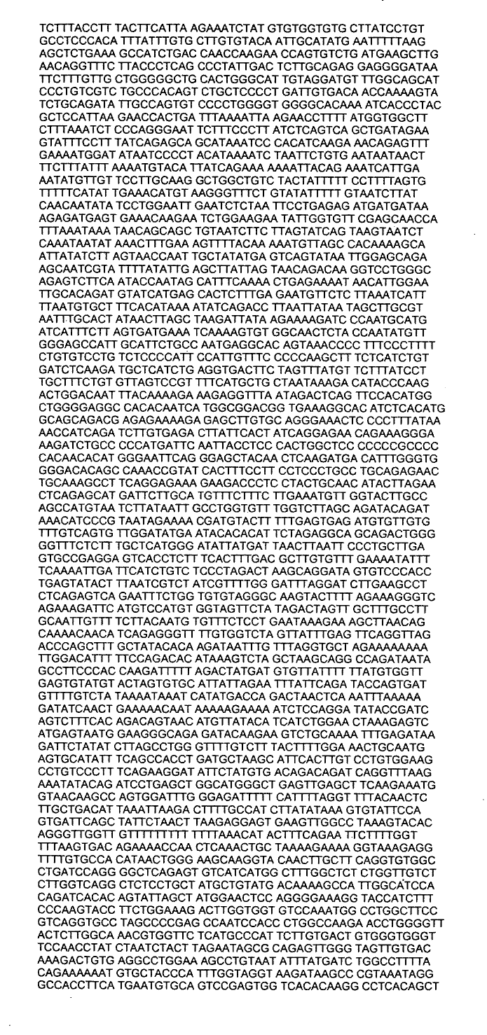

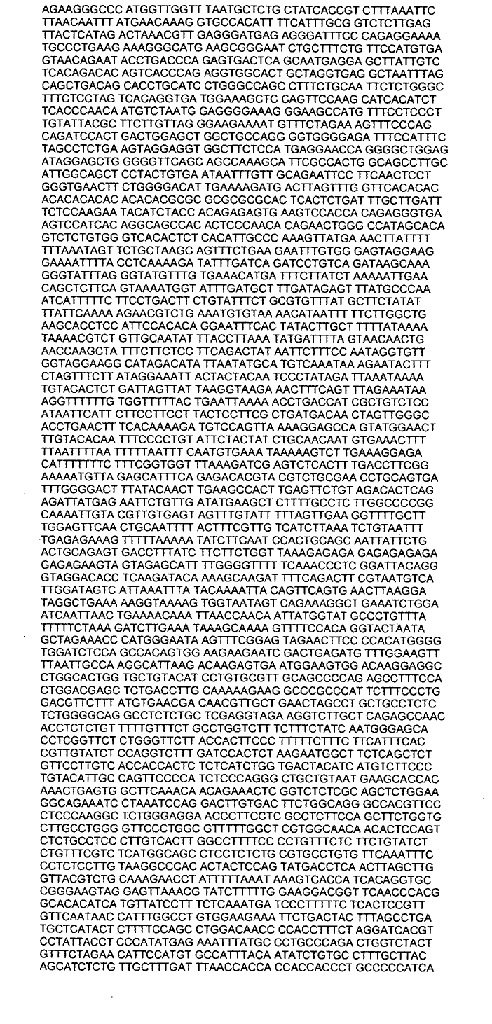

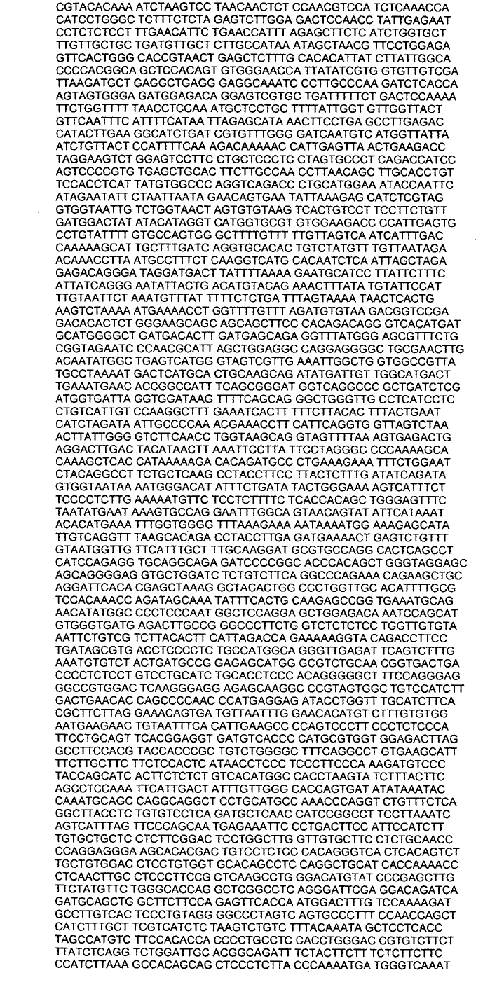

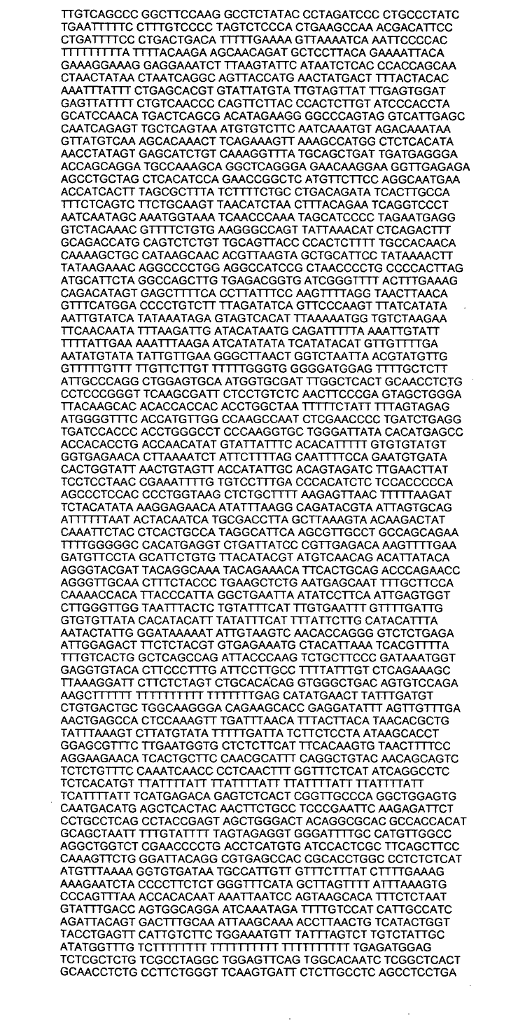

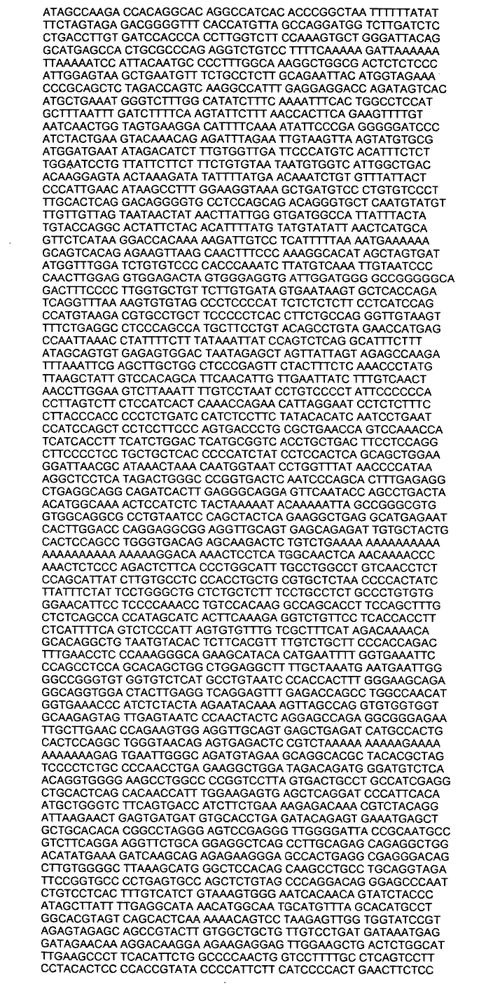

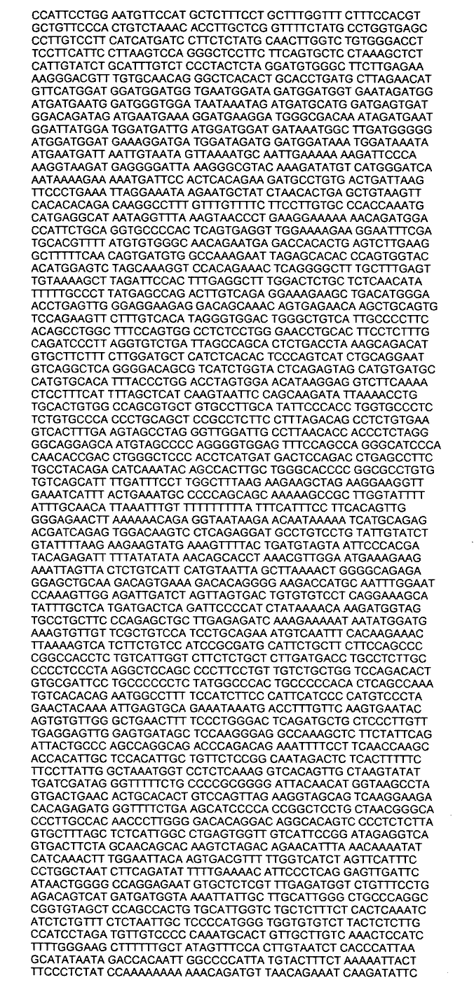

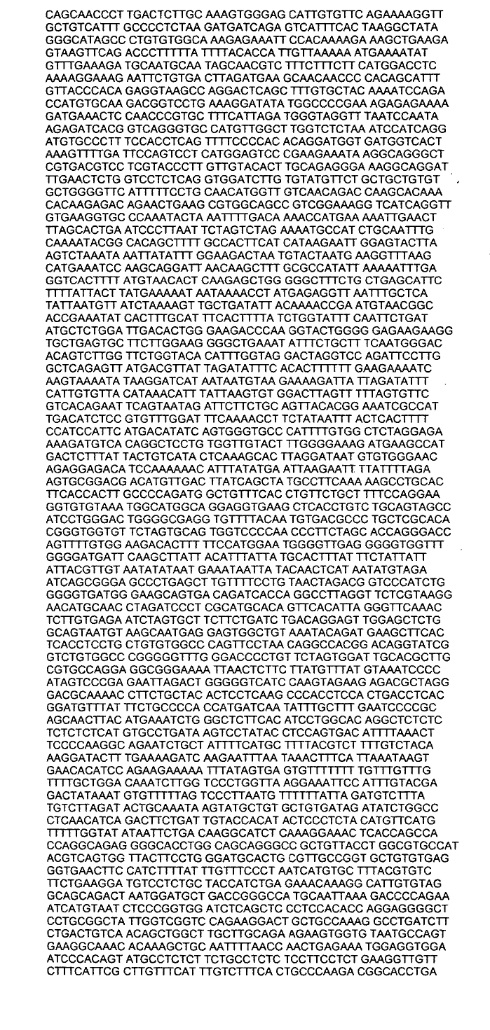

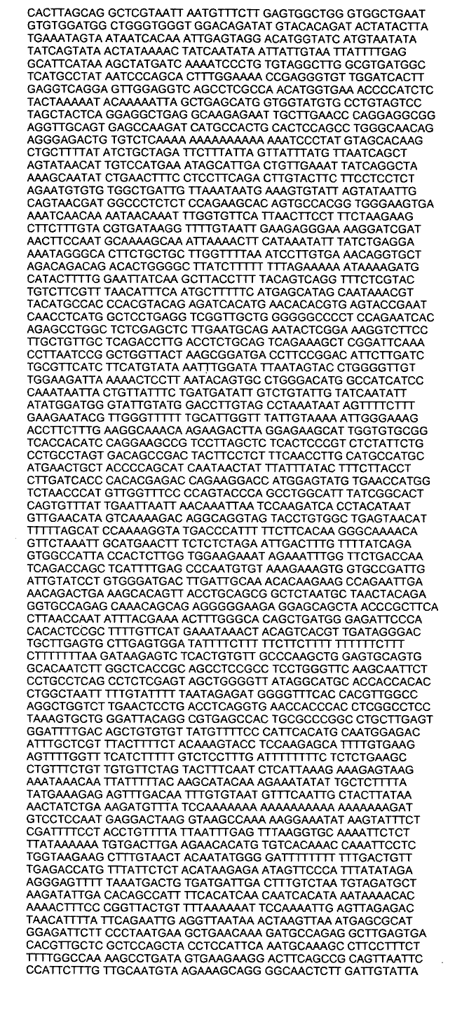

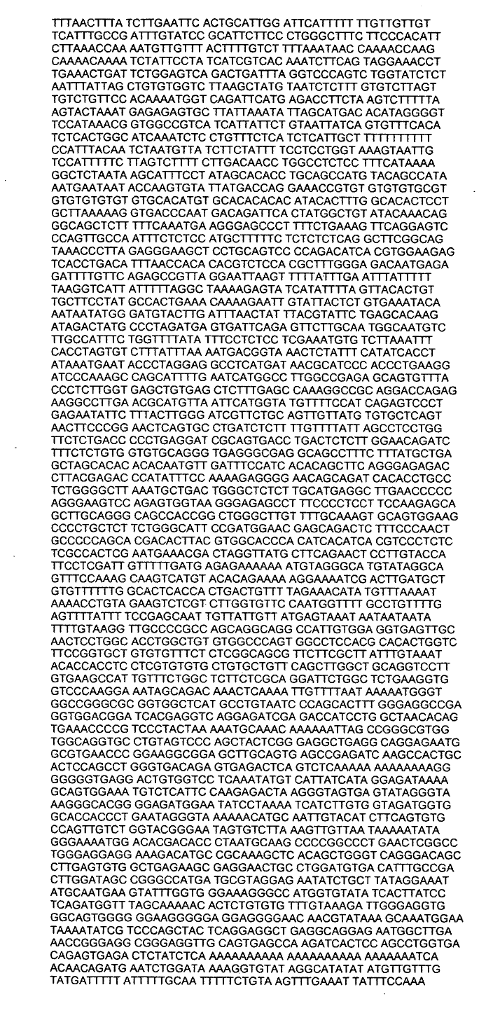

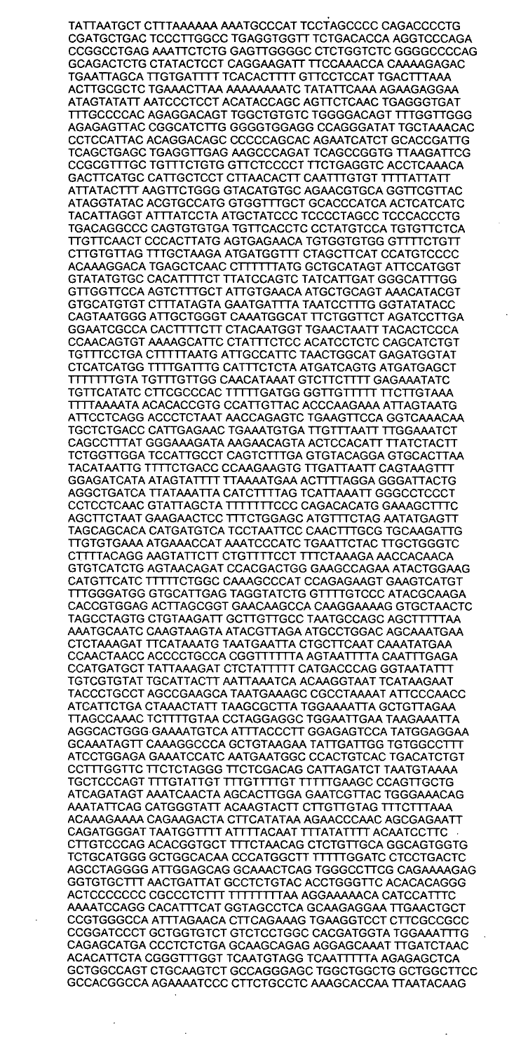

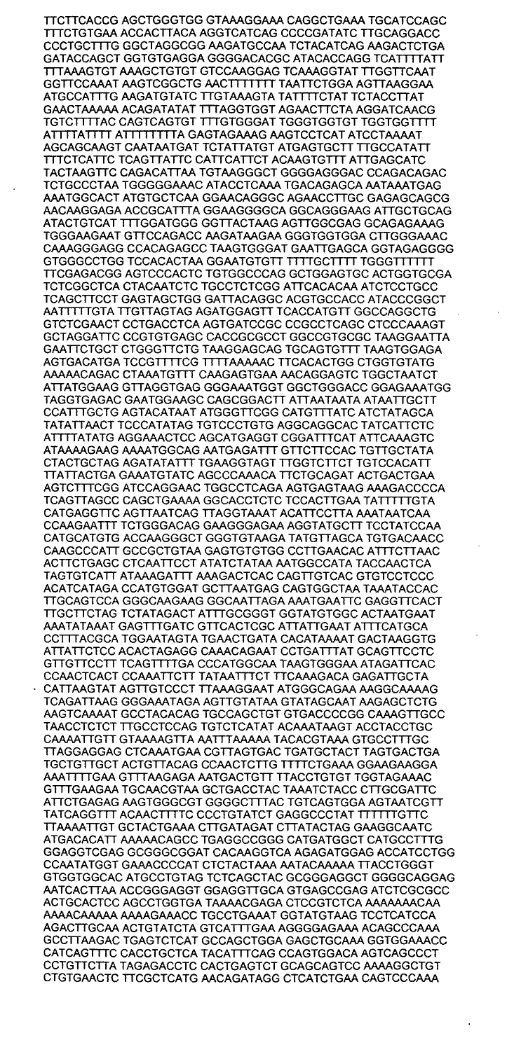

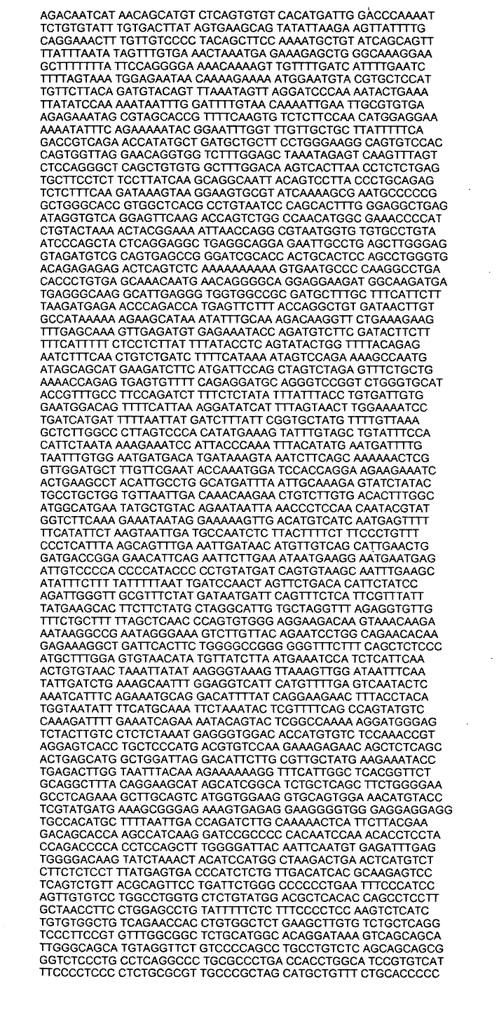

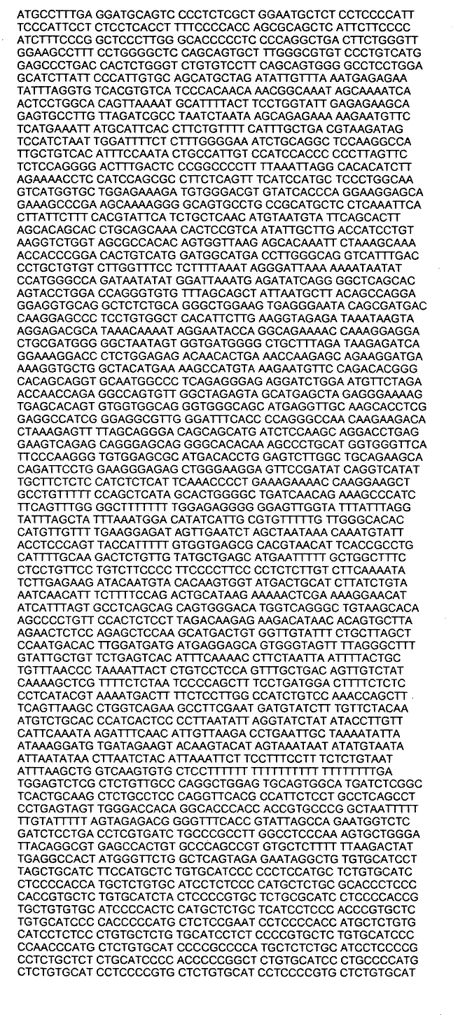

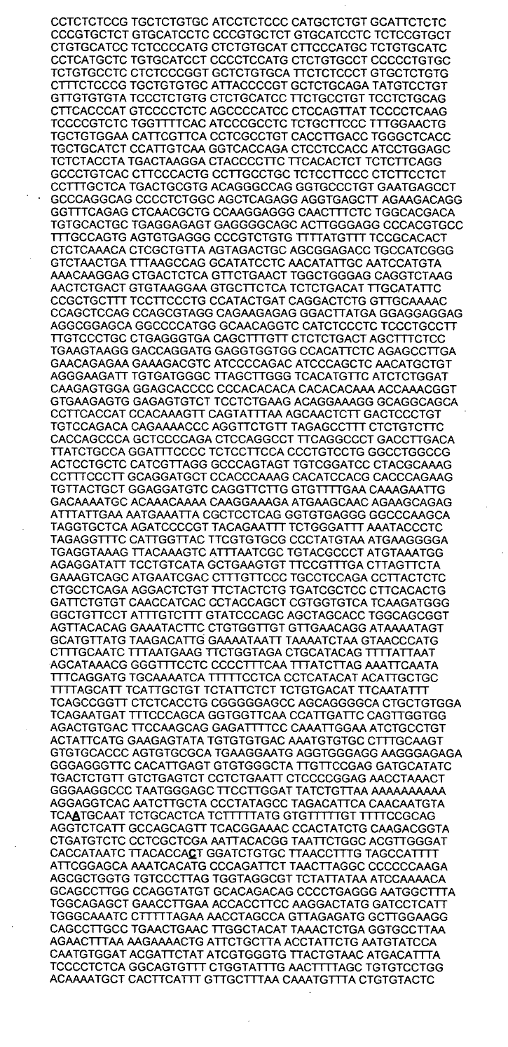

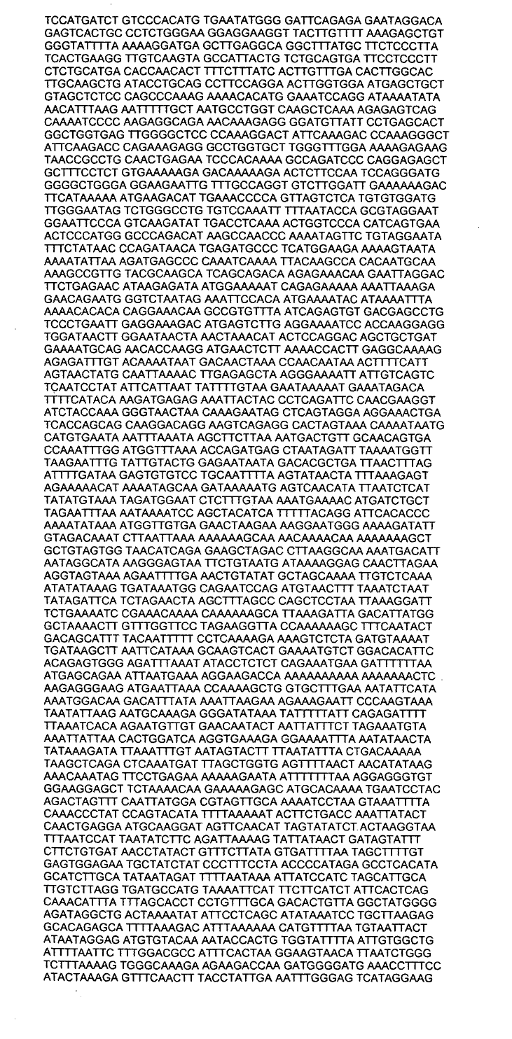

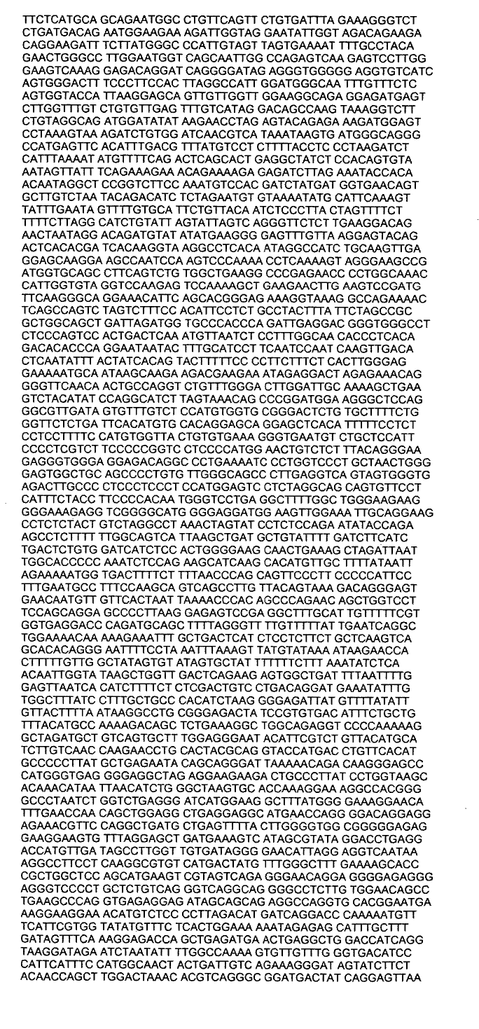

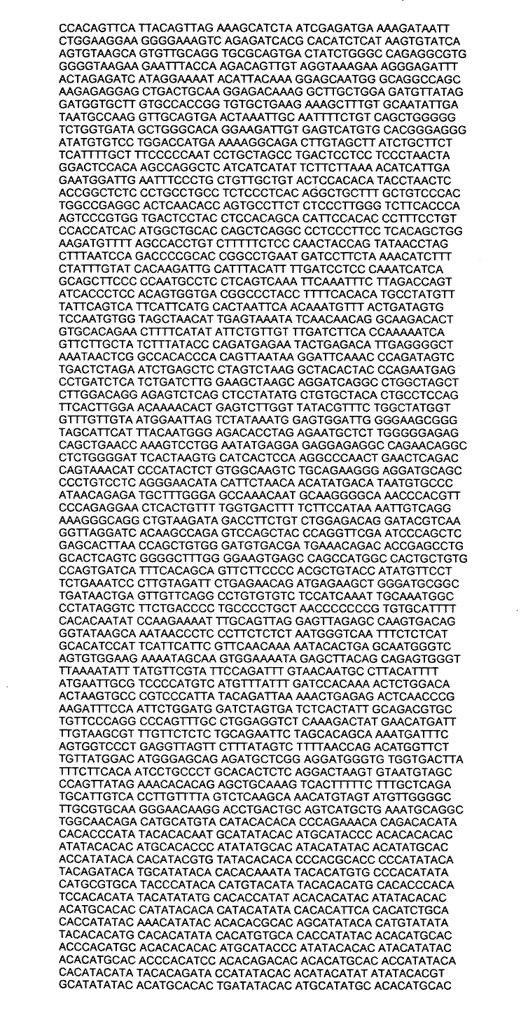

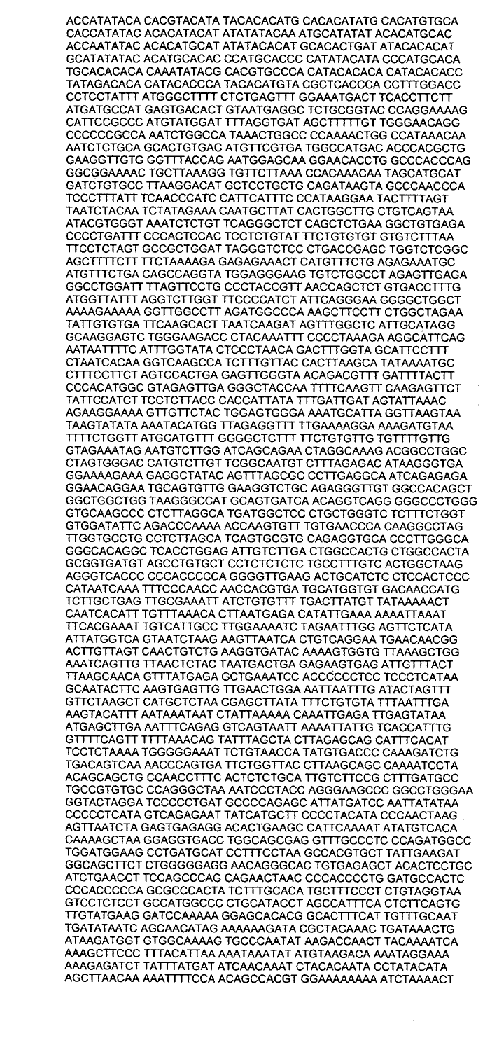

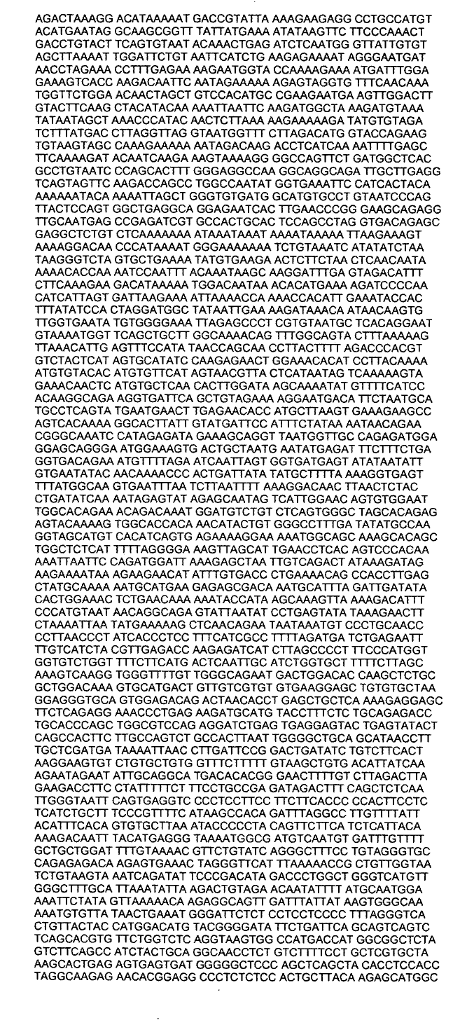

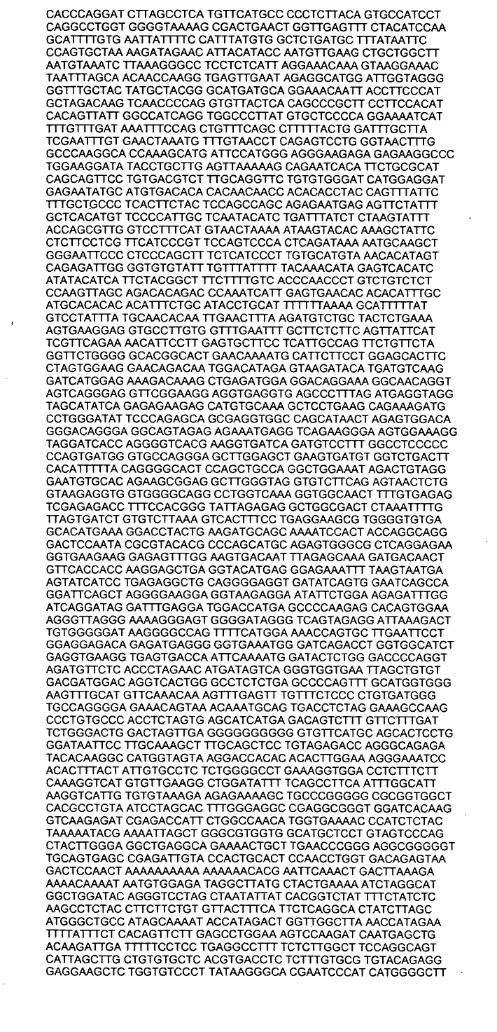

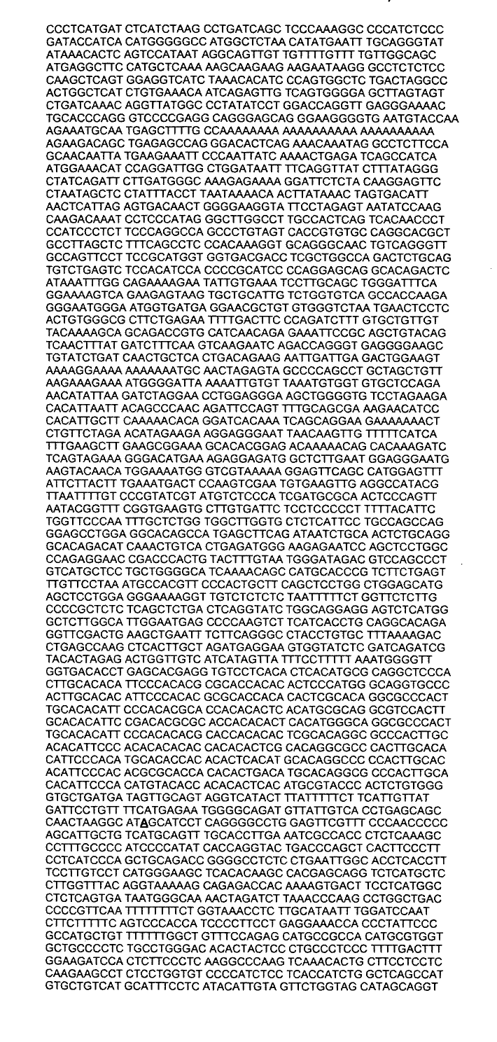

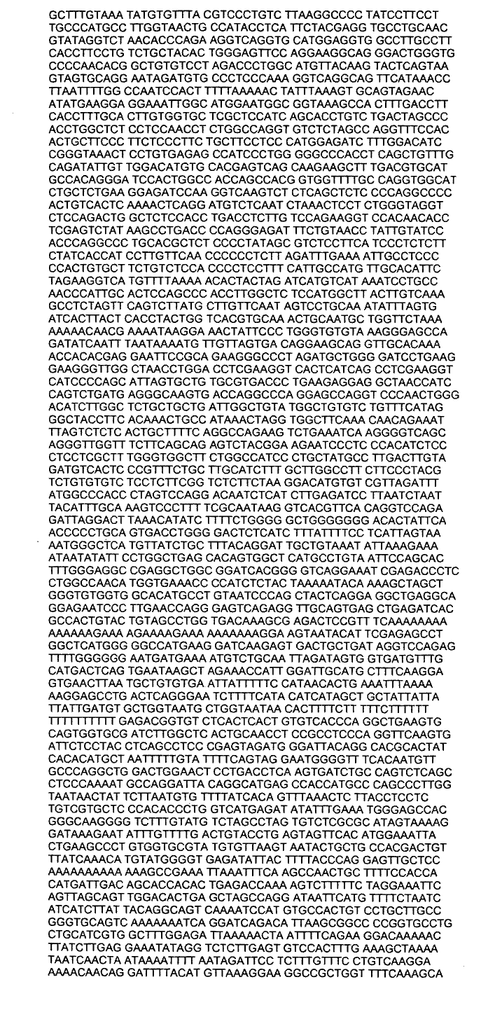

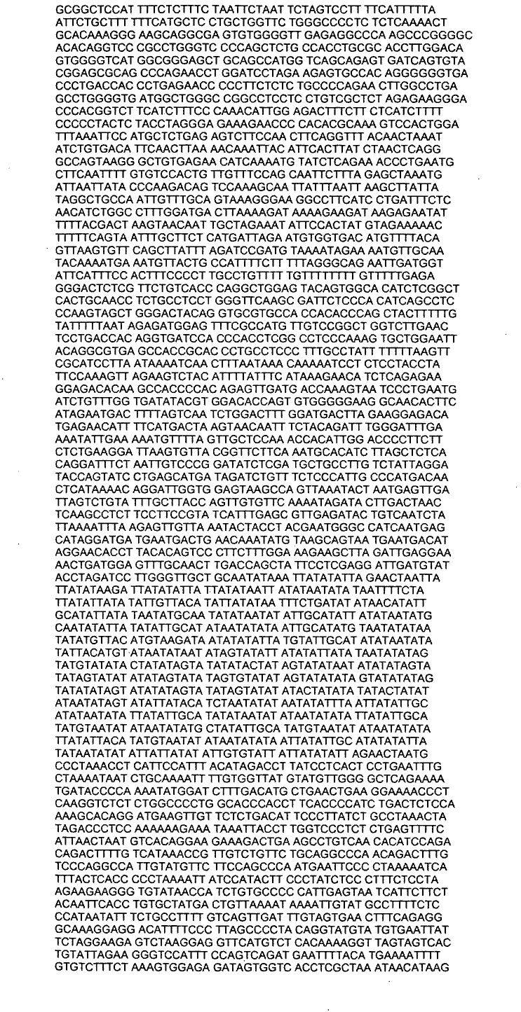

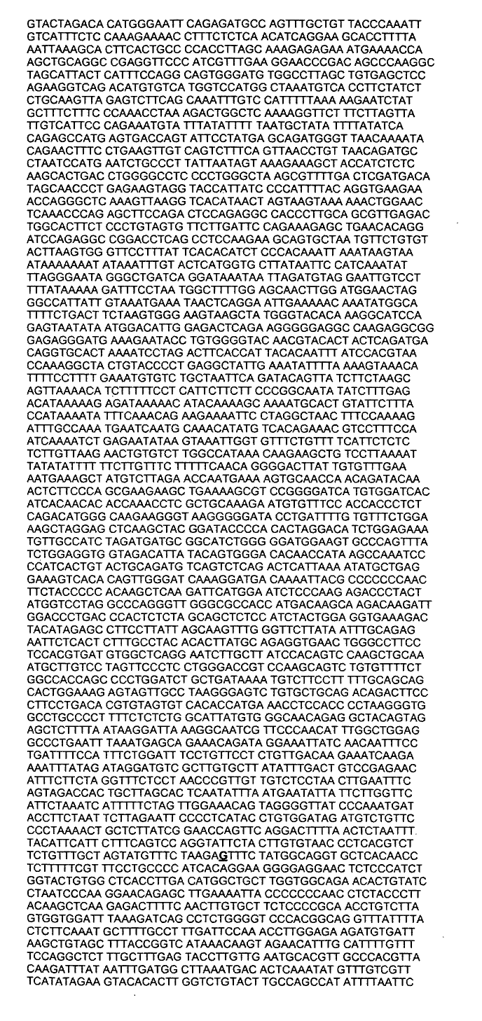

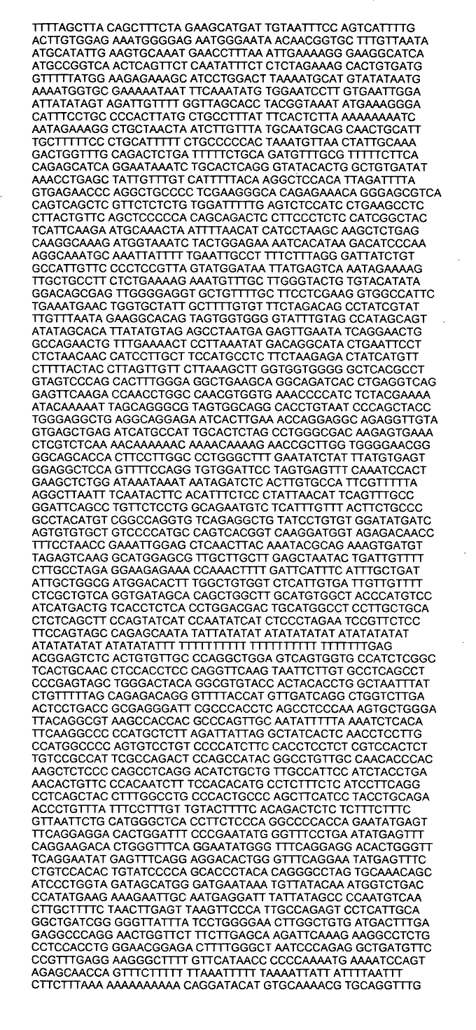

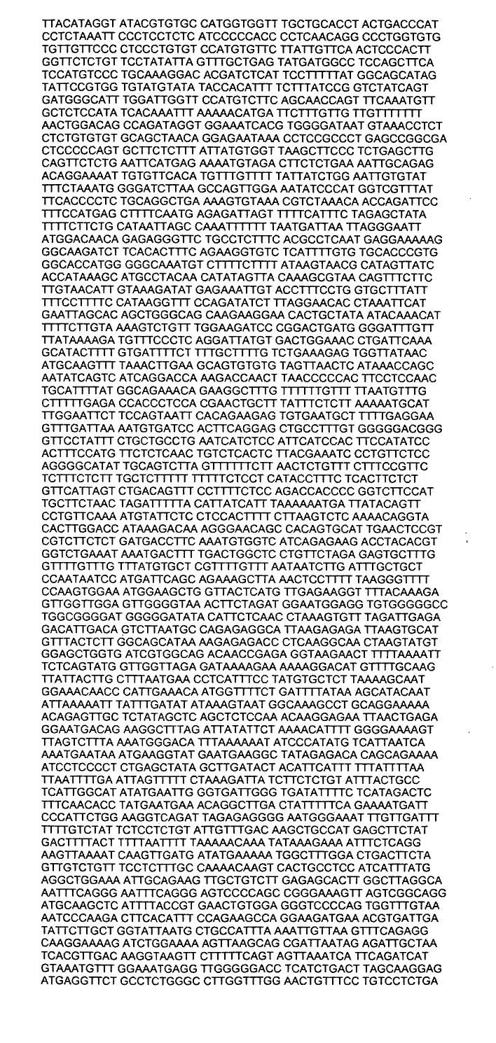

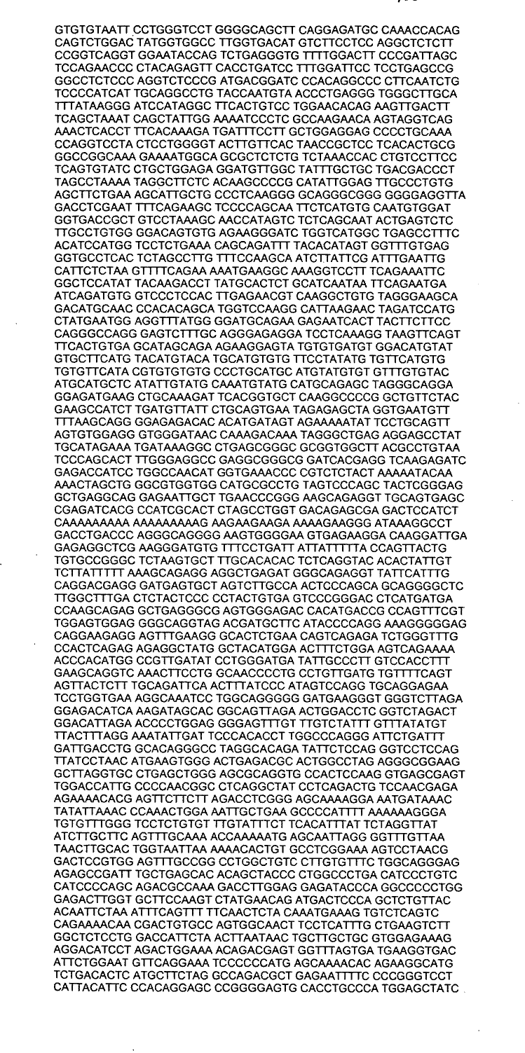

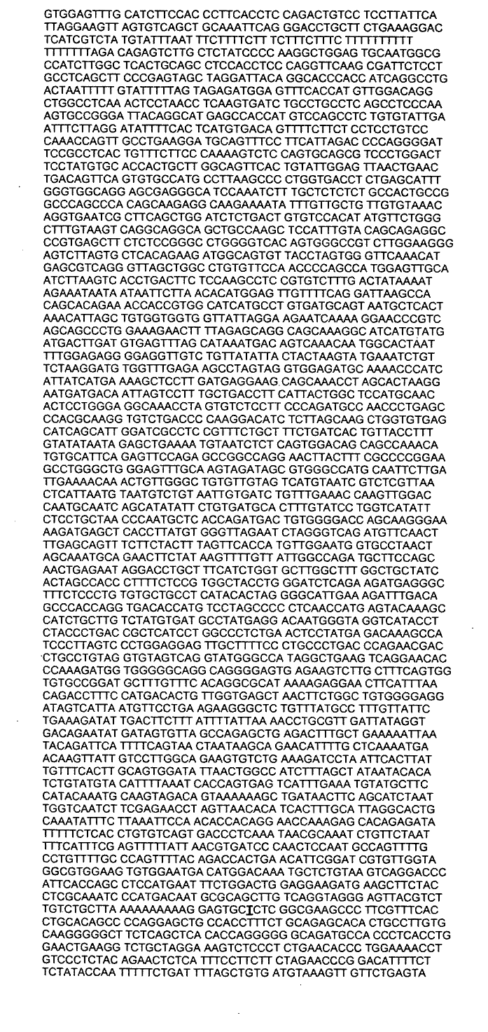

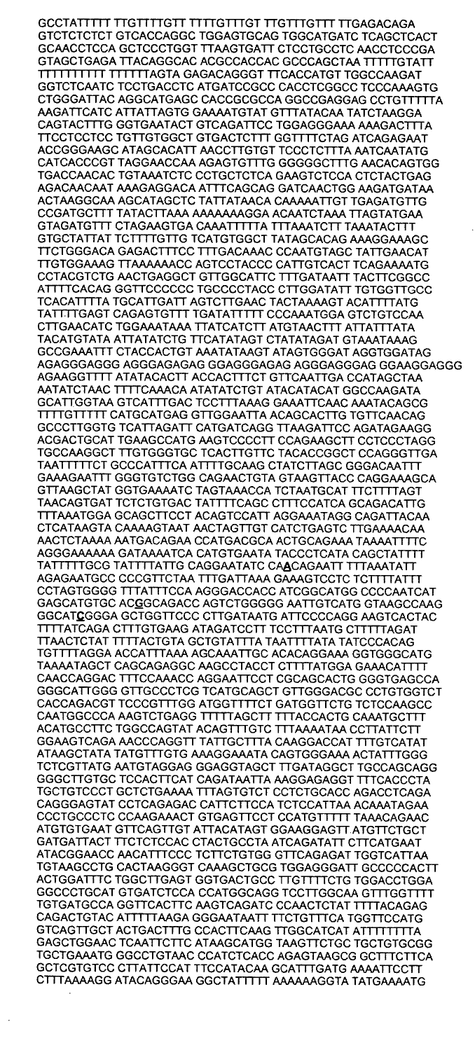

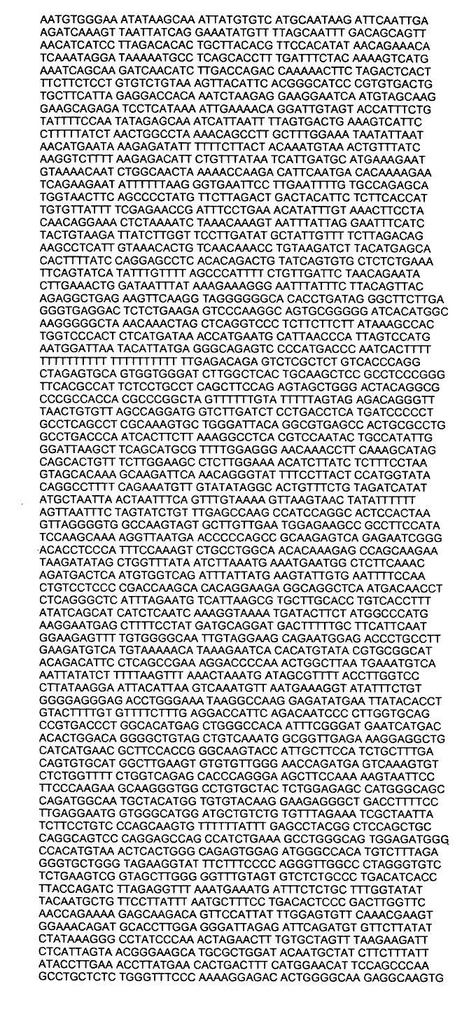

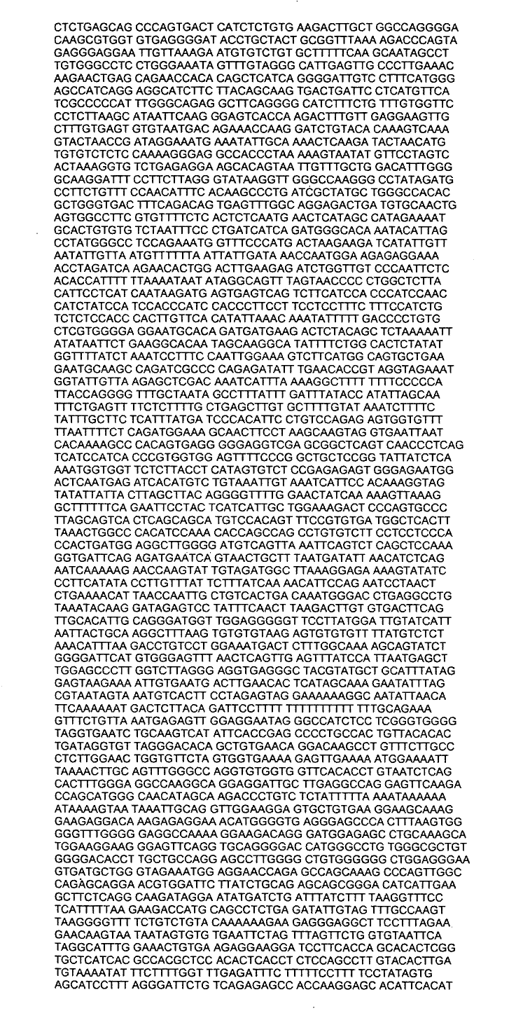

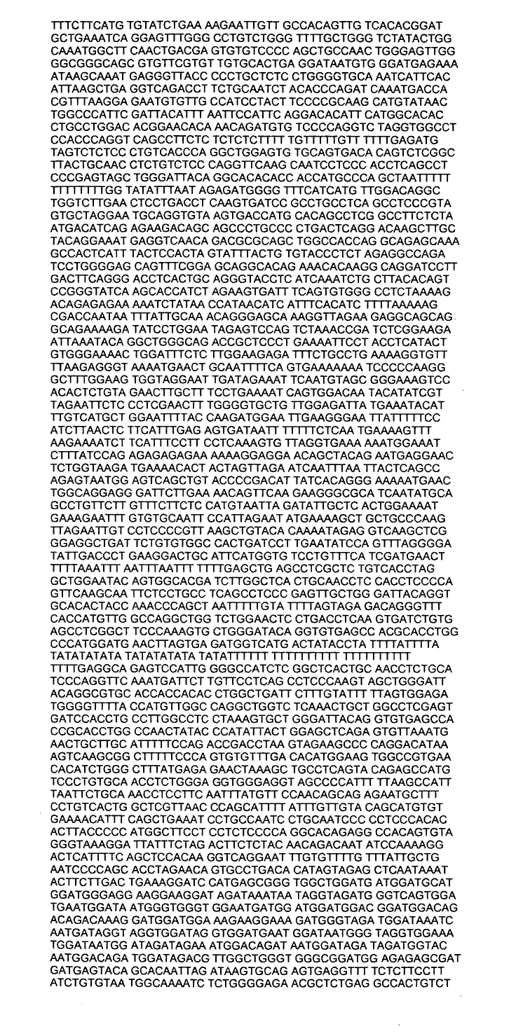

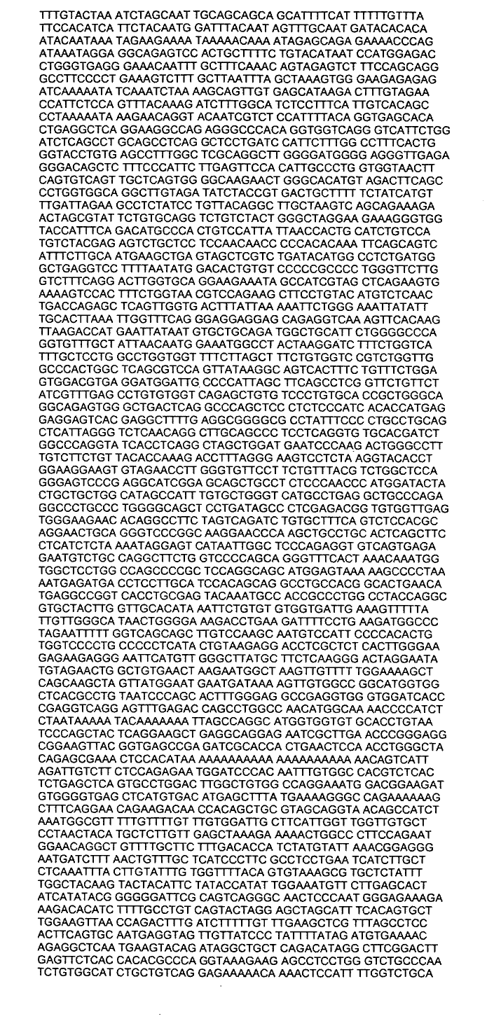

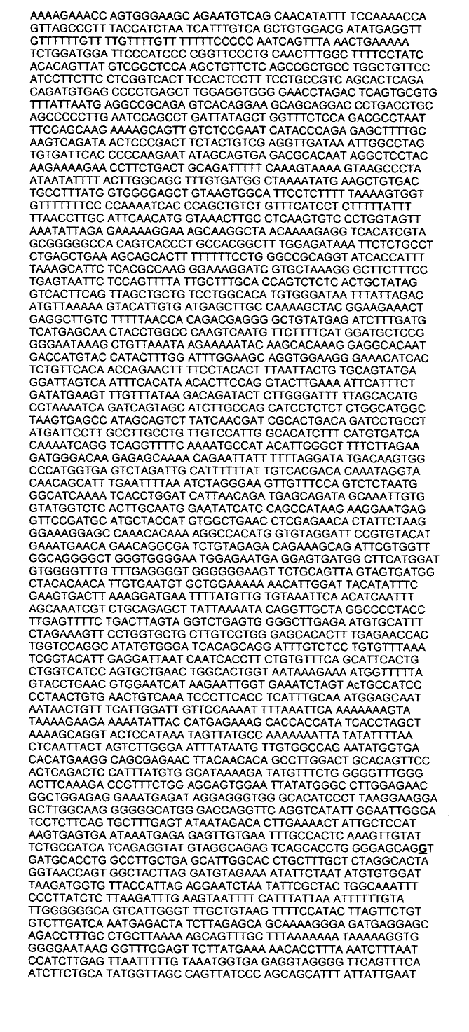

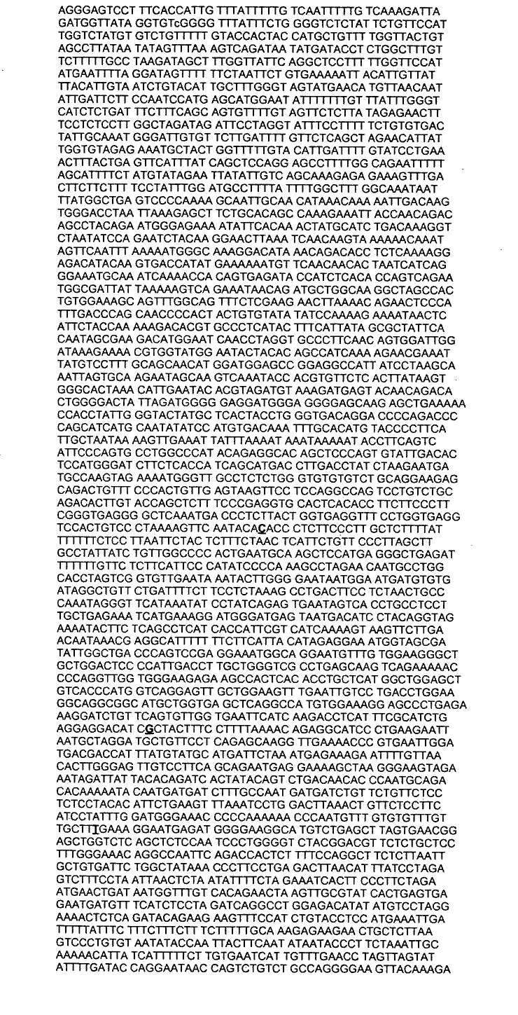

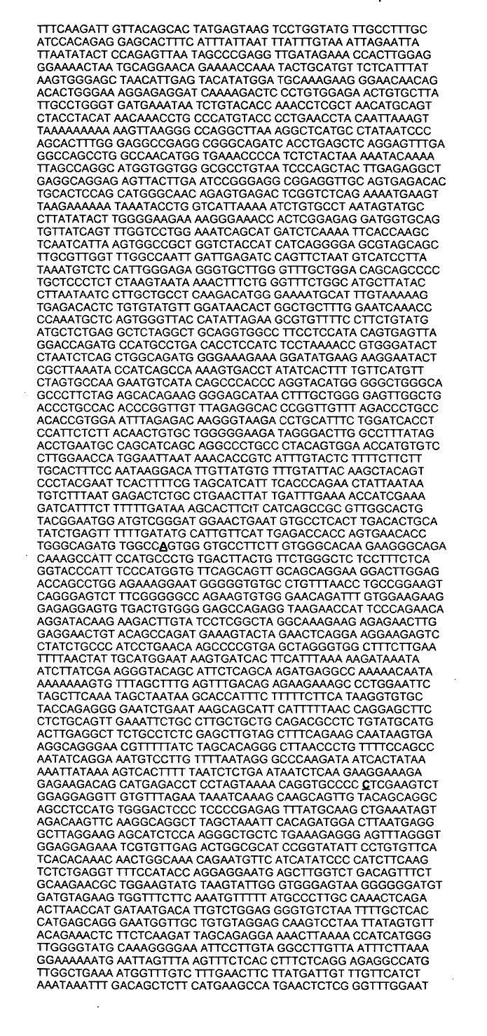

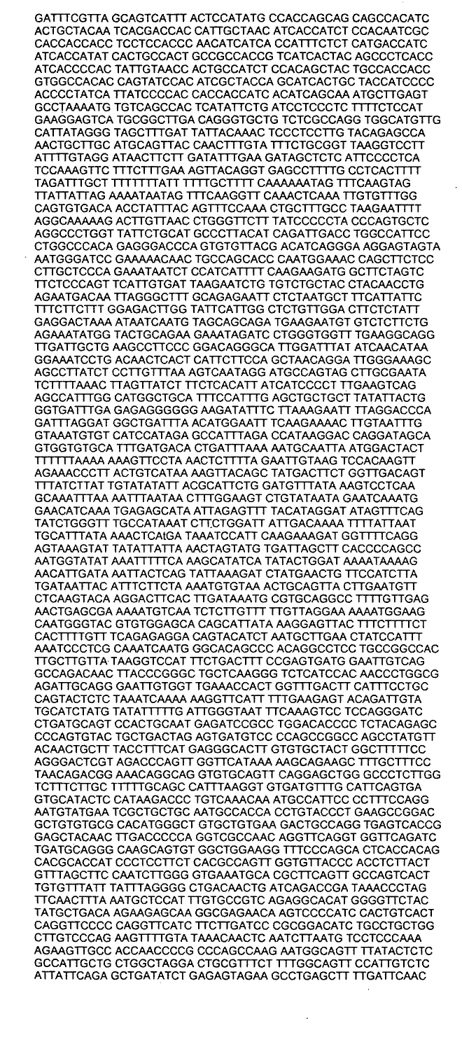

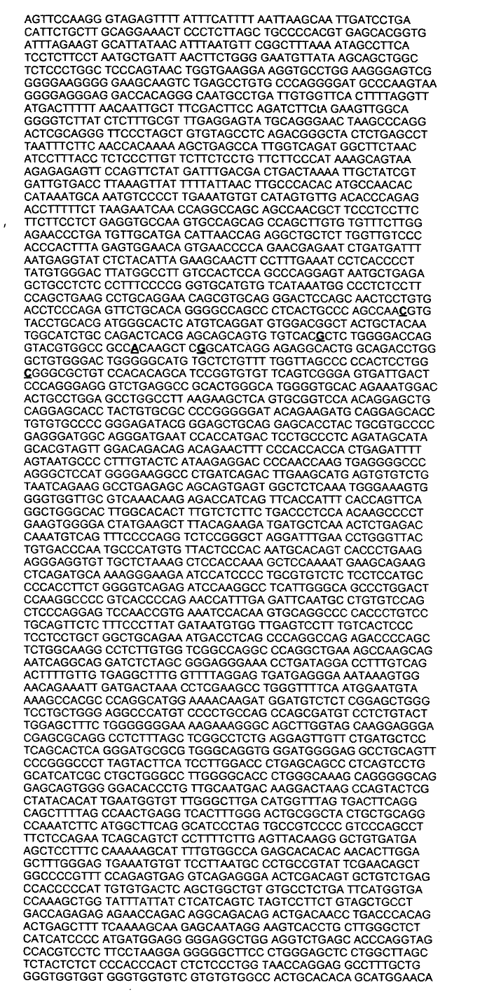

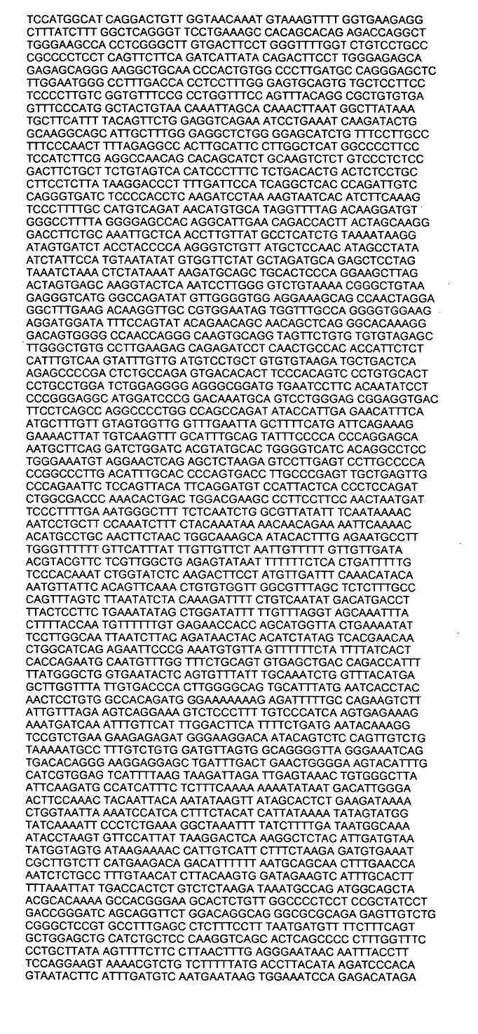

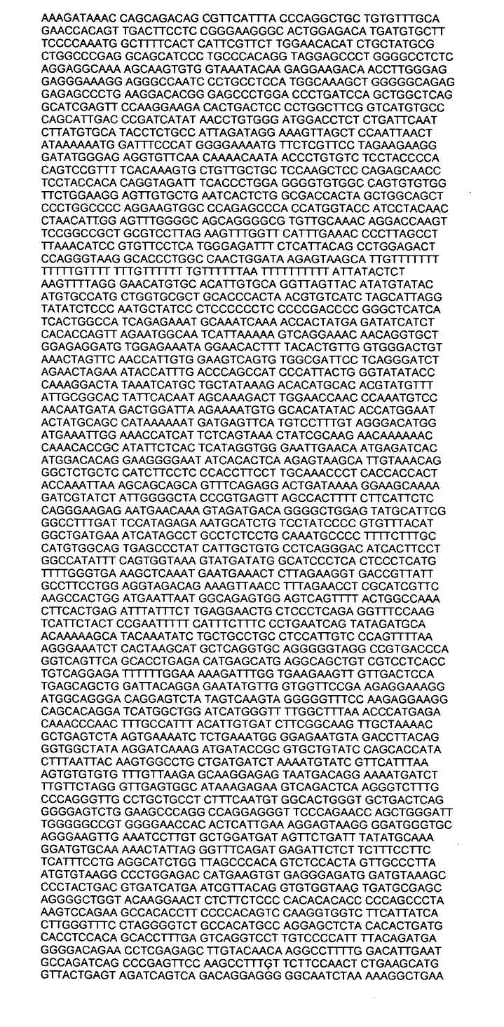

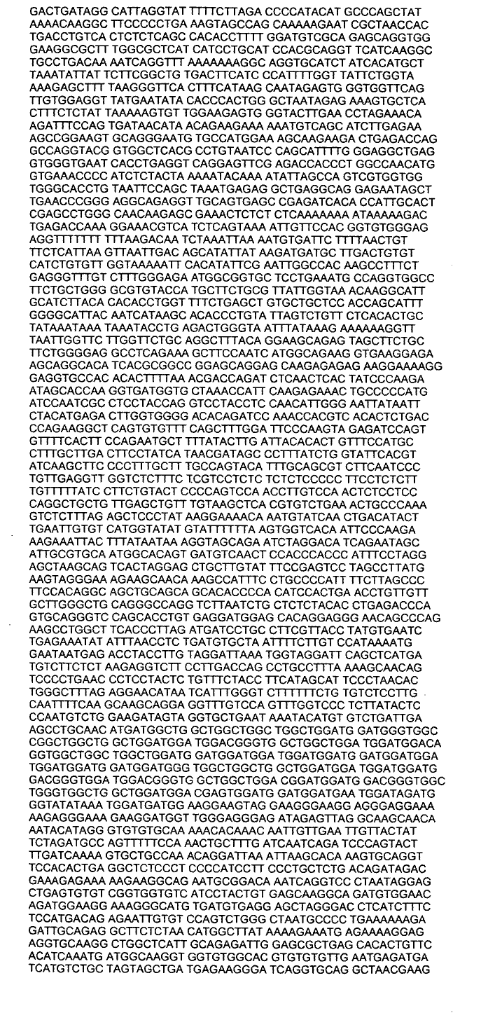

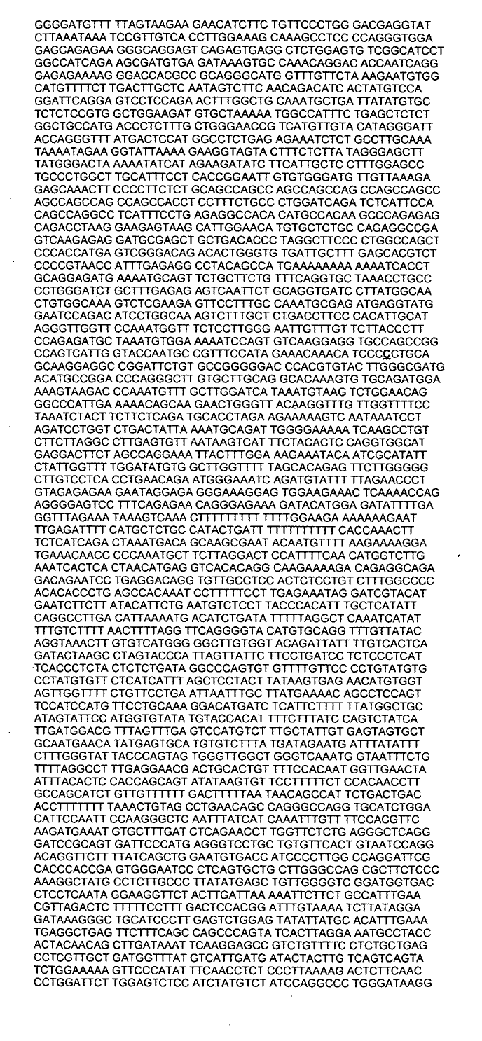

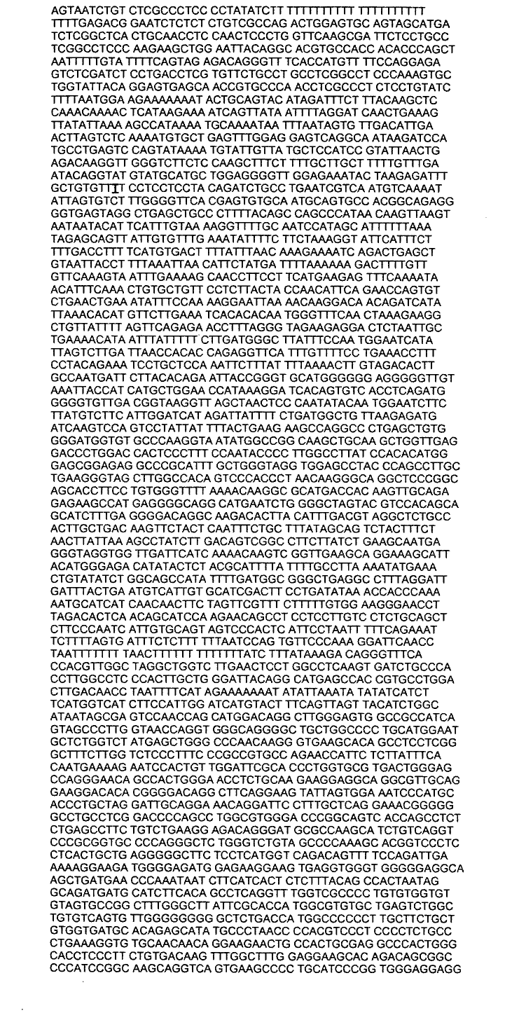

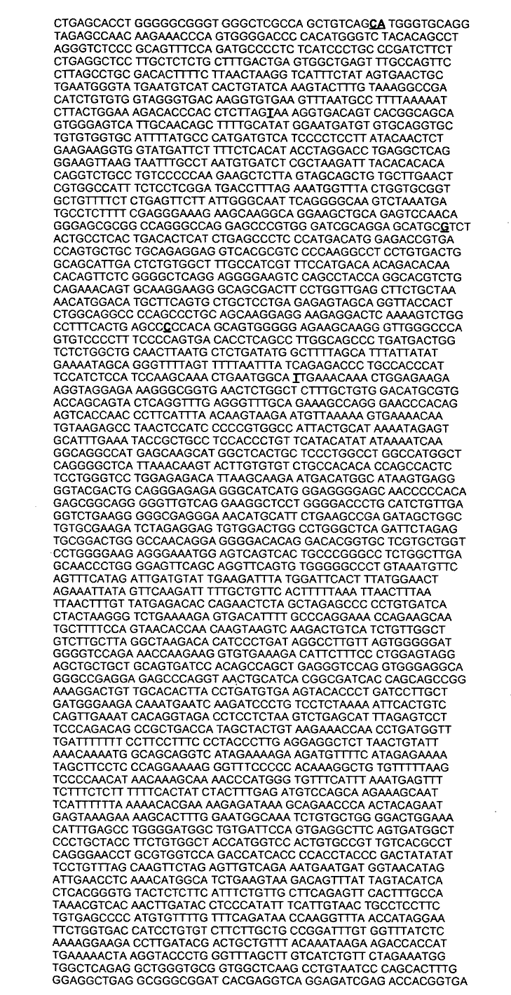

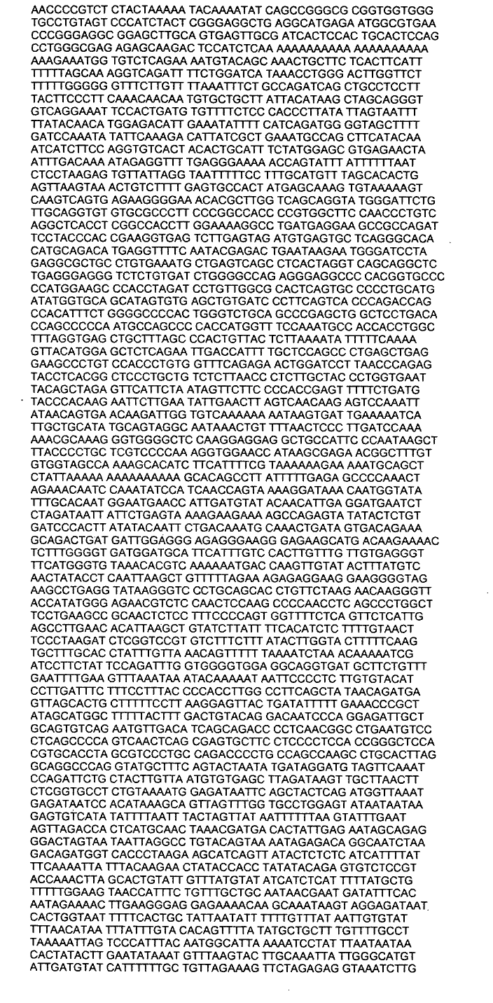

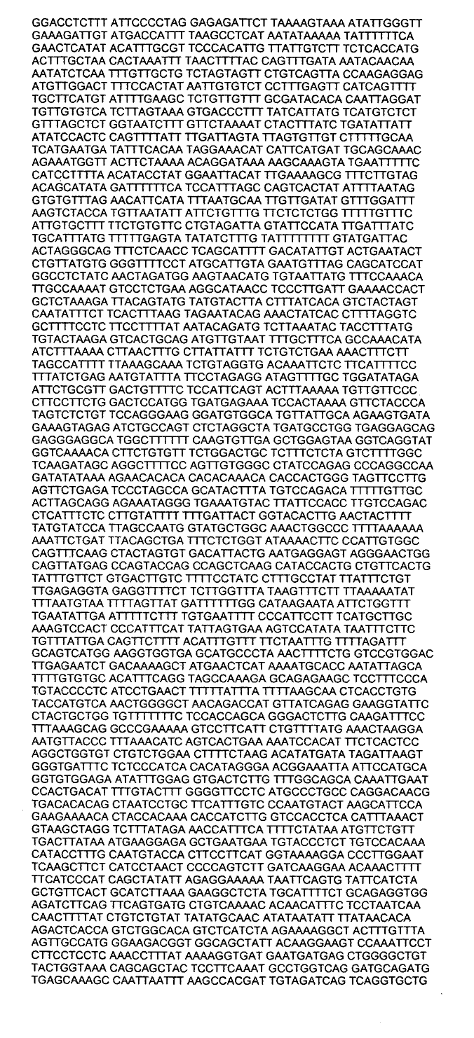

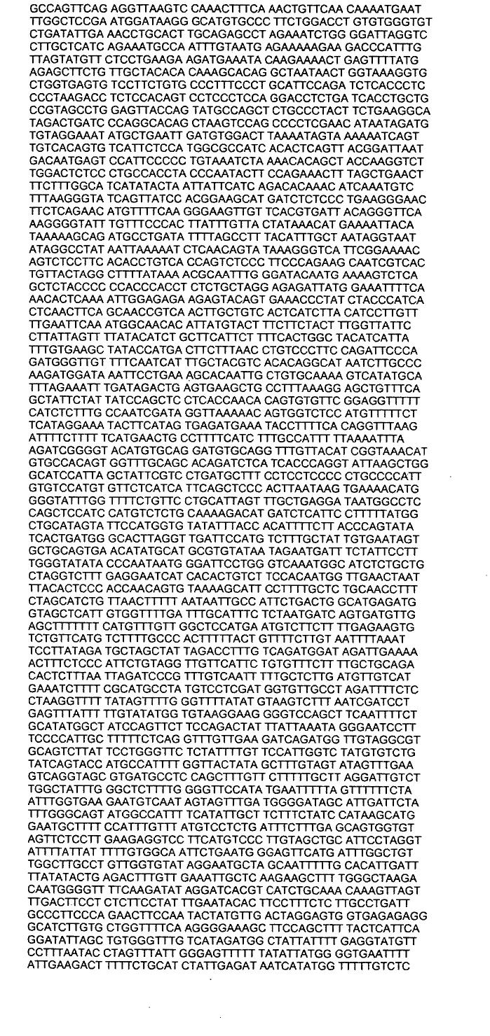

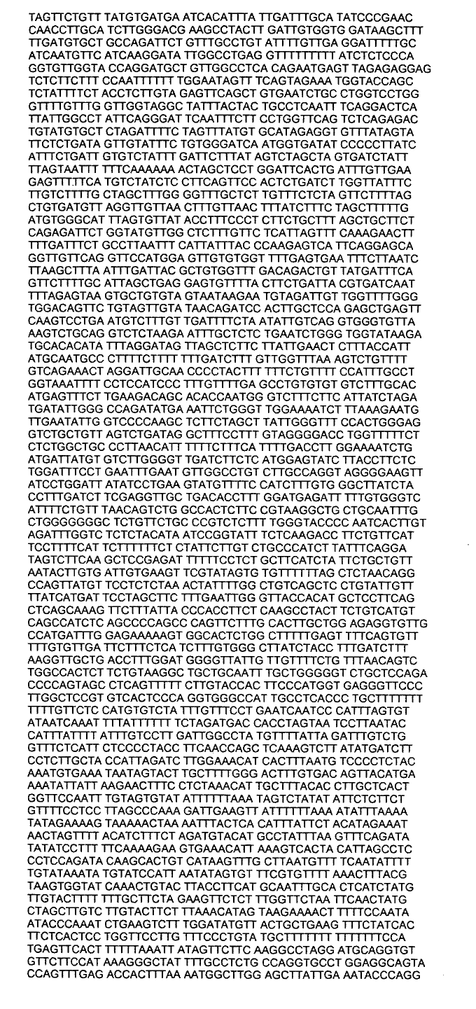

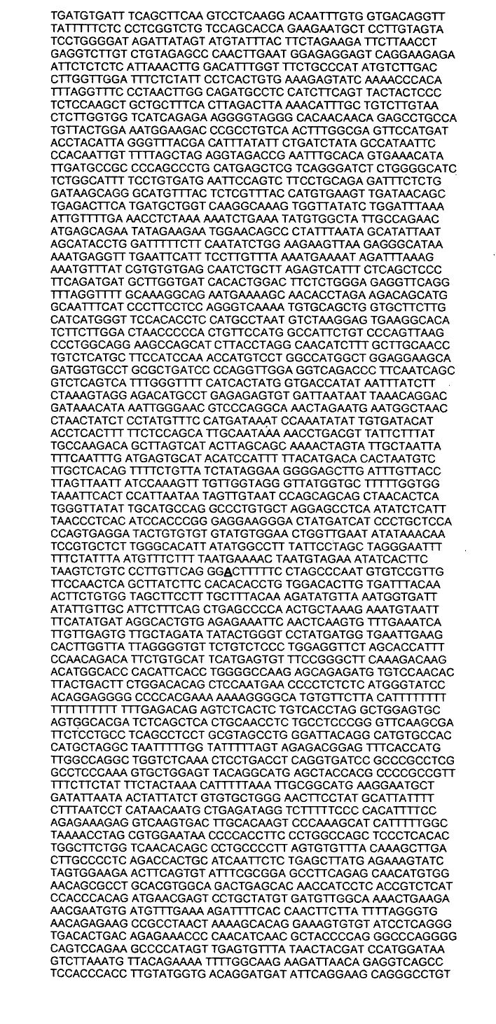

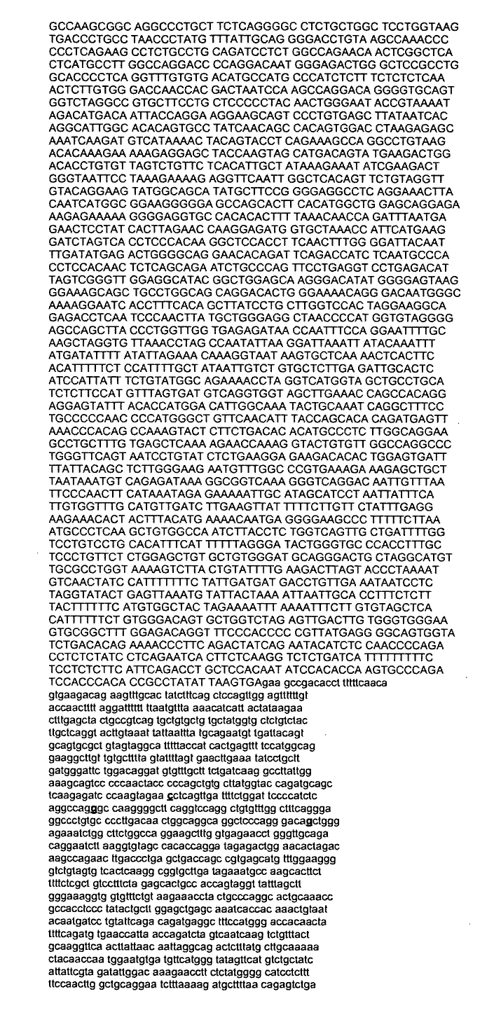

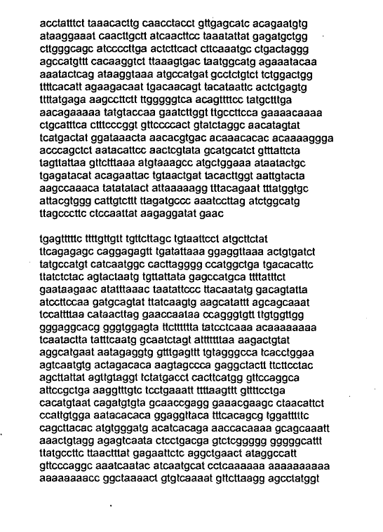

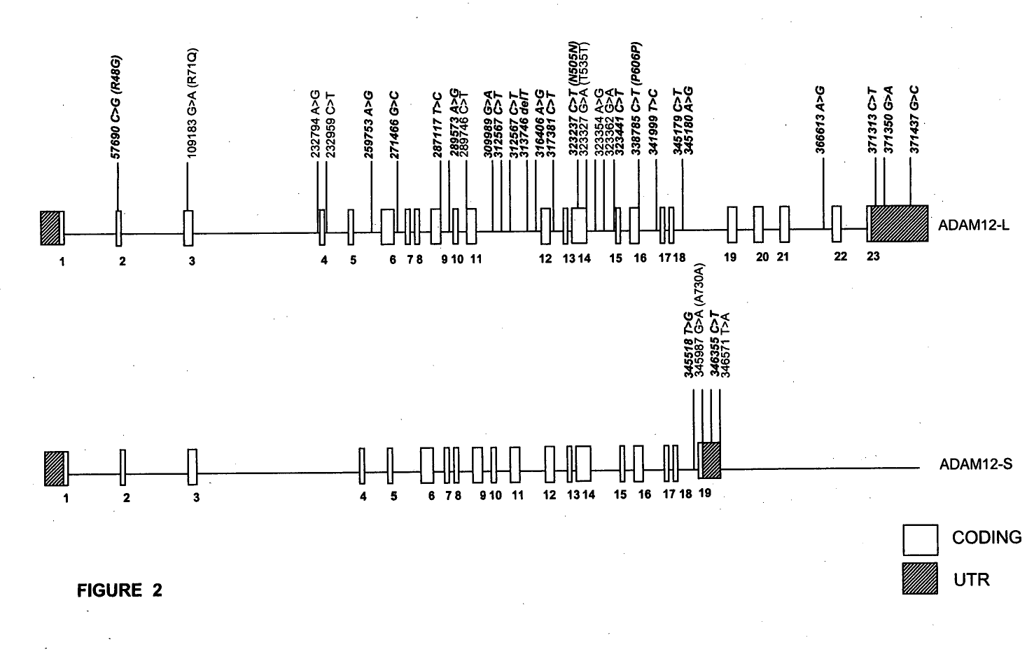

FIG. 2

Schematic of the ADAM12 gene displaying the positions of the single nucleotide polymorphisms in relation to the exons, introns and untranslated regions (UTRs). SNPs demonstrating significant association (p<0.1) are displayed in bold.

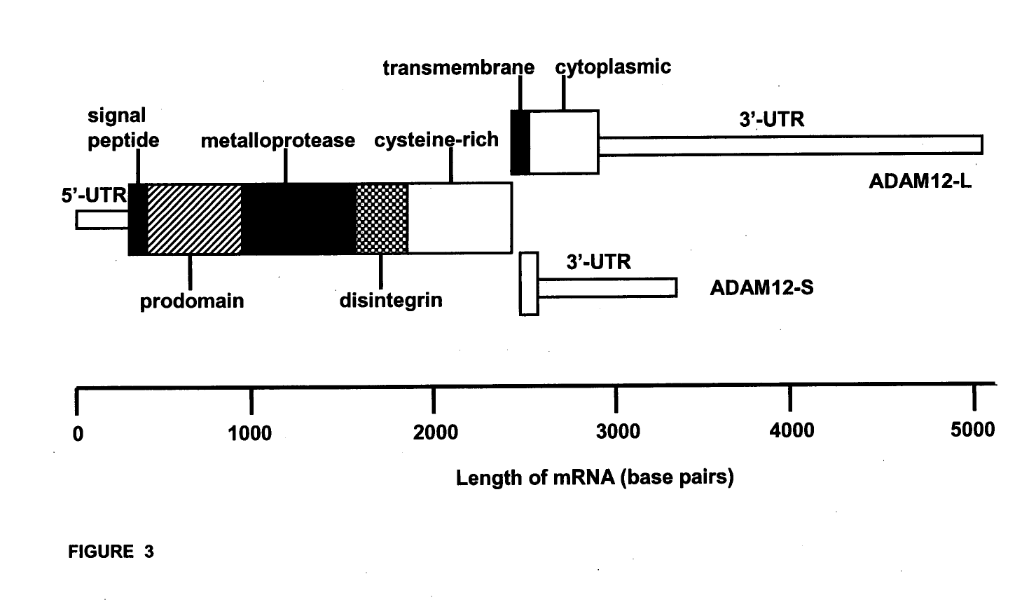

FIG. 3

Schematic of the ADAM12 protein isoforms ADAM1 2-S and ADAM12-L, showing the location of functional domains.

Table 1A

DNA sequence of SNPs in the ADAM12 gene and the nucleic acids located 50 bp upstream of each SNP and 50 bp downstream of each SNP.

Table 1B

Table 1B shows the location of the SNP in the gene and the effect of each SNP within the ADAM12 genomic DNA sequence.

Table 1C

DNA sequences of oligonucleotide primers used to amplify regions of the ADAM12 gene containing SNPs identified herein.

Table 2A

Linkage disequilibrium results for 8 SNPs from the ADAM12 gene.

Table 2B

Haplotype association data for SNPs associated with ADAM12 mediated disease and, in particular, Alzheimer's Disease.

Table 2C

Shows the statistical significance of the SNPs and their ability to act as markers or indicators of ADAM12 mediated disease and in particular Alzheimer's Disease.

Materials and Methods

ADAM12 encodes two splice variants; ADAM12-S, a shorter secreted form and ADAM12-L, a longer membrane-bound form, that diverge at their 3′ ends (FIG. 2). The mRNA sequence for each splice variant (ADAM12-L: NM_{—}003474; ADAM12-S: NM_{—}021641) was obtained from the GenBank database at NCBI (http://www.ncbi.nlm.nih.gov). Determination of coding sequences, untranslated regions (UTRS) and intronic regions was based on alignment of the mRNA sequences with genomic clone sequences, using BLAST sequence homology searches (http://www.ncbi.nlm.nih.gov/blast/Blast.cgi). The genomic clones identified were AC063963, AC022015, AL691463, AC026226 and AL589787. This genomic sequence was assembled into a contig using the Sequencher™ program. ADAM12-L spans 373,186 bp and ADAM12-S spans 347,114 bp.

Case-control Pools

Five DNA pools were analysed for each SNP; the first pair of pools consisted of DNA from 96 UK Caucasian AD patients and 96 age- and sex-matched controls respectively. These will be referred to as the Cohort 1 case pool and the Cohort 1 control pool. The second pair of Cohort pools (Cohort 2) consisted of DNA from 90 UK Caucasian AD patients and 90 age- and sex-matched controls respectively. The overall average age at onset of the AD patients in the two case pools was 74.6 and 73.9 years; the overall age at collection of the controls in the two control pools was 76 and 74.8 years. Cohort 3 consisted of DNA from 57 sib-pairs, concordant for LOAD, with an average age at onset of 77 years. All patients were diagnosed according to NINCDS-ADRDA criteria (McKhann et al., 1984). Cognitive function of controls was assessed using the mini mental-state exam (MMSE) (Folstein et al., 1975) and only those with a score of at least 28 were included in the study.

The mutation screening sample consisted of 14 UK Caucasian AD patients, with age at onset >65 years. The sample size of 14 subjects screened across all exons gives power of 80% to detect alleles with a frequency of 0.05 or above. This estimate ignores the fact that our sample is enriched for AD susceptibility alleles, and the true power is correspondingly (but unquantifiably) greater. High molecular weight genomic DNA was extracted from whole blood or transformed lymphoblasts following standard laboratory procedures.

Mutation Detection

ADAM12 encodes two splice variants; ADAM12-S, a shorter secreted form and ADAM12-L, a longer membrane-bound form, that diverge at their 3′ ends (FIG. 1). The mRNA sequence for each splice variant was obtained from the GenBank database at NCBI (http://www.ncbi.nlm.nih.gov). Determination of coding sequences, untranslated regions (UTRs) and intronic regions was based on alignment of the mRNA sequences with genomic clone sequences, using BLAST sequence homology searches (http://www.ncbi.nlm.nih.gov/blast/Blast.cgi).

PCR fragments spanning exons, UTRs and limited 5′ flanking regions were designed using Primer 3.0 (http://www-genome.wi.mit.edu/cgi-bin/primer/primer3_www.cgi). PCR amplification was performed under standard conditions of 1× PCR buffer (Qiagen), 1.5 mM MgCl₂, 250 μM dNTPs, 0.5 μM of each primer, 0.6 units Hot Start Taq (Qiagen) and 36 ng genomic DNA in a 24 μl reaction. Cycling was conducted in a MJ Tetrad (MJ Research) with an initial denaturation of 94° C. for 15 min, followed by 35 cycles of 94° C. for 30 s, appropriate annealing temperature for 30 s and 72° C. for 45 s with a final extension step of 72° C. for 10 min. Synthesis of appropriately sized PCR products was confirmed by electrophoresis on 2% agarose gels.

Polymorphisms were identified by DHPLC using a Wave™ DNA Fragment Analysis System (Transgenomic) as previously described (Abraham et al., 2001). The 14 screening samples were amplified as described above, except the final extension in the PCR protocol was followed by denaturation at 94° C. for 5 min and then cooling to 65° C. over 30 min, to allow heteroduplex formation. Column temperature and acetonitrile gradient were determined using the DHPLC Melt program (httl://insertion.stanford.edu/melt1.html). To ensure maximum sensitivity, in addition to the temperature suggested by the software (n° C.), each fragment was also run at n+2° C. Samples showing heteroduplex formation were sequenced to identify the variant.

PCR products were purified through QlAquick columns (Qiagen) to remove unincorporated primers and dNTPs. Purified products were then bidirectionally sequenced on an ABI 3100 Genetic Analyser (Applied Biosystems) using the Big Dye Terminator (v2.0) Cycle Sequencing kit (Applied Biosystems). Sequence traces were subsequently exported to Sequencher™ (Applied Biosystems) to characterise polymorphisms.

Genotyping

Each SNP was typed in pools by primer extension, using the ABI SNaPshot™ Multiplex kit. Extension primers were designed to be 15-30 nucleotides long and directly adjacent to the polymorphism. For each SNP, every pool was PCR amplified in duplicate. An individual DNA sample, heterozygous for each SNP was also amplified. Samples were then purified by incubation with 1 unit each of exonuclease I and shrimp alkaline phosphatase at 37° C. for 1 hour. Primer extension was then performed according to SNaPshot™ kit instructions, and products electrophoresed on the ABI 3100 Genetic Analyser. The resultant data was analysed using Genotyper® 2.5.

Statistical Analysis

Statistical significance of difference between allele distributions was assessed using a Monte-Carlo method as implemented in the CLUMP program. (Sham P C, Curtis D, (1995) Monte Carlo tests for associations between disease and alleles at highly polymorphic loci. Ann Hum Genet, 59, 97-105.) This approach determines an empirical P value by estimating by simulation the proportion of times the observed χ² for the contingency table might be expected to occur by chance, conditional on the marginal totals of the table.

Results

SNPs Identified

A total of 32 SNPs were identified in ADAM12 (FIG. 2 and Table 1B). Two of these SNPs were non-synonymous coding SNPs; an R48G polymorphism in exon 2 and an R71Q polymorphism in exon 3. Both of these polymorphisms are located in the prodomain of the latent protein. It is possible that either amino acid change could affect the proper folding of the protein. This could result in its retention in the endoplasmic reticulum and subsequent degradation. Four synonymous coding SNPs were also detected: an N505N SNP in exon 14; a T535T SNP in exon 14; a P606P SNP in exon 16; and an A730A in exon 19 of the ADAM12-S variant. Two SNPs were identified in the 3′UTR of the ADAM12-S variant, three SNPs were identified in the 3′UTR of the ADAM12-L variant and the remaining SNPs were intronic (FIG. 2 and Table 1B).

LD SNP Association and Haplotype Association Analysis

All polymorphisms identified were tested for association with LOAD in Cohorts 1, 2 and 3. These results are displayed in Table 2A. Moreover, those haplotypes that were significant indicators of AD are shown in Table 2B.

TABLE 1A

| SNP No | SNP ID | 50 bp 5′ TO SNP | SNP | 50 bp 3′ TO SNP |
|---|---|---|---|---|
| 1 | g57690 C > G | TGAGCTTATGGAACCAAGGAAGAGCTGATGAAGTTGTCAGTGCCTCTG [symbol] | C/G | GGAGTTGGGGACCTCTGGATCCCAGTGAAGAGCTTCGACTCCAAGGTGA [symbol] |
| 2 | g109183 G > A | CTGACTTTTGTTGTTTATCTTTCAGAATCATCCAGAAGTGCTGAATATT [symbol] | G/A | ACTACAACGGGAAAGCAAAGAACTGATCATAAATCTGGAAAGAAATGAGT [symbol] |
| 3 | g232794 A > G | AGGTCACAATCTTGCTACCCTATAGCCTAGACATTCACAACAATGTAT [symbol] | A/G | TGCAATTCTGCACTCATCTTTTTATGGTGTTTTTGTTTTTCCGCAGAGGT |
| 4 | g232959 C > T | GAAATTACACGGTAATTCTGGCACGTTGGGATCACCATAATCTTACAC [symbol] | C/T | TGGATCTGTGCTTAACCTTTGTAGCCATTTTATTCGGAGCAAAATCACAT |
| 5 | g259753 A > G | CATGAGAATGGGGCAGATGTTATTGTCACCTGAGCAGCCAACTAAGG [symbol] | A/G | GCATCCTCAGGGGCCTGGAGTTCGTTTCCCAACCCCCAGCATTGCTGTC [symbol] |
| 6 | g271466 G > C | TTCTACTTGTGTAACCCTCACGTCTTCTGTTTGCTAGTATGTTTCTAAGA [symbol] | G/C | TTTCTATGGCAGGTGCTCACAACCTCTTTTTCGTTTCCTGCCCCATCACA |
| 7 | g287117 T > C | CTTGTCAGGTAGGGAGTTACGTCTTGTCTGCTTAAAAAAAAAAGGAGT [symbol] | T/C | CTCGGCGAAGCCCTTCGTTTCACCTGCACAGCCCCAGGAGCTGCCACCT [symbol] |
| 8 | g289573 A > G | CCCTCATACAGCTATTTTTATTTTTGCGTATTTTATTGCAGGAATATCCA [symbol] | A/G | CAGAATTTTTAAATATTAGAGAATGCCCCCGTTCTAATTTGATTAAAGAA |
| 9 | g289746 C > T | TGTGCACGGCAGACCAGTCTGGGGGAATTGTCATGGTAAGCCAAGGG [symbol] | C/T | GGGAGCTGGTTCCCCTTGATAATGATTCCCCAGGAAGTCACTACTTTTAT [symbol] |
| 10 | g309989 G > A | ATTCTGCCATCATCAGAGGTATGTAGGCAGAGTCAGCACCTGGGGAGCAG | A/G | TGATGCACCTGGCCTTGCTGAGCATTGGCACCTGCTTTGCTCTAGGCACT [symbol] |
| 11 | g312567 C > T | TACTGGTGAGGTTTCCTGGTGAGGTCCACTGTCCCTAAAAGTTCAATACA | C/T | ACCCTCTTCCCTTGCTCTTTTATTTTTTTCTCCTTAATTCTACTCTTTCT |
| 12 | g313352 G > A | CAGTGTTGGTGAATTCATCAAGACCTCATTTCGCATCTGAGGAGGACA [symbol] | A/G | CTACTTTCCTTTTAAAACAGAGGCATCCCTGAAGAATTAATGCTAGGATG |
| 13 | g313746 delT | ATTTGGATGGGAAACCCCCAAAAAACCCAATGTTTGTGTGTTTGTTGCT [symbol] | T/− | GAAAGGAATGAGATGGGGAAGGCATGTCTGAGCTTAGTGAACGGAGCT [symbol] |
| 14 | g316406 A > G | ATATGCATTGTTCATTGAGACCACCAGTGAACACCTGGGCAGATGTGG [symbol] | A/G | GTGGGTGCCTTCTTGTGGGCACAAGAAGGGCAGACAAAGCCATTCCATG [symbol] |
| 15 | g317381 C > T | GAAGGAAAGAGAGAAGACAGCATGAGACCTCCTAGTAAAACAGGTGC [symbol] | C/T | TCGAAGTCTGGAGGAGGTTGTGTTTAGAATAAATCAAAGCAAGCAGTTGT [symbol] |
| 16 | g323237 C > T | TGTGACCTCCCAGAGTTCTGCACAGGGGCCAGCCCTCACTGCCCAGC [symbol] | C/T | GTGTACCTGCACGATGGGCACTCATGTCAGGATGTGGACGGCTACTGCT [symbol] |
| 17 | g323327 G > A | GGCTACTGCTACAATGGCATCTGCCAGACTCACGAGCAGCAGTGTGT [symbol] | G/A | CTCTGGGGACCAGGTACGTGGCCGCCRCAAGCTCRGCATCAGGAGAGG [symbol] |
| 18 | g323354 A > G | ACTCACGAGCAGCAGTGTGTCACRCTCTGGGGACCAGGTACGTGGCC [symbol] | A/G | CAAGCTCRGCATCAGGAGAGGCACTGGCAGACCTGGGCTGTGGGACTG [symbol] |
| 19 | g323362 G > A | GCAGCAGTGTGTCACRCTCTGGGGACCAGGTACGTGGCCGCCRCAA [symbol] | G/A | GCATCAGGAGAGGCACTGGCAGACCTGGGCTGTGGGACTGGGGGCATG [symbol] |
| 20 | g323441 C > T | GCTGTGGGACTGGGGGCATGTGCTCTGTTTTGGTTAGCCCCCACTCC [symbol] | C/T | GGGCGCTGTCCACACAGCATCCGGTGTGTTCAGTCGGGAGTGATTGACT [symbol] |
| 21 | g338785 C > T | AGCCGGCCAGTCATTGGTACCAATGCCGTTTCCATAGAAACAAACATC [symbol] | C/T | CTGCAGCAAGGAGGCCGGATTCTGTGCCGGGGGACCCACGTGTACTTG [symbol] |
| 22 | g341999 T > C | ATGTATGCATGCTGGAGGGGTTGGAGAAATACTAAGAGATTTGCTGTG [symbol] | T/C | TCCTCCTCCTACAGATCTGCCTGAATCGTCAATGTCAAAATATTAGTGTC |
| 23 | g345179 C > T | GGTGGGAGGAGGCTGAGCACCTGGGGGCGGGTGGGCTCGCCAGCT [symbol] | C/T | RTGGGTGCAGGTAGAGCCAACAAGAAACCCAGTGGGGACCCCACATGG [symbol] |
| 24 | g345180 A > G | GTGGGAGGAGGCTGAGCACCTGGGGGCGGGTGGGCTCGCCAGCTGT [symbol] | A/G | TGGGTGCAGGTAGAGCCAACAAGAAACCCAGTGGGGACCCCACATGGG [symbol] |
| 25 | g345518 T > G | GAAGTTTAATGCCTTTTAAAAATCTTACTGGAAAGACACCACCTCTTA [symbol] | T/G | AAAGGTGACAGTCACGGCAGCAGTGGGAGTCATTGCAACAGCTTTTGCA [symbol] |
| 26 | g345987 G > A | AACAGGGAGCGCGGCCAGGGCCAGGAGCCCGTGGGATCGCAGGAG [symbol] | G/A | TCTACTGCCTCACTGACACTCATCTGAGCCCTCCCATGACATGGAGACC [symbol] |
| 27 | g346355 C > T | CCCTGCAGCAAGGAGGAAGAGGACTCAAAAGTCTGGCCTTTCACTGA [symbol] | C/T | CCACAGCAGTGGGGGAGAAGCAAGGGTTGGGCCCAGTGTCCCCTTTCC [symbol] |
| 28 | 9346571 T > A | TCAGAGACCCTGCCACCCATTCCATCTCCATCCAAGCAAACTGAATGG [symbol] | T/A | TGAAACAAACTGGAGAAGAAGGTAGGAGAAAGGGCGGTGAACTCTGGCT [symbol] |
| 29 | g366613 A > G | ATGAAAACTAATGTAGAAATATCACTTCTAAGTCTGTCCCTTGTTCAGG [symbol] | A/G | CTTTTTCCTAGCCCAATGTGTCCGTTGTTCCAACTCAGCTTATCTTCCAC |
| 30 | g371311 C > T | CCCAGCTGTGCTTATGGTACCAGATGCAGCTCAAGAGATCCCAAGTAG [symbol] | C/T | CTCAGTTGATTTTCTGGATTCCCCATCTCAGGCCAGRGCCAAGGGGCTT [symbol] |
| 31 | g371348 G > A | ATCCCAAGTAGAAYCTCAGTTGATTTTCTGGATTCCCCATCTCAGGCCA [symbol] | G/A | GCCAAGGGGCTTCAGGTCCAGGCTGTGTTTGGCTTTCAGGGAGGCCCT [symbol] |
| 32 | g371435 G > C | CAGGGAGGCCCTGTGCCCCTTGACAACTGGCAGGCAGGCTCCCAGG [symbol] | G/C | CTGGGAGAAATCTGGCTTCTGGCCAGGAAGCTTTGGTGAGAACCTGGGT [symbol] |

TABLE 1B

| SNP No | SNP ID | AA Change | Location in Gene | rs # |
|---|---|---|---|---|
| 1 | g57690 C > G | R48G | Exon 2 Arg/Gly | rs3740199 |
| 2 | g109183 G > A | R71Q | Exon 3 Arg/Gln | |
| 3 | g232794 A > G | | 47 bp upstream exon 4 | rs1466361 |
| 4 | g232959 C > T | | 31 bp downstream exon 4 | rs1466360 |
| 5 | g259753 A > G | | 7191 bp downstream exon 5 | rs1278319 |
| 6 | g271466 G > C | | 1358 bp downstream exon 6 | rs1278390 |
| 7 | g287117 T > C | | 43 bp downstream exon 9 | rs2290845 |
| 8 | g289573 A > G | | 73 bp upstream exon 10 | rs2290844 |
| 9 | g289746 C > T | | 16 bp downstream exon 10 | rs2290842 |
| 10 | g309989 G > A | | 6503 bp upstream exon 12 | |
| 11 | g312567 C > T | | 3925 bp upstream exon 12 | rs3781013 |
| 12 | g313352 G > A | | 3140 bp upstream exon 12 | rs12415953 |
| 13 | g313746 delT | | 2746 bp upstream exon 12 | |
| 14 | g316406 A > G | | 86 bp upstream exon 12 | rs2290841 |
| 15 | g317381 C > T | | 711 bp downstream exon 12 | rs1152653 |
| 16 | g323237 C > T | N505N | Exon 14 Asn/Asn | rs1278279 |
| 17 | g323327 G > A | T535T | Exon 14 Thr/Thr | rs2279091 |
| 18 | g323354 A > G | | 14 bp downstream exon 14 | rs2279090 |
| 19 | g323362 G > A | | 22 bp downstream exon 14 | |
| 20 | g323441 C > T | | 101 bp downstream exon 14 | rs1278278 |
| 21 | g338785 C > T | P606P | Exon 16 Pro/Pro | rs2292692 |
| 22 | g341999 T > C | | 15 bp upstream exon 17 | rs11244787 |
| 23 | g345179 C > T | | 73 bp downstream exon 18 | rs7922601 |
| 24 | g345180 A > G | | 74 bp downstream exon 18 | rs7911793 |
| 25 | g345518 T > G | | 393 bp upstream exon 19 S | rs1278260 |
| 26 | g345987 G > A | A730A | Exon 19 S Ala/Ala | |
| 27 | g346355 C > T | | 3′UTR S | rs6693 |
| 28 | g346571 T > A | | 3′UTR S | rs4732 |
| 29 | g366613 A > G | | 1708 bp upstream exon 22 L | rs872328 |
| 30 | g371311 C > T | | 3′UTRL | rs7913591 |
| 31 | g371348 G > A | | 3′UTRL | rs7916918 |
| 32 | g371435 G > C | | 3′UTRL | rs10751538 |

TABLE 1C

| | | | |
|---|---|---|---|
| SNP | | | |
| No | SNP ID | PCR F PRIMER | PCR R PRIMER |

| | | | |
|---|---|---|---|
| 1 | g57690 C > G | GCTGATGCCAAACTCTTCCT | CTTCGGCAGTCTCAAAGCTG |
| 2 | g109183 G > A | TCATTTGACTACGCCTGTGG | GGTGTCTTAACTTTCTCCTATCATGC |
| 3 | g232794 A > G | TTGCTCCGAATAAAATGGCTAC | CTAAACTGGGAAGGCCCTAATG |
| 4 | g232959 C > T | TTGCTCCGAATAAAATGGCTAC | CTAAACTGGGAAGGCCCTAATG |
| 5 | g259753 A > G | CACCTGAGCAGCCAACTAAG | AAGGTGAGGTGCCAATTCAG |
| 6 | g271466 G > C | AAACTGCTCTTATCGGAACCAG | CATGTCAAGGTGAGCCACAG |
| 7 | g287117 T > C | TACCTCGCAAATCCCATGAC | CAGGGTGTTCAGAGGGAGAC |
| 8 | g289573 A > G | TTATCAAGGGGAACCAGCTC | ACAGAACCATGACGCAACTG |
| 9 | g289748 C > T | TTATCAAGGGGAACCAGCTC | ACAGAACCATGACGCAACTG |
| 10 | g309989 G > A | | |
| 11 | g312567 C > T | | |
| 12 | g313352 G > A | | |
| 13 | g313746 delT | | |
| 14 | g316408 A > G | TGGAACTGAATGTGCCTCAC | TGGTCTCCAAGTCCTTCCTG |
| 15 | g317381 C > T | TGTTTTCCAGCCAATATCAGG | TAAACTCTCGGGGAGGGAGT |
| 16 | g323237 C > T | GTCTGCCAGTGCCTCTCC | GGGTGCATGTGTCATAAATGG |
| 17 | g323327 G > A | GTCTGCCAGTGCCTCTCC | GGGTGCATGTGTCATAAATGG |
| 18 | g323354 A > G | GTCTGCCAGTGCCTCTCC | GGGTGCATGTGTCATAAATGG |
| 19 | g323362 G > A | GTCTGCCAGTGCCTCTCC | GGGTGCATGTGTCATAAATGG |
| 20 | g323441 C > T | CTCACGAGCAGCAGTGTGTC | CTCCAGGCAGTGTCCATTTC |
| 21 | g338785 C > T | AACCTTGTAACCCAGTTCTTGC | GTTCCAAATGGTTTCTCCTTGG |
| 22 | g341999 T > C | GCTGTAAAAGGGCAGCTCAG | AGGTTGGGTCTTCTCCAAGC |
| 23 | g345179 C > T | GGTTTCTTGTTGGCTCTACCTG | ATATGCCCTAACCCCACGTC |
| 24 | g345180 A > G | GGTTTCTTGTTGGCTCTACCTG | ATATGCCCTAACCCCACGTC |
| 25 | g345518 T > G | AGTTCCTTAGCCTGCGACAC | GCAATGACTGCCACTGCTG |
| 26 | g345987 G > A | CTTGCTTCTCCCCCACTG | CAGGGGCAAGTCTAAATGATG |
| 27 | g346355 C > T | TGTTTAATGAGCCCCTGAGC | GCAGCAAGGAGGAAGAGGAC |
| 28 | g346571 T > A | TGTTTAATGAGCCCCTGAGC | GCAGCAAGGAGGAAGAGGAC |
| 29 | g366813 A > G | CTGCTCCACCAGTGAGGATAC CAGGTGTGTGGAAGATAAGCTG | |
| 30 | g371311 C > T | TACATCTCCAACCCCAGACC | CCTGAAAGCCAAACACAGC |
| 31 | g371348 G > A | TACATCTCCAACCCCAGACC | CCTGAAAGCCAAACACAGC |
| 32 | g371435 G > C | CTGGATTCCCCATCTCAGG | ACCCATGAACATGACATTCC |

TABLE 2A

| | 3) | 6) | 8) | 12) | 13) | 19) | 20) | 21) |
|---|---|---|---|---|---|---|---|---|
| | 109183 | 289575 | 323239 | 338787 | 342001 | 371313 | 371350 | 371437 |
| SNP r² | G > A | A > G | C > T | C > T | T > C | C > T | G > A | G > C |
| 1) 57690 C > G | 0.002 | 0.030 | 0.000 | 0.004 | 0.005 | 0.038 | 0.038 | 0.026 |
| 2) 109183 G > A | | 0.003 | 0.026 | 0.027 | 0.061 | 0.023 | 0.034 | 0.021 |
| 8) 289573 A > G | | | 0.226 | 0.028 | 0.036 | 0.000 | 0.000 | 0.002 |
| 16)23237 C > T | | | | 0.672 | 0.630 | 0.177 | 0.144 | 0.116 |
| 21)338785 C > T | | | | | 1.000 | 0.060 | 0.039 | 0.017 |
| 22)341999 T > C | | | | | | 0.052 | 0.052 | 0.007 |
| 30) 371311 C > T | | | | | | | 1.000 | 0.926 |
| 31) 371348 G > A | | | | | | | | 0.925 |

Linkage Disequilibrium (LD) Results for 8 SNPs genotyped in 90 individuals. r² values are displayed. Highly significant values are shown in bold.
SNPs 21 (338785 C > T) and 13 (341999 T > C) are in total LD
SNPs 30 (371311 C > T) and 20 (371348 G > A) are also in total LD
SNPs 30/31 and 21 (371435 G > C) are in very high, but not total LD

TABLE 2B

| 3-Marker Haplotype | p-value | 3-Marker Haplotype | p-value | 3-Marker Haplotype | p-value | 3-Marker Haplotype | p-value |
|---|---|---|---|---|---|---|---|
| 1_2_8 | 0.0126 | 2_8_16 | 0.0452 | 8_16_22 | 0.0598 | 16_22_31 | 0.0223 |
| 1_2_16 | 0.3048 | 2_8_22 | 0.0205 | 8_16_31 | 0.0192 | | |
| 1_2_22 | 0.1134 | 2_8_31 | 0.0066 | 8_22_31 | 0.0287 | | |
| 1_2_31 | 0.0160 | 2_16_22 | 0.1256 | | | | |
| 1_8_16 | 0.0127 | 2_16_31 | 0.0130 | | | | |
| 1_8_22 | 0.0074 | 2_22_31 | 0.0406 | | | | |
| 1_8_31 | 0.0021 | | | | | | |
| 1_16_22 | 0.0473 | | | | | | |
| 1_16_31 | 0.0046 | | | | | | |
| 1_22_31 | 0.0015 | | | | | | |

Haplotype Association - highly significant values are shown in bold.

TABLE 2C

| | | | MRC POOL | MRC POOL | | | | |
|---|---|---|---|---|---|---|---|---|
| | | | CASES | CONTROLS | Allelic P | | Genotype | Genotype |
| No. | ID | | N = 372 | N = 372 | VALUE | | 1/1 | 1/2 |
| 1 | g57690 C > G | allele 1 | 199 (0.54) | 210 (0.56) | 0.418 | cases | 65 | 115 |
| | | allele 2 | 173 (0.46) | 162 (0.44) | | controls | 76 | 109 |
| 2 | g109183 G > A | allele 1 | | | | cases | 286 | 30 |
| | | allele 2 | | | | controls | 283 | 21 |
| 3 | g232794 A > G | allele 1 | | | | | | |
| | | allele 2 | | | | | | |
| 4 | g232959 C > T | allele 1 | 227 (0.61) | 233 (0.63) | 0.651 | | | |
| | | allele 2 | 145 (0.39) | 139 (0.37) | | | | |
| 5 | g259753 A > G | allele 1 | 198 (0.53) | 175 (0.47) | 0.092 | | | |
| | | allele 2 | 174 (0.47) | 197 (0.53) | | | | |
| 6 | g271466 G > C | allele 1 | 233 (0.63) | 204 (0.55) | 0.031 | cases | 193 | 270 |
| | | allele 2 | 139 (0.37) | 168 (0.45) | | controls | 169 | 277 |
| 7 | g287117 T > C | allele 1 | 346 (0.93) | 318 (0.85) | 0.001 | | | |
| | | allele 2 | 26 (0.07) | 54 (0.15) | | | | |
| 8 | g289573 A > G | allele 1 | 346 (0.93) | 320 (0.86) | 0.002 | cases | 468 | 67 |
| | | allele 2 | 26 (0.07) | 52 (0.14) | | controls | 448 | 86 |
| 9 | g289746 C > T | allele 1 | 270 (0.73) | 285 (0.77) | 0.206 | | | |
| | | allele 2 | 102 (0.27) | 87 (0.23) | | | | |
| 10 | g309989 G > A | allele 1 | 347 (0.93) | 649 (0.89) | 0.016 | | | |
| | | allele 2 | 25 (0.07) | 83 (0.11) | | | | |
| 11 | g312567 C > T | allele 1 | 635 (0.87) | 585 (0.80) | 0.003 | | | |
| | | allele 2 | 97 (0.13) | 147 (0.20) | | | | |
| 12 | g313352 G > A | allele 1 | 678 (0.93) | 651 (0.89) | 0.025 | | | |
| | | allele 2 | 54 (0.07) | 81 (0.11) | | | | |
| 13 | g313746 delT | allele 1 | 540 (0.74) | 606 (0.83) | 0.000 | | | |
| | | allele 2 | 192 (0.26) | 126 (0.17) | | | | |
| 14 | g316406 A > G | allele 1 | 268 (0.72) | 301 (0.81) | 0.004 | cases | 314 | 199 |
| | | allele 2 | 104 (0.28) | 71 (0.19) | | controls | 350 | 170 |
| 15 | g317381 C > T | allele 1 | | | | cases | 71 | 278 |
| | | allele 2 | | | | controls | 107 | 230 |
| 16 | g323237 C > T | allele 1 | 307 (0.83) | 284 (0.76) | 0.037 | cases | 372 | 156 |
| | | allele 2 | 65 (0.18) | 88 (0.24) | | controls | 346 | 169 |
| 17 | g323327 G > A | allele 1 | 327 (0.88) | 317 (0.85) | 0.282 | | | |
| | | allele 2 | 45 (0.12) | 55 (0.15) | | | | |
| 18 | g323354 A > G | allele 1 | 330 (0.89) | 331 (0.89) | 0.907 | | | |
| | | allele 2 | 42 (0.11) | 41 (0.11) | | | | |
| 19 | g323362 G > A | allele 1 | | | | | | |
| | | allele 2 | | | | | | |
| 20 | g323441 C > T | allele 1 | 307 (0.83) | 288 (0.77) | 0.082 | | | |
| | | allele 2 | 65 (0.17) | 84 (0.23) | | | | |
| 21 | g338785 C > T | allele 1 | 331 (0.89) | 309 (0.83) | 0.017 | | | |
| | | allele 2 | 41 (0.11) | 63 (0.17) | | | | |
| 22 | g341999 T > C | allele 1 | 325 (0.87) | 304 (0.82) | 0.041 | cases | 431 | 105 |
| | | allele 2 | 47 (0.13) | 68 (0.18) | | controls | 407 | 117 |
| 23 | g345179 C > T | allele 1 | 284 (0.76) | 267 (0.72) | 0.191 | | | |
| | | allele 2 | 88 (0.24) | 105 (0.28) | | | | |
| 24 | g345180 A > G | allele 1 | 285 (0.77) | 270 (0.73) | 0.222 | | | |
| | | allele 2 | 87 (0.23) | 102 (0.27) | | | | |
| 25 | g345518 T > G | allele 1 | 210 (0.56) | 185 (0.50) | 0.066 | cases | 116 | 57 |
| | | allele 2 | 162 (0.44) | 187 (0.50) | | controls | 104 | 67 |
| 26 | g345987 G > A | allele 1 | 353 (0.95) | 359 (0.96) | 0.396 | | | |
| | | allele 2 | 19 (0.05) | 13 (0.04) | | | | |
| 27 | g346355 C > T | allele 1 | 213 (0.57) | 210 (0.57) | 0.880 | | | |
| | | allele 2 | 159 (0.43) | 162 (0.43) | | | | |
| 28 | g346571 T > A | allele 1 | 348 (0.94) | 346 (0.93) | 0.770 | | | |
| | | allele 2 | 24 (0.06) | 26 (0.07) | | | | |
| 29 | g366613 A > G | allele 1 | 205 (0.55) | 169 (0.45) | 0.008 | | | |
| | | allele 2 | 167 (0.45) | 203 (0.55) | | | | |
| 30 | g371311 C > T | allele 1 | 274 (0.74) | 240 (0.65) | 0.007 | | | |
| | | allele 2 | 98 (0.26) | 132 (0.36) | | | | |
| 31 | g371348 G > A | allele 1 | 270 (0.73) | 244 (0.66) | 0.039 | cases | 260 | 245 |
| | | allele 2 | 102 (0.27) | 128 (0.34) | | controls | 248 | 242 |
| 32 | g371435 G > C | allele 1 | 279 (0.75) | 256 (0.69) | 0.074 | | | |
| | | allele 2 | 93 (0.25) | 116 (0.31) | | | | |

| | Genotype | Genotypic | HW | INDIVIDUAL | INDIVIDUAL | Allelic P |
|---|---|---|---|---|---|---|
| No. | 2/2 | P VALUE | P VALUE | CASES | CONTROLS | VALUE |
| 1 | 46 | 0.375 | 0.708 | 245 (0.54) | 261 (0.59) | 0.167 |
| | 37 | | 0.843 | 207 (0.46) | 183 (0.41) | |
| 2 | 1 | 0.311 | 0.822 | 602 (0.95) | 587 (0.97) | 0.165 |
| | 0 | | 0.533 | 32 (0.05) | 21 (0.03) | |
| 3 | | | | | | |
| 4 | | | | | | |
| 5 | | | | | | |
| 6 | 78 | 0.264 | 0.291 | 656 (0.61) | 615 (0.57) | 0.112 |
| | 91 | | 0.212 | 426 (0.39) | 459 (0.43) | |
| 7 | | | | | | |
| 8 | 5 | 0.193 | 0.143 | 1003 (0.93) | 982 (0.91) | 0.215 |
| | 3 | | 0.604 | 77 (0.07) | 92 (0.09) | |
| 9 | | | | | | |
| 10 | | | | | | |
| 11 | | | | | | |
| 12 | | | | | | |
| 13 | | | | | | |
| 14 | 27 | 0.030 | 0.527 | 827 (0.77) | 870 (0.81) | 0.009 |
| | 16 | | 0.392 | 253 (0.23) | 202 (0.19) | |
| 15 | 167 | 0.002 | 0.008 | 420 (0.41) | 444 (0.45) | 0.031 |
| | 151 | | 0.273 | 612 (0.59) | 532 (0.55) | |
| 16 | 14 | 0.102 | 0.622 | 900 (0.83) | 861 (0.80) | 0.048 |
| | 25 | | 0.457 | 184 (0.17) | 219 (0.20) | |
| 17 | | | | | | |
| 18 | | | | | | |
| 19 | | | | | | |
| 20 | | | | | | |
| 21 | | | | | | |
| 22 | 5 | 0.087 | 0.615 | 967 (0.89) | 931 (0.87) | 0.055 |
| | 13 | | 0.193 | 115 (0.11) | 143 (0.13) | |
| 23 | | | | | | |
| 24 | | | | | | |
| 25 | 67 | 0.464 | 0.097 | 289 (0.81) | 275 (0.77) | 0.254 |
| | | | 0.345 | 69 (0.19) | 81 (0.23) | |
| 26 | | | | | | |
| 27 | | | | | | |
| 28 | | | | | | |
| 29 | | | | | | |
| 30 | | | | | | |
| 31 | 36 | 0.319 | 0.030 | 765 (0.71) | 738 (0.68) | 0.257 |
| | 49 | | 0.357 | 317 (0.29) | 340 (0.32) | |
| 32 | | | | | | |

1-11. (canceled)

12. A method for diagnosing the existence of, or susceptibility to, ADAM12-mediated disease and in particular Late Onset Alzheimer's Disease (LOAD) comprising:
(a) obtaining a sample of nucleic acid encoding the protein ADAM12 from an individual to be tested;
(b) examining said nucleic acid, for any one or more of the following variants:
1. g57690 C>G
2. g109183 G>A
3. g232794 A>G
4. g232959 C>T
5. g259753 A>G
6. g271466 G>C
7. g287117 T>C
8. g289573 A>G
9. g289746 C>T
10. g309989 G>A
11. g312567 C>T
12. g313352 G>A
13. g313746 delT
14. g316406 A>G
15. g317381 C>T
16. g323237 C>T
17. g323327 G>A
18. g323354 A>G
19. g323362 G>A
20. g323441 C>T
21. g338785 C>T
22. g341999 T>C
23. g345179 C>T
24. g345180 A>G
25. g345518 T>G
26. g345987 G>A
27. g346355 C>T
28. g346571 T>A
29. g366613 A>G
30. g371311 C>T
31. g371348 G>A
32. g371435 G>C;
or a polymorphism in linkage disequilibrium therewith as described in Table 2A;
or a haplotype thereof as described in Table 2B; and
(c) where one or more of said variants and/or polymorphisms and/or haplotypes exist determining that the individual is likely to be suffering from, or susceptible to, an ADAM12-mediated disease and in particular Late Onset Alzheimer's Disease.

13. A method according to claim 12 which additionally or alternatively involves examining a polypeptide or protein encoded by said nucleic acid sample for any of the following protein sequence variants.
i) R48G; or
ii) R71Q

14. A method according to claim 12 wherein the nucleic acid sample is cDNA and the diagnostic method involves identifying any one or more of variants 1,2,16,17,21 or 26.

15. A method according to claim 13 wherein the nucleic acid sample is cDNA and the diagnostic method involves identifying any one or more of variants 1,2,16,17,21 or 26.

16. A method according to claim 12 wherein one or more of the primers listed in Table 1C are used.

17. An isolated nucleic acid molecule that encodes ADAM12 protein, or a functional part thereof, and which further comprises any one more of the following variants and/or a polymorphism in linkage disequilibrium therewith as shown in Table 2A:
1. g57690 C>G
2. g109183 G>A
3. g232794 A>G
4. g232959 C>T
5. g259753 A>G
6. g271466 G>C
7. g287117 T>C
8. g289573 A>G
9. g289746 C>T
10. g309989 G>A
11. g312567 C>T
12. g313352 G>A
13. g313746 delT
14. g316406 A>G
15. g317381 C>T
16. g323237 C>T
17. g323327 G>A
18. g323354 A>G
19. g323362 G>A
20. g323441 C>T
21. g338785 C>T
22. g341999 T>C
23. g345179 C>T
24. g345180 A>G
25. g345518 T>G
26. g345987 G>A
27. g346355 C>T
28. g346571 T>A
29. g366613 A>G
30. g371311 C>T
31. g371348 G>A
32. g371435 G>C

18. An isolated nucleic acid molecule that encodes the ADAM12 protein, or a functional part thereof, and which further comprises any one or more of the following Alzheimer's disease associated variants:
5. g259753 A>G
6. g271466 G>C
7. g287117 T>C
8. g289573 A>G
10. g309989 G>A
11. g312567 C>T
12. g313352 G>A
13. g313746 delT
14. g316406 A>G
15. g317381 C>T
16. g323237 C>T
20. g323441 C>T
21. g338785 C>T
22. g341999 T>C
25. g345518 T>G
29. g366613 A>G
30. g371311 C>T
31. g371348 G>A
32. g371435 G>C

19. An oligonucleotide for identifying a variant in the ADAM12 gene comprising an oligonucleotide listed in Table 1C.

20. An oligonucleotide for identifying a variant in the ADAM12 gene comprising an oligonucleotide that is complementary to a sequence of nucleotides either 5′ or 3′ to the variants shown in Table 1A and so is complementary to any one or more of the oligonucleotides shown in Table 1A.

21. A kit for diagnosing the existence of, or susceptibility to, ADAM12-mediated disease and in particular Late Onset Alzheimer's Disease (LOAD) comprising:
(a) at least one oligonucleotide selected from those listed in Table 1C; and/or
(b) at least one oligonucleotide complementary to any one or more of the oligonucleotides shown in Table 1A; and
optionally, one or more reagents suitable for carrying out PCR for amplifying
desired regions of a sample of DNA from an individual to be tested.

22. A vector suitable for transforming or transfecting a prokaryotic or eukaryotic cell wherein said vector comprises at least one nucleic acid molecule according to claim 17.

23. A vector suitable for transforming or transfecting a prokaryotic or eukaryotic cell wherein said vector comprises at least one nucleic acid molecule according to claim 18.

24. A prokaryotic or eukaryotic cell, or cell line, transformed or transfected with a vector according to claim 22.

25. A prokaryotic or eukaryotic cell, or cell line, transformed or transfected with a vector according to claim 23.
